(19) United States
(12) Patent Application Publication
(10) Pub. No.: US 2010/0015058 A1
(43) Pub. Date: Jan. 21, 2010

(54) RADIOLABELED BBN-RGD HETERODIMERS FOR CANCER TARGETING

(75) Inventors: ZIBO LI, LOGAN, UT (US); XIAOYUAN CHEN, UNION CITY, CA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: STANFORD UNIVERSITY, PALO ALTO, CA (US)

(21) Appl. No.: 12/491,905

(22) Filed: Jun. 25, 2009

Related U.S. Application Data
(60) Provisional application No. 61/075,359, filed on Jun. 25, 2008.

(51) Int. Cl. A61K 49/04; C07K 5/00; C07K 5/12; C07K 7/06; C07K 7/08; G01N 33/53; A61K 49/00; A61P 35/00

(52) U.S. Cl. 424/9.44; 530/317; 530/330; 530/327; 530/328; 530/329; 435/7.21; 424/9.1

(57) ABSTRACT

The present disclosure encompasses heterodimeric compositions for delivering radiolabeled and other ligands to a cell or tissue, and particularly to compositions and methods of use thereof for targeting and imaging cells and tissues expressing both an integrin and gastrin-releasing peptide receptor, in particular prostate cancer cells. The disclosure, therefore, firstly encompasses compositions that can comprise a heterodimeric probe comprising a first peptide domain comprising a moiety capable of selectively binding to an integrin; a second peptide domain comprising a moiety capable of selectively binding to a gastrin-releasing peptide receptor; a linker connecting the first peptide domain and the second peptide domain; and a prosthetic group. The first peptide domain comprises at least one tripeptide comprising the amino acid sequence of arginine-glycine-aspartate, and the second domain can be the peptide bombesin(7-14). The prosthetic group can be the fluoride isotope ¹⁸F so that the heterodimeric probe may be detected by positron emission tomography or by single photon emission computed tomography, or a metal radionuclide. The radionuclide may be attached to the probe via a chelating tether.

### CROSS-REFERENCE TO RELATED APPLICATIONS

This application claims priority to U.S. Provisional Patent Application Ser. No. 61/075,359, entitled “RADIOLABELED BBN-RGD HETERODIMERS FOR CANCER TARGETING” filed on Jun. 25, 2008, the entirety of which is hereby incorporated by reference.

### STATEMENT REGARDING FEDERALLY SPONSORED RESEARCH OR DEVELOPMENT

This disclosure was made with government support under National Cancer Institute Grant Nos: R01 CA119053, R21 CA121842, R21 CA102123, P50 CA114747, U54 CA119367, AND R24 CA93862, awarded by the U.S. National Institutes of Health of the United States government, and grant nos: W81XWH-07-1-0374, W81XWH-04-1-0697, W81XWH-06-1-0665, W81XWH-06-1-0042, AND DAMD17-03-1-0143, awarded by the U.S. Department of Defense. The government has certain rights in the disclosure.

### FIELD OF THE DISCLOSURE

The present disclosure relates to heterodimeric compositions for delivery of radiolabeled and other ligands to a cell or tissue. The disclosure further relates to methods of ligand delivery to, and imaging of, cells and tissues expressing an integrin and gastrin-releasing peptide receptor, in particular prostate cancer cells.

### SEQUENCE LISTING

The present disclosure includes a sequence listing incorporated herein by reference in its entirety.

### BACKGROUND

Prostate cancer remains one of the leading causes of cancer-related deaths in the United States and Europe (di Sant'Agnese P. A., Urology. (1998) 51: 121-124). As population life expectancy increases, so will the incidence of this disease, creating what will become an epidemic male health problem.

Over-expression of gastrin-releasing peptide receptor (GRPR) has been discovered in androgen-independent human prostate tissues, (di Sant'Agnese P. A., Urology. (1998) 51: 121-124; Chung et al., Surgery (1992) 112: 1059-1065; Glover et al., Mol. Carcinog. (2003); 37: 5-15; Vashchenko & Abrahamsson Eur. Urol. (2005) 47: 147-155), breast cancer, gastric cancer, etc. Various approaches have been explored for the imaging of in vivo GRPR expression. Bombesin (BBN), which was originally isolated from the skin of a frog, is an analog of the gastrin-releasing peptide (GRP). The truncated peptide BBN(7-14) was considered to be sufficient for the specific binding interaction with GRPR, and also is sufficiently metabolically stable for in vivo application. Several BBN peptides have been labeled with various radioisotopes for diagnosis and treatment of GRPR-positive prostate lesions (Zhang et al., J. Nucl. Med. (2006) 47: 492-501; Varvarigou et al., (2004) 19: 219-229; Zhang et al., Cancer Res. (2004) 64: 6707-6715; Smith et al., Anticancer Res. (2003) 23: 63-70; Rogers et al., Bioconjug. Chem. (2003) 14: 756-763).

¹⁸F-Labeled BBN peptides were successfully used for detecting GRPR-positive prostate cancer in vivo (Zhang et al., J. Nucl. Med. (2006) 47: 492-501). However, ¹⁸F-labeled tracers derived from monomeric BBN had a relatively low tumor accumulation and retention as well as unfavorable hepatobiliary excretion (Zhang et al., J. Nucl. Med. (2006) 47: 492-501). Therefore, modifications are desirable to obtain a better tumor-targeting effect and imaging quality.

Most solid tumors are angiogenesis-dependent and integrins are key players. In particular, integrin αᵥβ₃ was found to be necessary for the formation, survival, and maturation of new blood vessels (Friedlander et al., Science. (1995) 270: 1500-1502; Horton M. A., Int. J. Biochem. Cell Biol. (1997) 29: 721-725; Bello et al., Neurosurgery (2001) 49: 380-389). Synthetic peptides containing the arginine-glycine-aspartate (RGD) sequence motif are active modulators of cell adhesion and can bind specifically to integrin αᵥβ₃ Excellent tumor integrin-targeting efficacy and favorable in vivo kinetics were obtained for radiolabeled multimeric RGD peptides due to the polyvalency effect (Liu S., Mol. Pharm. (2006) 3: 472-487; Jung et al., J. Nucl. Med. (2006) 47: 2000-2007; Zhang et al., J. Nucl. Med. (2006) 47: 113-121; Dijkgraaf et al., Eur. J. Nucl. Med. Mol. Imaging. (2007) 34: 267-273; Dijkgraaf et al., Nucl. Med. Biol. (2007) 34: 29-35; Tucker G. C., Curr. Opin. Investig. Drugs. (2003) 4: 722-731; Li et al., J. Nucl. Med. (2007) 48:1162-1171). However, RGD peptide-based probes, including multimeric RGD peptides with high affinity for integrin αᵥβ₃, had only moderate uptake in prostate cancer models, presumably because of the insufficient expression of this receptor in prostate cancer tumors.

### SUMMARY

The present disclosure encompasses heterodimeric compositions for delivering radiolabeled and other ligands to a cell or tissue, and particularly provides compositions and methods of their use for targeting and imaging cells and tissues expressing both an integrin and gastrin-releasing peptide receptor, in particular prostate cancer cells. One aspect of the disclosure, therefore, encompasses compositions that comprise a heterodimeric probe comprising a first peptide domain comprising a moiety capable of selectively binding to an integrin; a second peptide domain comprising a moiety capable of selectively binding to a gastrin-releasing peptide receptor; a linker connecting the first peptide domain and the second peptide domain; and a prosthetic group. The prosthetic group is usefully a detectable label such as radionuclide, and optionally a therapeutically advantageous moiety.

In the embodiments of this aspect of the disclosure, the first peptide domain comprises at least one peptide comprising the amino acid sequence of arginine-glycine-aspartate, such as, for example, but not limited to, cyclo(arginine-glycine-aspartate-D-tyrosine-lysine). The moiety capable of selectively binding to a gastrin-releasing peptide receptor can comprise a fragment of the polypeptide bombesin, the fragment specifically binding to a gastrin-releasing peptide receptor. In one embodiment, the second domain is the peptide bombesin(7-14).

In some embodiments of the disclosure, the linker connecting the first peptide domain and the second peptide domain can be a glutamate residue, or an aspartate residue, and can also comprise a tether between the linker and a prosthetic group.

In the compositions of the disclosure, the prosthetic group can be a detectable label, a therapeutic agent, or a combination thereof, but in particular the prosthetic group can be a detectable label such as, but not limited to, the isotopic labels ¹⁸F, ⁶⁸Ga, ⁶⁴Cu, ⁷⁶Br, ⁸⁶Y, ¹²⁴I, ⁸⁹Zr, ¹¹¹In, ^{99m}Tc, ^{123/131}I, a fluorescent dye, a quantum dot, an alpha emitter, a beta emitter, and a gamma emitter. In especially useful embodiments, the prosthetic group comprises the fluoride isotope ¹⁸F.

Another aspect of the present disclosure provides for methods of identifying a cell or a population of cells expressing an integrin and/or a gastrin-releasing peptide receptor, comprising: contacting a cell or population of cells with an embodiment of a composition according to present disclosure, where the compositions comprise a heterodimeric probe capable of selectively binding to an integrin and to a gastrin-releasing peptide receptor of a cell; allowing the heterodimeric probe to selectively bind to at least one of an integrin and of a gastrin-releasing peptide receptor of a cell or a population of cells; and detecting the presence of the heterodimeric probe on the cell or population of cells, whereby the presence of the heterodimeric probe on the cell or population of cells indicates that the cell or population of cells has an integrin, a gastrin-releasing peptide receptor, or both an integrin and a gastrin-releasing peptide receptor thereon.

In this aspect of the disclosure, the heterodimeric probe may be, but is not limited to, being detected by positron emission tomography, by single photon emission computed tomography, fluorescent imaging, and the like, depending upon the prosthetic group attached to the heterodimeric probe.

Yet another aspect of the disclosure provides methods of delivering an agent to a cell, comprising contacting a cell or population of mammalian cells with a heterodimeric probe according to present disclosure, where the heterodimeric probe is capable of simultaneously binding to an integrin and to a gastrin-releasing peptide receptor, and where the probe further comprises an agent to be delivered to a target cell or tissue of a mammalian subject; and allowing the heterodimeric probe to bind to an integrin, a gastrin-releasing peptide receptor, or both an integrin and a gastrin-releasing peptide receptor, on the cell or population of mammalian cells, thereby delivering the agent to the cell or cells.

### BRIEF DESCRIPTION OF THE DRAWINGS

Many aspects of the disclosure can be better understood with reference to the following drawings.

FIG. 1A illustrates the strategy for enhancing the effective binding of a heterodimeric probe. Dissociation of ¹⁸F-FB-BBN-RGD from GRPR may lead to rapid recomplexation of the same ligand with integrin αᵥβ₃ (arrows).

FIG. 1B illustrates the chemical structure of ¹⁸F-FB-BBN-RGD.

FIG. 2A is a graph illustrating the inhibition of ¹²⁵I-[Tyr4]-BBN (GRPR-specific) binding to GRPR on PC-3 cells by BBN, BBN-RGD, and FB-BBN-RGD (n=3, mean±SD).

FIG. 2B is a graph illustrating the inhibition of ¹²⁵I-echistatin binding to integrin αᵥβ₃ on U87MG cells by BBN, BBN-RGD and FB-BBN-RGD (n=3, mean±SD).

FIG. 2C is a graph illustrating the cell uptake assay of ¹⁸F-FB-BBN-RGD, ¹⁸F-FB-BBN, and ¹⁸F-FB-RGD on PC-3 tumor cells (n=3, mean±SD).

FIG. 2D is a graph illustrating the cell efflux assay of ¹⁸F-FB-BBN-RGD, ¹⁸F-FB-BBN, and ¹⁸F-FB-RGD on PC-3 tumor cells (n=3, mean±SD).

FIG. 3 shows a series of digital images illustrating decay-corrected whole-body coronal small animal PET scans of an athymic male nude mouse bearing a PC-3 tumor at 30, 60, and 120 min after injection of ¹⁸F-FB-BBN-RGD, ¹⁸F-FB-BBN, or ¹⁸F-FB-RGD (3.7 MBq [100 μCi]). Images shown are 3-min static scans of a single representative animal of the 3 mice tested in each group. Tumors are indicated by arrowheads.

FIGS. 4A-4D show a series of graphs illustrating comparisons between the uptake of ¹⁸F-FB-BBN-RGD, ¹⁸F-FB-BBN, and ¹⁸F-FB-RGD in PC-3 tumor (FIG. 4A), kidneys (FIG. 4B), liver (FIG. 4C), and muscle (FIG. 4D). Dotted line in A represents the addition of ¹⁸F-FB-BBN and ¹⁸F-FB-RGD.

FIG. 4E is a graph comparing the tumor (T), muscle, kidney, and liver ratios of ¹⁸F-FB-BBN-RGD, ¹⁸F-FB-BBN, and ¹⁸F-FB-RGD at 1 hr after injection (p.i.) for athymic male nude mice bearing PC-3 tumor (n=3, mean±SD).

FIG. 5A shows a series of digital images of decay-corrected whole-body coronal small-animal PET scans of a PC-3 tumor-bearing mouse at 1 h after injection of ¹⁸F-FB-BBN-RGD and a blocking dose of c(RGDyK) (10 mg/kg of mouse body weight), BBN peptide (15 mg/kg mouse body weight), or RGD+BBN peptides (10 mg/kg for RGD and 15 mg/kg for BBN). Images shown are 3-min static scans of a single representative animal of the 3 mice tested in each group. Tumors are indicated by arrowheads.

FIG. 5B is a graph illustrating a comparison between the uptake of ¹⁸F-FB-BBN-RGD in a PC-3 tumor with or without pre-injection of blocking dose of peptides (c(RGDyK) (10 mg/kg of mouse body weight), BBN peptide (15 mg/kg mouse body weight), or RGD+BBN peptides (10 mg/kg for RGD and 15 mg/kg for BBN)). Regions of interest (ROIs) are shown as % ID/g±SD (n=3).

FIG. 6 shows a series of HPLC elution profiles illustrating the metabolic stability of ¹⁸F-FB-BBN-RGD in mouse blood and urine samples, and in liver, kidney, and PC-3 tumor homogenates at 1 hr after injection. The HPLC profile of pure ¹⁸F-FB-BBN-RGD (standard) is also shown (bottom, right).

FIGS. 7A and 7B illustrate embodiments of the heterodimeric compositions of the disclosure having the formula I (FIG. 7A) or formula II (FIG. 7B).

FIG. 8 illustrates embodiments of GRPR binding domains.

FIG. 9 illustrates a scheme for the synthesis of PEGylated BBN-RGD with a defined structure through a glutamate linker.

FIG. 10 illustrates the embodiment of the heterodimeric compositions of the disclosure having the formula III.

FIG. 11 illustrates the embodiment of the heterodimeric compositions of the disclosure having the formula IV.

FIG. 12 shows a digital image of a decay-corrected whole-body coronal small-animal PET scan of a PC-3 tumor-bearing mouse at 1 h after injection of ¹⁸F-FB-AEADP-RGD-BBN (IV).

FIG. 13 illustrates the chemical structure of the NOTA-RGD-BBN heterodimer VII.

FIG. 14A is a graph showing the inhibition of ¹²⁵I-c(RGDyK) binding to integrin αᵥβ₃ on U87MG cells by c(RGDyK) (RGD), RGD-BBN, NOTA-RGD-BBN and Aca-BBN(7-14) (BBN) (n=3, mean±SD).

FIG. 14B is a graph showing the inhibition of ¹²⁵I-[Tyr4]-BBN (GRPR-specific) binding to GRPR on PC-3 cells by Aca-BBN(7-14) (BBN), RGD-BBN, NOTA-RGD-BBN, and c(RGDyK) (RGD) (n=3, mean±SD).

FIG. 14C is a graph showing cell uptake assay of 68Ga-NOTA-RGD-BBN, 68Ga-NOTA-RGD, and 68Ga-NOTA-BBN in PC-3 tumor cells (n=3, mean±SD).

FIG. 14D is a graph showing the results of a cell efflux assay of 68Ga-NOTA-RGD-BBN, 68Ga-NOTA-RGD, and 68Ga-NOTA-BBN in PC-3 tumor cells (n=3, mean±SD).

FIG. 15 is a series of digital decay-corrected whole-body coronal small-animal PET images of PC-3 tumor-bearing mice at 30, 60 and 120 min after injecting 3.7 MBq (100 μCi) of ⁶⁸Ga-NOTA-RGD-BBN, ⁶⁸Ga-NOTA-BBN, or ⁶⁸Ga-NOTA-RGD. The images shown are 5-min static scans of a single mouse, which is representative of the four mice tested in each group (arrows PC-3 tumor).

FIGS. 16A-16C are a series of graphs showing a comparison between the quantified uptake of ⁶⁸Ga-NOTA-RGD-BBN, ⁶⁸Ga-NOTA-BBN, and ⁶⁸Ga-NOTA-RGD in a PC-3 tumor (FIG. 16A), liver (FIG. 16B), and kidneys (FIG. 16C) (n=4 per group, means±SD).

FIG. 16D is a graph showing a comparison of tumor to kidney, liver and muscle ratios of ⁶⁸Ga-NOTA-RGD-BBN, ⁶⁸Ga-NOTA-BBN, and ⁶⁸Ga-NOTA-RGD at 60 min after injection of 3.7 MBq (100 μCi) tracer in PC-3 tumor-bearing mice (n=4 per group, means±SD).

FIG. 17A is a series of digital decay-corrected whole body coronal small-animal PET images of PC-3 tumor-bearing mice at 1 h after injection of 3.7 MBq (100 μCi) of ⁶⁸Ga-NOTA-RGD-BBN and a blocking dose of c(RGDyK) (10 mg/kg), BBN (15 mg/kg), or RGD (10 mg/kg) and BBN (15 mg/kg) (n=3 or 4 per group).

FIG. 17B is a graph comparing the quantified uptake of ⁶⁸Ga-NOTA-RGD-BBN in PC-3 tumor with or without pre-injection of a blocking dose of peptide (RGD, BBN, or RGD+BBN). Data are expressed as means % ID/g±SD (n=3 or 4 per group).

FIG. 18A shows a series of digital decay-corrected whole-body coronal small-animal PET images of MDA-MB-435 tumor-bearing mice at 60 min after injection of 3.7 MBq (100 μCi) of ⁶⁸Ga-NOTA-RGD-BBN, ⁶⁸Ga-NOTA-BBN, or ⁶⁸Ga-NOTA-RGD. Images shown are 5-min static scans of a single mouse, which is representative of the four mice tested in each group (arrows indicate the MDA-MB-435 tumor).

FIG. 18B is a graph comparing the quantified uptake of ⁶⁸Ga-NOTA-RGD-BBN, ⁶⁸Ga-NOTA-RGD, and ⁶⁸Ga-NOTA-BBN in MDA-MB-435 tumor-bearing mice. Data are expressed as means % ID/g±SD (n=4 per group).

FIG. 19A shows a series of digital images of Dynamic small-animal PET imaging. The decay-corrected whole body coronal small-animal PET images are of a PC-3 tumor-bearing mouse from a 30-min dynamic scan and two static scans at 1 h and 2 h after injection of 3.7 MBq (100 μCi) of ⁶⁸Ga-NOTA-RGD-BBN (arrows indicate the PC-3 tumor).

FIG. 19B is a graph showing quantified time-% ID/g curves of a tumor and major organs after injection of 3.7 MBq (100 μCi) of 68Ga-NOTA-RGD-BBN in a PC-3 tumor-bearing mouse.

FIG. 20 is a graph showing the biodistribution of ⁶⁸Ga-NOTA-RGD-BBN (0.74 MBq per mouse) in PC-3 tumor-bearing nude mice at 0.5 h and 1 h after injection. Data are expressed as means % ID/g±SD (n=4 per group)

FIG. 21 is a series of digital images of: (a): results of immunofluorescent staining of GRPR, human integrin αᵥβᵥ, and murine integrin 3 in PC-3 tumor tissue; and (b) overlaid staining of murine CD31 and murine integrin β₃ in PC-3 tumor tissue.

FIG. 22A illustrates the chemical structure of DOTA-RGD-BBN (VIII).

FIG. 22B illustrates the chemical structure of DOTA-RGD-BBN having a metal ion chelated thereto (VIIIa).

FIG. 23A is a graph illustrating a cell uptake assay of ⁶⁴Cu-NOTA-RGD, ⁶⁴Cu-NOTA-BBN, ⁶⁴Cu-NOTA-RGD-BBN and ⁶⁴Cu-DOTA-RGD-BBN on PC-3 tumor cells (n=3, mean±SD).

FIG. 23B is a graph illustrating a cell efflux assay of ⁶⁴Cu-NOTA-RGD, ⁶⁴Cu-NOTA-BBN and ⁶⁴Cu-NOTA-RGD-BBN on PC-3 tumor cells (n=3, mean±SD).

FIG. 24 is a series of digital decay-corrected whole-body coronal small-animal PET images of PC-3 tumor-bearing mice at 30 min, 1 h, 4 h and 20 h after injection of ˜5.5 MBq (150 μCi) of ⁶⁴Cu-NOTA-RGD, ⁶⁴Cu-NOTA-BBN, ⁶⁴Cu-NOTA-RGD-BBN, ⁶⁴Cu-NOTA-RGD+⁶⁴Cu-NOTA-BBN, or ⁶⁴Cu-DOTA-RGD-BBN. Images shown were static scans of a single mouse representative of the 4 mice tested in each group. Arrows indicate the presence of PC-3 tumors.

FIG. 25A is a series of decay-corrected whole-body coronal small-animal PET images of 4T1 murine breast cancer-bearing mice at 2 h after injection of 3.7 MBq (100 μCi) ⁶⁴Cu-NOTA-RGD (RGD), ⁶⁴Cu-NOTA-BBN (BBN) or ⁶⁴Cu-NOTA-RGD-BBN(RGD-BBN). Images shown are 5-min static scans of a single mouse, which is representative of the 3 mice tested in each group. Arrows indicate the presence of 4T1 tumors.

FIG. 25B is a graph illustrating a comparison of the quantified tumor uptake of ⁶⁴Cu-NOTA-RGD, ⁶⁴Cu-NOTA-BBN and ⁶⁴Cu-NOTA-RGD-BBN in 4T1 tumor-bearing mice. ROIs are shown as % ID/g±SD (n=3/group).

FIG. 26A is a series of digital i decay-corrected whole-body coronal small-animal PET images of PC-3 tumor-bearing mice at 1 h after injection of about 5.5 MBq (150 μCi) ⁶⁴Cu-NOTA-RGD-BBN and a blocking dose of c(RGDyK), BBN peptide, or RGD+BBN peptides (n=3/group).

FIG. 26B is a graph illustrating a comparison between uptake of ⁶⁴Cu-NOTA-RGD-BBN in PC-3 tumor with or without pre-injection of blocking doses of c(RGDyK), BBN peptide, or RGD+BBN peptides. ROIs are shown as % ID/g±SD (n=34/group).

FIG. 27 is a graph illustrating the biodistribution and blocking studies of ⁶⁴Cu-NOTA-RGD-bombesin (370 kBq/mouse) in normal BALB/c mice at 1 h after injection of tracer with or without blocking dose of RGD, bombesin, or RGD+bombesin. Data are expressed as % ID/g 6 SD (n=4/group).

FIG. 28A is a graph illustrating the labeling conditions of NOTA-RGD-BBN and DOTA-RGD-BBN. NOTA-RGD-BBN or DOTA-RGD-BBN was labeled with ⁶⁴Cu at room temperature or 42° C. for 15 min, 30 min, 1 h and 2 h. The labeling yields were then detected by radio-HPLC.

FIG. 28B is a graph illustrating the cell uptake comparison of ⁶⁴Cu-NOTA-RGD-BBN and ⁶⁴Cu-NOTA-RGD-BBN on PC-3 tumor cells. Data is expressed as percent added dose (% AD) (means±SD, n=3).

FIG. 28C is a graph illustrating cell activity-retention of ⁶⁴Cu-NOTA-RGD-BBN, ⁶⁴Cu-NOTA-RGD and ⁶⁴Cu-NOTA-BBN in the efflux study. Data is expressed as percent added dose (% AD) (means±SD, n=3).

FIG. 29 is a series of graphs showing a comparison of tumor with blood, liver and kidney ratio of ⁶⁴Cu-NOTA-RGD, ⁶⁴Cu-NOTA-BBN, ⁶⁴Cu-NOTA-RGD-BBN, or ⁶⁴Cu-NOTA-RGD+⁶⁴Cu-NOTA-BBN at 30 min, 1 h and 20 h after injection of approximately 5.5 MBq (150 μCi) tracer in PC-3 tumor-bearing mice (n=4/group, mean±SD).

FIG. 30 shows a series of graphs comparing tumor with blood, liver and kidney ratio of ⁶⁴Cu-NOTA-RGD-BBN or ⁶⁴Cu-DOTA-RGD-BBN at 30 min and 20 h after injection of approximately 5.5 MBq (150 μCi) tracer in PC-3 tumor-bearing mice (n=4/group, mean±SD).

FIG. 31 shows digital images resulting from the immunofluorescent staining of gastrin-releasing peptide receptor (GRPR) and murine integrin β₃ for 4T1 tumor tissue.

FIG. 32 illustrates a scheme for the synthesis of Glu-RGD-BBN. The protected linear peptide was assembled on a Rink Amide MBHA resin. (A) Removal of Fmoc protecting group and attach Fmoc-Glu-OAII where the α-carboxylate group was orthogonally protected as allyl ester. (B) Deprotection of OAII with Pd(Ph₃P)₄/CHCl₃/AcOH/NMM, activation of the δ-carboxylate group on Glu with O—(N-Succinimidyl)-1,1,3,3-tetramethyluronium tetrafluoroborate (TSTU), and coupling with cyclo(-Arg-Gly-Asp-DTyr-Lys-) through the Lys side chain ε-amine group. (C) Removal of Fmoc with piperidine and detaching/deprotecting the peptide with TFA/EDT/TIS to afford the Glu-RGD-BBN.

FIG. 33 illustrates the chemical structure of ¹⁸F-FB-PEG₃-Glu-RGD-BBN. The cyclic RGD peptide c(RGDyK) was connected with Aca-BBN(7-14) through a glutamate linker with RGD attached to the α-carboxylate and BBN attached to the γ-carboxylate. Labeling with ¹⁸F was carried out via acylation of the amino group at the PEG₃ spacer using ¹⁸F-SFB as synthon.

FIGS. 34 A and 34B are graphs illustrating: (FIG. 34A) inhibition of ¹²⁵I-c(RGDyK) binding to integrin αᵥβ₃ on U87MG cells by c(RGDyK), PEG₃-Glu-RGD-BBN, and FB-PEG₃-Glu-RGD-BBN: □, c(RGDyK) (IC₅₀=11.19±1.44); ▴, PEG₃-Glu-RGD-BBN (IC₅₀=10.80±1.46); ∘, FB-PEG₃-Glu-RGD-BBN (IC₅₀=13.77±1.82). (n=3, mean±SD); (FIG. 34B) inhibition of ¹²⁵I-[Tyr⁴]-BBN (GRPR-specific) binding to GRPR on PC-3 cells by Aca-BBN (7-14), PEG₃-Glu-RGD-BBN, and FB-PEG₃-Glu-RGD-BBN: □, Aca-BBN (7-14), (IC₅₀=78.96±2.12); ▴, PEG₃-Glu-RGD-BBN (IC₅₀=85.45±1.95); ∘, FB-PEG₃-Glu-RGD-BBN (IC₅₀=73.28±1.57). (n=3, mean±SD).

FIGS. 35A and 35B are graphs showing: (FIG. 35A) cell uptake assay of ¹⁸F-PEG₃-Glu-RGD-BBN on PC-3 tumor cells at 4° C.: □, Without blocking; ▴, Blocking with c(RGDyK); x, Blocking with Aca-BBN (7-14); ∘, Blocking with c(RGDyK) and Aca-BBN (7-14). (n=3, mean±SD); (FIG. 35B) cell uptake assay of ¹⁸F-PEG₃-Glu-RGD-BBN on PC-3 tumor cells at 37° C.: ∘, Without blocking; , Blocking with Glu-RGD-BBN; ▴, Cell uptake of ¹⁸F-PEG₃-Glu-RGD-BBN at 4° C. (for comparison); Δ, The internalized fraction of ¹⁸F-PEG₃-Glu-RGD-BBN (calculated by subtracting the cell uptake at 4° C. from the uptake at 37° C. at each time point) (n=3, mean±SD).

FIG. 36A is a series of digital coronal microPET images and the radioactivity accumulation quantification (FIG. 36B) in selected organs of the PC-3 tumor-bearing mice at 30 min, 60 min and 120 min after injection of 3.7 MBq (100 μCi) of ¹⁸F-PEG₃-Glu-RGD-BBN. Arrows indicate the presence of PC-3 tumors. All microPET images were decay-corrected. FIG. 36C shows the calculated tumor/non-tumor (T/NT) ratios from FIG. 36B.

FIGS. 37A-37C show a series of digital decay-corrected whole-body coronal microPET images of PC-3 tumor-bearing mice at 1 h after injection of 3.7 MBq (100 μCi) ¹⁸F-PEG₃-Glu-RGD-BBN and a blocking dose of c(RGDyK) (10 mg/kg of mouse body weight), BBN peptide (15 mg/kg mouse body weight), or RGD+BBN peptides (10 mg/kg for RGD and 15 mg/kg for BBN) (n=3). FIGS. 37B and 37C show a comparison between uptake of ¹⁸F-PEG₃-Glu-RGD-BBN in PC-3 tumor (FIG. 37B), or blood (FIG. 37C) with or without pre-injection of blocking dose of peptides (c(RGDyK), BBN peptide, or RGD+BBN peptides). ROIs are shown as % ID/g±SD (n=3).

FIG. 38 is a graph showing the time-activity curves of major organs in a PC-3 tumor-bearing nude mouse after intravenous injection of 3.7 MBq (100 μCi) ¹⁸F-PEG₃-Glu-RGD-BBN. Data were derived from a multiple time-point microPET study.

FIG. 39 is a graph showing biodistribution studies of ¹⁸F-PEG₃-Glu-RGD-BBN in PC-3 tumor-bearing nude mice at 1 h post-injection. The uptakes of ¹⁸F-PEG₃-Glu-RGD-BBN in PC-3 tumors and major organs measured by microPET and biodistribution were compared. Data are expressed as % ID/g±SD (n=4).

FIGS. 40A and 40B are graphs showing GRPR and integrin αᵥβ₃ levels in different breast cancer cell lines as determined by cell binding assay using ¹²⁵-I-[Tyr4]BBN (FIG. 40A) or ¹²⁵I-c(RGDyK) (FIG. 40B) as the radioligand (means±SD, n=5).

FIG. 40C shows a series of digital images from immunofluorescent staining for GRPR, human integrin αᵥβ₃ and murine integrin β₃ in MDA-MB435 and T47D tumor tissues.

FIGS. 41A and 41B are graphs showing the results of cell uptake assays for ¹⁸F-FB-PEG₃-RGD-BBN, ⁶⁴Cu-NOTA-RGD-BBN and ⁶⁸Ga-NOTA-RGD-BBN on T47D (FIG. 41A) or MDA-MB435 (FIG. 41B) tumor cells (means±SD, n=3).

FIG. 42 shows a series of digital decay-corrected whole-body coronal microPET images of T47D (T) and MDA-MB-435 (M) tumor-bearing mice at 30 min, 1 h, 2 h, 4 h and 24 h after injection of 3.7˜5.5 MBq (100-150 μCi) ⁶⁴Cu-NOTA-RGD-BBN, ¹⁸F-FB-PEG₃-RGD-BBN, or ⁶⁸Ga-NOTA-RGD-BBN. Images shown are static scans of a single mouse, which was representative of the 4 mice tested in each group. Arrows indicate the presence of T47D (T) or MDA-MB-435 (M) tumors.

FIGS. 43A-43E show a series of graphs illustrating a comparison between the uptake of ¹⁸F-FB-PEG₃-RGD-BBN, ⁶⁴Cu-NOTA-RGD-BBN and ⁶⁸Ga-NOTA-RGD-BBN in T47D tumor (FIG. 43A), MDA-MB-435 tumor (FIG. 43B), blood (FIG. 43C), kidneys (FIG. 43D), and liver (FIG. 43E) after injection of 3.7˜5.5 MBq (100-150 μCi) tracer in T47D or MDA-MB-435 tumor-bearing mice (n=4-8, mean±SD).

FIG. 43F is a graph showing a comparison of tumor (T) with blood, kidney, liver and muscle ratio of ¹⁸F-FB-PEG₃-RGD-BBN, ⁶⁴Cu-NOTA-RGD-BBN and ⁶⁸Ga-NOTA-RGD-BBN at 60 min after injection of 3.7˜5.5 MBq (100-150 μCi) tracer in T47D tumor-bearing mice (n=4/group, mean±SD).

FIG. 44A shows a series of digital decay-corrected whole-body coronal microPET images of MDA-MB435 tumor-bearing mice at 30 min after injection of 3.7 MBq (100 μCi) ¹⁸F-FB-BBN⁶⁴Cu-NOTA-BBN, or ⁶⁸Ga-NOTA-BBN. Images shown are static scans of a single mouse, which is representative of the 3 mice tested in each group. Arrows indicate the presence of MDA-MB435 tumors.

FIG. 44B is a graph showing a comparison between the quantified uptake of ¹⁸F/⁶⁴Cu/68Ga labeled RGD-BBN tracers with ¹⁸F/⁶⁴Cu/68Ga labeled BBN tracers in MDA-MB-435 tumors (n=3-4/group, mean±SD).

FIG. 45A is a series of HPLC traces showing the in vitro serum stability of ⁶⁸Ga-NOTA-RGD-BBN, ⁶⁴Cu-NOTA-RGD-BBN or ¹⁸F-FB-PEG₃-RGD-BBN after incubating in fetal bovine serum (FBS) for 2 hour at room temperature.

FIG. 45B is a series of HPLC traces showing the metabolic stability of ⁶⁸Ga-NOTA-RGD-BBN, ⁶⁴Cu-NOTA-RGD-BBN or ¹⁸F-FB-PEG₃-RGD-BBN in mice urine at 1 h after injection (n=2).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### ABBREVIATIONS

BBN, bombesin; GRPR, gastrin-releasing peptide receptor; GRP, gastrin-releasing peptide; RGD, argine-glycine-aspartate; FB, fluorobenzoate; PET, positron emission tomography; SPECT, single photon emission computed tomography; GP, glycoprotein; TFA, trifluoroacetic acid; ACN, acetonitrile. NOTA, 1,4,7-triazacyclononane-1,4,7-triacetic acid; DOTA (1,4,7,10-tetraazacyclododecane-N,N′,N″,N′″-tetracetate; PEG₃, 11-amino-3,6,9-trioxaundecanoic acid; Aca-BBN(7-14), Aca-Gln-Trp-Ala-Val-Gly-His-Leu-Met-NH₂; c(RGDyK), cyclo(Arg-Gly-Asp-D-Tyr-Lys); RGD-BBN, cyclo(Arg-Gly-Asp-D-Tyr-Lys)-Glu-(Aca-Gln-Trp-Ala-Val-Gly-His-Leu-Met-NH₂); SFB, N-succinimidyl-4-fluorobenzoate.

### DEFINITIONS

In describing and claiming the disclosed subject matter, the following terminology will be used in accordance with the definitions set forth below.

The terms “agent” and “therapeutic agent’ as used herein refer to a compound which is desired to be delivered to a target cell or tissues that have the GPRP and integrin ligands to which the heterodimeric compositions of the present disclosure can selectively bind. Such agents would be useful in modulating the proliferation of cells such as cancer cells, and may be useful in destroying such cells. It is further contemplated that an agent or therapeutic agent may be included in a heterodimeric construct of the present disclosure that further comprises a radiolabeled or otherwise tagged prosthetic group for monitoring the location of the construct on a cell or in the tissues of a treated animal or human host.

The term “cell or population of cells” as used herein refers to an isolated cell or plurality of cells excised from a tissue or grown in vitro by tissue culture techniques. In the alternative, a population of cells may also be a plurality of cells in vivo in a tissue of an animal or human host.

The term “contacting a cell or population of cells” as used herein refers to delivering a composition such as, for example, a heterodimeric probe composition according to the present disclosure with or without a pharmaceutically or physiologically acceptable carrier to an isolated or cultured cell or population of cells, or administering the probe in a suitable pharmaceutically acceptable carrier to an animal or human host. Thereupon, it may be systemically delivered to the target and other tissues of the host, or delivered to a localized target area of the host. Administration may be, but is not limited to, intravenous delivery, intraperitoneal delivery, intramuscularly, subcutaneously or by any other method known in the art. One method is to deliver the composition directly into a blood vessel leading immediately into a target organ or tissue such as a prostate, thereby reducing dilution of the probe in the general circulatory system.

The term “fluorophore” as used herein refers to a component of a molecule that causes a molecule to be fluorescent. It is a functional group in a molecule which will absorb energy of a specific wavelength and re-emit energy at a different (but equally specific) wavelength. The amount and wavelength of the emitted energy depend on both the fluorophore and the chemical environment of the fluorophore. Fluorescein isothiocyanate (FITC), a reactive derivative of fluorescein, has been one of the most common fluorophores chemically attached to other, non-fluorescent, molecules to create new fluorescent molecules for a variety of applications. Other historically common fluorophores are derivatives of rhodamine (TRITC), coumarin, and cyanine. Newer generations of fluorophores such as the ALEXA FLUORS™ and the DYLIGHT FLUORS™ are generally more photostable, brighter, and less pH-sensitive than other standard dyes of comparable excitation and emission.

The term “heterodimer” as used herein refers to a molecule comprising two identifiable domains or regions having different functions, amino acid sequences, or other properties. The heterodimers of the present disclosure may comprise a first domain that includes the tri-amino acid sequence arginine-glycine-aspartate (RGD; SEQ ID NO.: 1) that is capable of selectively binding to an integrin, and a second domain comprising a fragment of the polypeptide bombesin, and which is capable of selectively binding to a gastrin-releasing peptide receptor. The first and second domains may be contiguous, or connected by a linker molecule, wherein the first domain may be linked to the amino or the carboxyl end of the bombesin fragment. An especially advantageous heterodimer of this disclosure comprises a first domain linked directly, or by a linker moiety, to the amino end of the second domain.

The term “host” as used herein refers to a mammalian or non-mammalian animal, or human, subject or patient in receipt of a composition according to the present disclosure.

The term “integrin” as used herein refers to a widely expressed family of calcium or magnesium dependent α or β heterodimeric cell surface receptors that bind to extracellular matrix adhesive proteins such as fibrinogen, fibronectin, vitronectin, and osteopontin. The integrin receptors are transmembrane glycoproteins (GP's) known for their large extracellular domains and are classified by at least 8 known β subunits and 14α subunits. For example, the β₁ subfamily has the largest number of integrins, where the various a subunits associate with various β subunits: β₃, β₅, β₆, and β₈. Some of the disease states that have a strong αᵥβ₃, αᵥβ₅, and α_{IIb}β₃ (also referred to as GPIIb/IIIa) integrin component in their etiologies are unstable angina, thromboembolic disorders or atherosclerosis (GPIIb/IIIa); thrombosis or restenosis (GPIIb/IIIa or αᵥβ₃); restenosis (dual αᵥβ₃/GPIIb/IIIa); rheumatoid arthritis, vascular disorders or osteoporosis (αᵥβ₃); tumor angiogenesis, tumor metastasis, tumor growth, multiple sclerosis, neurological disorders, asthma, vascular injury or diabetic retinopathy (αᵥβ₃ or αᵥβ₅); and, angiogenesis (dual αᵥβ₃/αᵥβ₅).

The term “linker” as used herein refers to any molecular structure that connects the two functionally dissimilar domains that together constitute the single heterodimeric construct of the present disclosure. A particularly useful linker is, for example, a glutamate moiety, the carboxyl groups of which may form peptide bonds with amine groups on each of the two domains to be joined. It is also contemplated that a prosthetic group such as, but not limited to, a fluorobenzoate may attach to a glutamate linker, for example, through the α-amino group of a glutamate linker.

It is further contemplated that a “linker’ may refer to a molecular structure that conjugates two similarly functioning domains, such as, but not limited to, the multimeric domains comprising at least one tripeptide structure comprising the amino acid sequence arginine-glycine-aspartate (SEQ ID NO.: 1). It is also contemplated that a linker molecule suitable for use in the heterodimeric compositions of the present disclosure can be, but is not limited to, a dicarboxylic acid that further includes at least one available group, such as an amine group, for conjugating to a prosthetic group. However, it is also contemplated that other functional side groups may substitute for the amine group to allow for the linking to selected prosthetic groups. Exemplary dicarboxylic acids include, but are not limited to, aspartate, glutamate, and the like, and can have the general formula (HOOC)—(CH₂)ₙ—(CHNH₂⁺)—(CH₂)ₘ—(COOH), where n and m are each independently 0, or an integer from 1 to about 10. It is further considered within the scope of the disclosure for the linker to be a multimer, or a combination, of at least two such dicarboxylic acids. For example, such linker molecules may include, but are not limited to, (aspartate)ₓ, (glutamate)_{y}, or a combination thereof, where adjacent amino acids can be joined by peptide bonds, and the like. The subscripts x and y are each independently 0, or an integer from 1 to about 12.

The term “peptide” as used herein refers to short polymers formed from the linking, in a defined order, of α-amino acids. The link between one amino acid residue and the next is known as an amide bond or a peptide bond. Proteins are polypeptide molecules (or consist of multiple polypeptide subunits). The distinction is that peptides are short and polypeptides/proteins are long. There are several different conventions to determine these. Peptide chains that are short enough to be made synthetically from the constituent amino acids are called peptides, rather than proteins, with one dividing line at about 50 amino acids in length.

Modifications and changes can be made in the structure of the peptides of this disclosure and still result in a molecule having similar characteristics as the peptide (e.g., a conservative amino acid substitution). For example, certain amino acids can be substituted for other amino acids in a sequence without appreciable loss of activity. Because it is the interactive capacity and nature of a peptide that defines that peptide's biological functional activity, certain amino acid sequence substitutions can be made in a peptide sequence and nevertheless obtain a peptide with like properties.

In making such changes, the hydropathic index of amino acids can be considered. The importance of the hydropathic amino acid index in conferring interactive biologic function on a peptide is generally understood in the art. It is known that certain amino acids can be substituted for other amino acids having a similar hydropathic index or score and still result in a peptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant peptide, which in turn defines the interaction of the peptide with other molecules, such as enzymes, substrates, receptors, antibodies, antigens, and the like. It is known in the art that an amino acid can be substituted by another amino acid having a similar hydropathic index and still obtain a functionally equivalent peptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biologically functional equivalent peptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent peptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a peptide as set forth above. In particular, embodiments of the peptides can include variants having about 50%, 60%, 70%, 80%, 90%, and 95% sequence identity to the peptide of interest.

The term “pharmaceutically acceptable carrier” as used herein refers to a diluent, adjuvant, excipient, or vehicle with which a heterodimeric probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the heterodimeric probes and pharmaceutically acceptable carriers preferably should be sterile. Water is a useful carrier when the heterodimeric probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “physiologically acceptable” as used herein refers to a composition that, in contact with a cell, isolated from a natural source or in culture, or a tissue of a host, has no toxic effect on the cell or tissue.

The term “positron emission tomography” as used herein refers to a nuclear medicine imaging technique that produces a three-dimensional image or map of functional processes in the body. The system detects pairs of gamma rays emitted indirectly by a positron-emitting radioisotope, which is introduced into the body on a metabolically active molecule. Images of metabolic activity in space are then reconstructed by computer analysis. Using statistics collected from tens-of-thousands of coincidence events, a set of simultaneous equations for the total activity of each parcel of tissue can be solved by a number of techniques, and a map of radioactivities as a function of location for parcels or bits of tissue may be constructed and plotted. The resulting map shows the tissues in which the molecular probe has become concentrated. Radioisotopes used in PET scanning are typically isotopes with short half lives such as carbon-11 (about 20 min), nitrogen-13 (about 10 min), oxygen-15 (about 2 min), and fluorine-18 (about 10 min). PET technology can be used to trace the biologic pathway of any compound in living humans (and many other species as well), provided it can be radiolabeled with a PET isotope. The half life of fluorine-18 is long enough such that fluorine-18 labeled radiotracers can be manufactured commercially at an offsite location.

The term “prosthetic group’ as used herein refers to a chemical moiety conjugated to a region of the heterodimeric constructs of the present disclosure. The prosthetic group may include a “tether” and a detectable moiety such as, but not limited to a radiolabel, a fluorescent dye, and the like. In addition, or in place of, the detectable moiety, the prosthetic group may be an agent such as a therapeutic agent required to be targeted to a cell bearing the GPRP and integrin ligands to which the heterodimeric compositions of the present disclosure can selectively bind.

The term “radiolabel prosthetic group” as used herein refers to a moiety conjugated to a heterodimer of the present disclosure, where the moiety includes a radiolabel. Most advantageous for the heterodimers of the disclosure are moieties that may be attached to a tether such as, but not limited to, a benzoate derivative. The prosthetic group may have a radiolabel attached thereto. For example, one useful prosthetic group is fluorobenzoate, where the carboxyl group of the benzoate may be conjugated to the α-amino group of a glutamate linker, and the fluoride is the isotope ¹⁸F detectable by such as PET.

The heterodimer constructs according to the present disclosure can be labeled with a radionuclide suitable for imaging by such as, but not limited to, Positron Emission Tomography (PET) or Single Photon Emission Computed Tomography (SPECT), or for the detection of, or the therapeutic use of, alpha-(α), beta-(β), and gamma (γ)-emitting isotopes. Some exemplary embodiments of elements that can be used as labels in the present disclosure include, but are not limited to, F-19 (F-18), C-12 (C-11), 1-127 (1-125, 1-124, 1-131, 1-123), CI-36 (CI-32, CI-33, CI-34), Br-80 (Br-74, Br-75, Br-76, Br-77, Br-78), Re-185/187 (Re-186, Re-188), Y-89 (Y-90, Y-86), Lu-177, and Sm-153, as well as those described in the figures. Imaging probes for use in the probes of the present disclosure can be labeled with one or more radioisotopes, preferably including, but not limited to, ¹¹C, ¹⁸F, ⁷⁶Br, ¹²³I, ¹²⁴I, or ¹³¹I, and are suitable for use in peripheral medical facilities and PET clinics. In particular embodiments, for example, the PET isotope can include, but is not limited to, ^{64/61}Cu, ¹²⁴I, ^{76/77}Br, ⁸⁸Y, ⁸⁹Zr, and ⁶⁸Ga.

The term “target” as used herein can refer to a polypeptide for which it is desired to detect. The target polypeptide for use in the methods herein disclosed may be an isolated polypeptide, a polypeptide immobilized on a solid support or in free solution. Alternatively, the target polypeptide may be on a cell surface, the cell being isolated from an animal host, a cultured cell or a cell or population of cells in a tissue of an animal.

The term “tether” as used herein refers to a linker joining a prosthetic group such as, but not limited to, a radioactive label to a molecular structure. For example, and not intended to be limiting, a tether moiety can be an N-succinimidyl benzoate that can conjugate to an amine side group of another moiety such as a linker connecting the two heteropeptide domains. In one embodiment, a tether such as a benzoate may be substituted at the 4-position, for example, with the radiolabel 18-fluoride prosthetic group.

### Discussion

Molecular imaging of cancer is a fast growing research field. Molecular imaging technologies have demonstrated great benefits for better understanding cancer biology, as well as for facilitating cancer drug development and cancer early detection. Development of novel imaging methods and molecularly targeted probes will allow not only to locate a tumor, but also to visualize the expression and activity of specific molecular targets and biological processes in a tumor.

In recent years, it has been learned that some cancer cells contain gastrin releasing peptide (GRP) receptors (GRP-R) of which there are a number of subtypes. In particular, it has been shown that several types of cancer cells have over-expressed or uniquely expressed GRP receptors. GRP and GRP analogues can selectively bind to the GRP receptor family. One GRP analogue is bombesin (BBN), (i.e., tetradecapeptide) isolated from frog skin that can bind to GRP receptors with high specificity and with an affinity similar to GRP.

GRP receptors have been shown to be over-expressed or uniquely expressed on several types of cancer cells. In addition to being seen in prostate cancers, GRPR is also expressed in almost 60% of primary breast carcinoma cases and in almost all infiltrated lymph nodes. Extremely high numbers of GRPRs have also been detected in gastrointestinal stromal tumors. Binding of GRP receptor agonists (also autocrine factors) increases the rate of cell division of these cancer cells. The fragments of bombesin useful in the embodiments of the heterodimers of the present disclosure contain either the same primary structure of the bombesin GPR binding region, i.e. bombesin(7-14) (SEQ ID NO.: 7) or bombesin(8-14) (SEQ ID NO.: 6), or similar primary structures, with specific amino acid substitutions, that will specifically bind to GRP receptors. Compounds containing this bombesin GPR binding region (or binding moiety), when covalently linked to other groups may also be referred to as bombesin conjugates.

Integrin αᵥβ₃ is expressed in GRPR-positive cancers, as well as many other cancer types. The application of BBN-RGD heterodimers according to the present disclosure for tumor targeting will thus be applicable to many cancer types that express both GRPR and integrin, GRPR only, or integrin only.

A dual GRPR-integrin αᵥβ₃-targeting approach according to the present disclosure provides improved imaging probes over those that recognize only a single receptor type. Accordingly, the heterodimeric probe construct ¹⁸F-FB-BBN-RGD comprising a BBN peptide motif for GRPR targeting, and an RGD peptide motif for integrin αᵥβ3 targeting, was synthesized and radiolabeled. The receptor-binding assay data demonstrated that BBN-RGD heterodimeric construct is similar to Aca-BBN(7-14) for GRPR binding, and is similar to c(RGDyK) for integrin αᵥβ₃ binding.

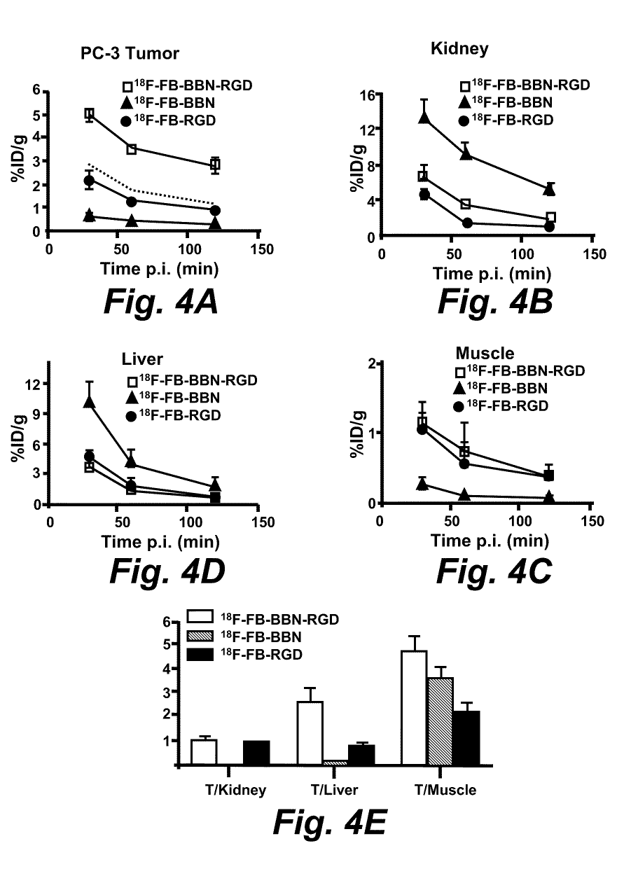

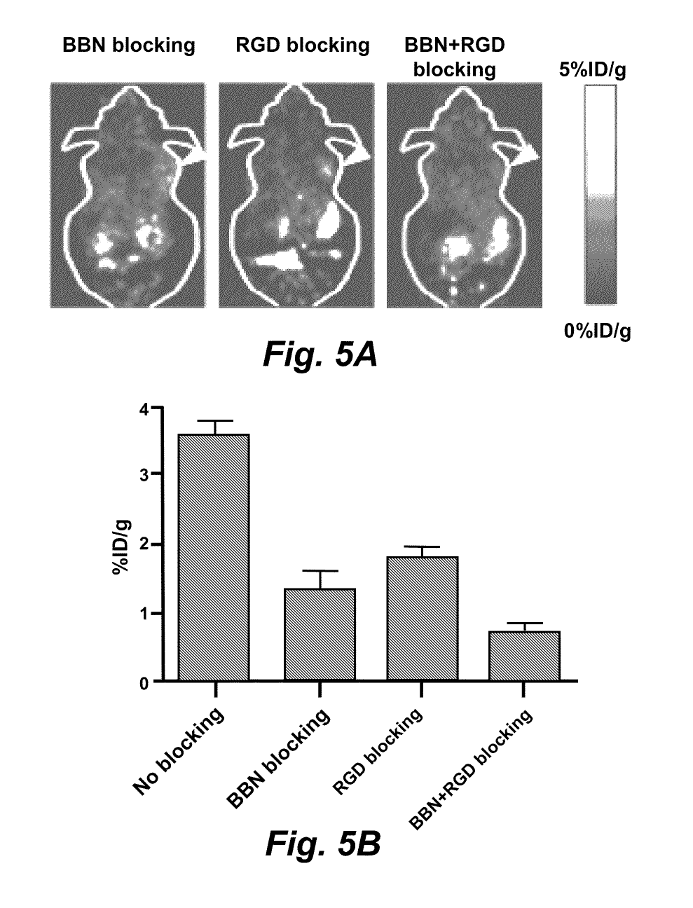

As shown in FIGS. 4A-5B, ¹⁸F-FB-RGD showed the lowest cell uptake, probably because integrin αᵥβ₃ binding does not tend to internalize the tracer. However, in vivo uptake of the ¹⁸F-FB-BBN derivative is lower even than that for the RGD derivative, which might be partially attributed to their difference in pharmacokinetics. The cell uptake of ¹⁸F-FB-BBN-RGD was slightly lower than that of ¹⁸F-FB-BBN at early time points, but was significantly higher than that of ¹⁸F-FB-RGD in PC-3 cells. ¹⁸F-FB-BBN-RGD tends to have a slower washout than ¹⁸F-FB-BBN, which might be the result of enhanced effective binding due to dual targeting.

As PC-3 tumor cells express both GRPR and integrin αᵥβ₃, the imaging quality of ¹⁸F-FB-BBN-RGD was tested in a PC-3 xenograft model. Compared with ¹⁸F-FB-BBN and ¹⁸F-FB-RGD, the PC-3 tumor uptake of ¹⁸F-FB-BBN-RGD was much higher than the sum of the monomeric tracers at all time points examined, as illustrated in FIG. 4A. The pharmacokinetics were also improved for ¹⁸F-FB-BBN-RGD compared with that of ¹⁸F-FB-BBN and ¹⁸F-FB-RGD in the liver and kidneys, which may be attributed to the differences in molecular size, charge, and hydrophilicity of these three compounds.

¹⁸F-FB-BBN-RGD also had the highest tumor to non-tumor ratios when compared with ¹⁸F-FB-BBN and ¹⁸F-FB-RGD. Overall, a synergistic effect has been observed for ¹⁸F-FB-BBN-RGD compared to probes comprising just one binding domain, and significantly higher tumor uptake and contrast have been obtained in the PC-3 tumor model. In the blocking experiment, neither non-radioactive BBN peptide nor non-radioactive RGD peptide could totally inhibit the uptake of ¹⁸F-FB-BBN-RGD in PC-3 tumor, as the tracer could still bind to the unblocked receptors. The BBN and RGD double blocking could further reduce the tumor uptake, which strongly supports the dual-receptor specificity of ¹⁸F-FB-BBN-RGD in vivo. For ¹⁸F-FB-BBN-RGD, the RGD blocking resulted in a slightly higher tumor uptake than BBN blocking, which may be due to the PC-3 cells expressing a high level of GRPR but only a medium level of integrin αᵥβ₃. Moreover, the advantage of this heterodimer tracer is apparent when only one receptor type is over-expressed. For example, the DU-145 tumor expresses a moderate level of integrin αᵥβ₃, but expresses a low level of GRPR (Cooper et al., Neoplasia (2002) 4: 191-194; Markwalder & Reubi, Cancer Res. (1999) 59: 1152-1159; Haywood-Reid et al., Prostate (1997) 31: 1-8). ¹⁸F-FB-BBN that binds to GRPR but not to integrin αᵥβ₃ is unable to provide enough tumor uptake and tumor-to-background contrast. ¹⁸F-FB-BBN-RGD, on the other hand, had a tumor uptake similar to that of ¹⁸F-FB-RGD, but had a significantly lower background.

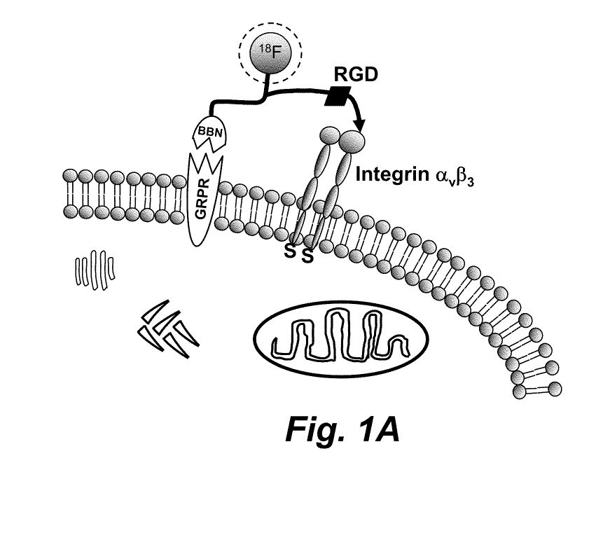

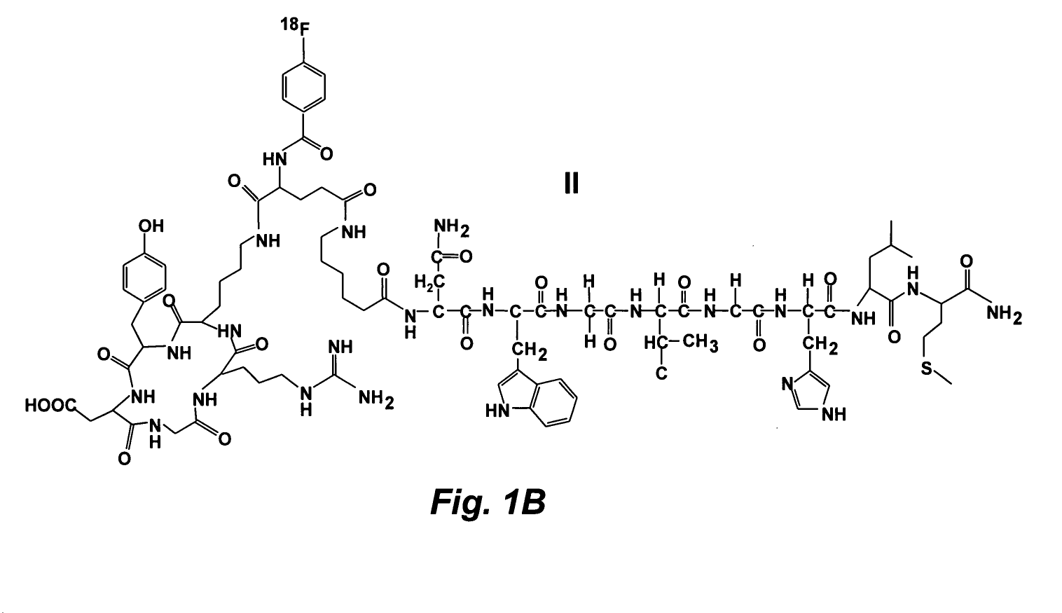

In the metabolic stability study, the metabolites of ¹⁸F-FB-BBN-RGD may be determined primarily by the FB unit and BBN sequence as the cyclic RGD-containing pentapeptide is highly stable in vivo. The structure of the BBN-RGD heterodimer, as illustrated in FIG. 1B, indicates that the peptide likely binds to either GRPR or integrin αᵥβ₃, since a linker comprising just a single glutamate moiety is too short to allow simultaneous GRPR and integrin αᵥβ₃ binding. Thus, the total number of potential binding sites for this ligand is the sum of GRPR and integrin αᵥβ₃, higher than that for RGD peptide and for BBN analogs alone. The advantage of BBN-RGD over the individual BBN or RGD peptides is not only the increased number of receptors for signal amplification, but also the binding kinetics contribute to the in vivo behavior of ¹⁸F-FB-BBN-RGD. Assuming that the ligand binds to GRPR through the BBN moiety, the remaining RGD moiety will then be in close vicinity of integrin αᵥβ₃. The dissociation of BBN-RGD from GRPR can then lead to rapid recomplexation of the same ligand, but this time with integrin αᵥβ₃. The effect is that the binding of the heterodimeric probe to the GPRP/integrin-containing cells is synergistically greater than if the probe had only one binding domain, either the RGD peptide or a BBN fragment.

If, however, the heterodimer is initially bound to integrin instead, the dissociation of the RGD motif from integrin will reorient the BBN-RGD to bind to GRPR, resulting in an apparent low off-rate of the ligand binding. Both the increased number of binding sites and the apparent low off-rate of the dual-receptor-targeting ligand may be expected to have enhanced tumor uptake and retention as compared with those single-receptor-recognizing ligands. The added molecular size and change of overall molecular charge and hydrophilicity can also have effects on the in vivo kinetics of the resulting probes.

For integrin binding, multimeric RGD peptides can be advantageous over monomeric counterparts in terms of receptor-binding affinity in vitro and tumor-targeting efficacy in vivo, most likely due to the so-called “multivalency effect” (Liu S. Mol. Pharm. 2006; 3:472-487; Li et al., J. Nucl. Med. 2007; 48:1162-1171; Wu et al., J. Nucl. Med. 2007; 48:1536-1544). It is contemplated, therefore, that in the various embodiments of the present disclosure, BBN analogs may be linked with dimeric or oligomeric RGD tripeptide units through a linker molecule such as, but not limited to, a glutamate linker.

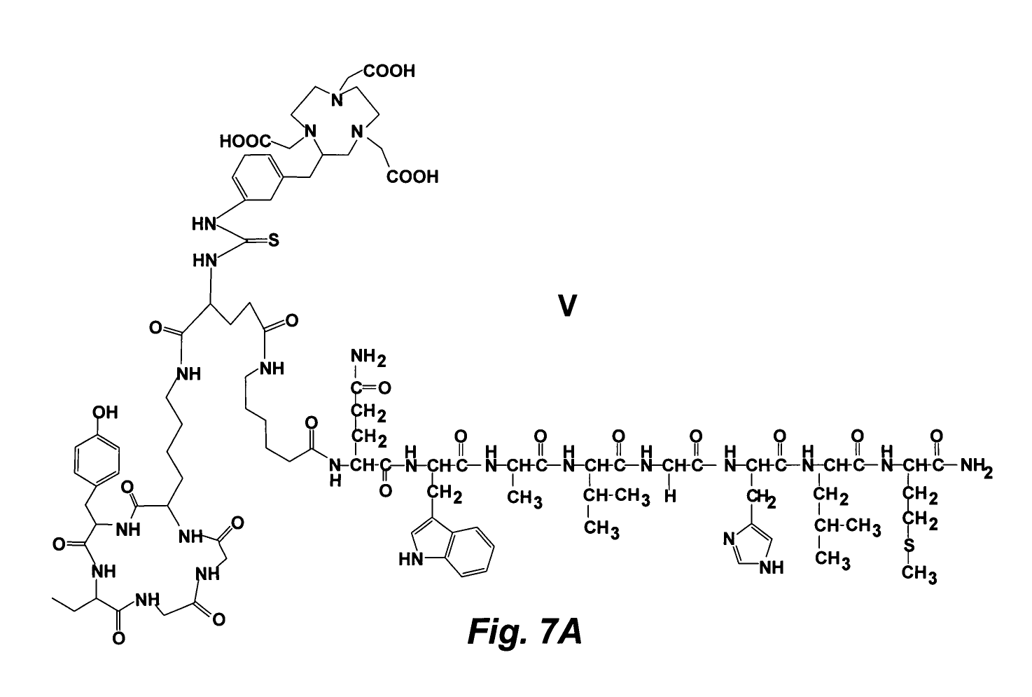

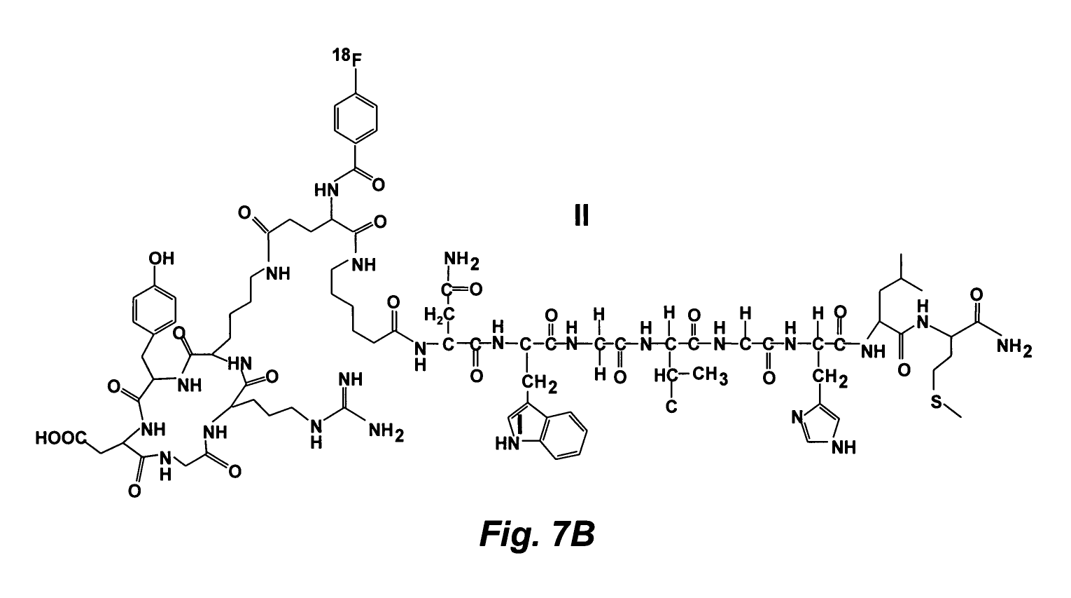

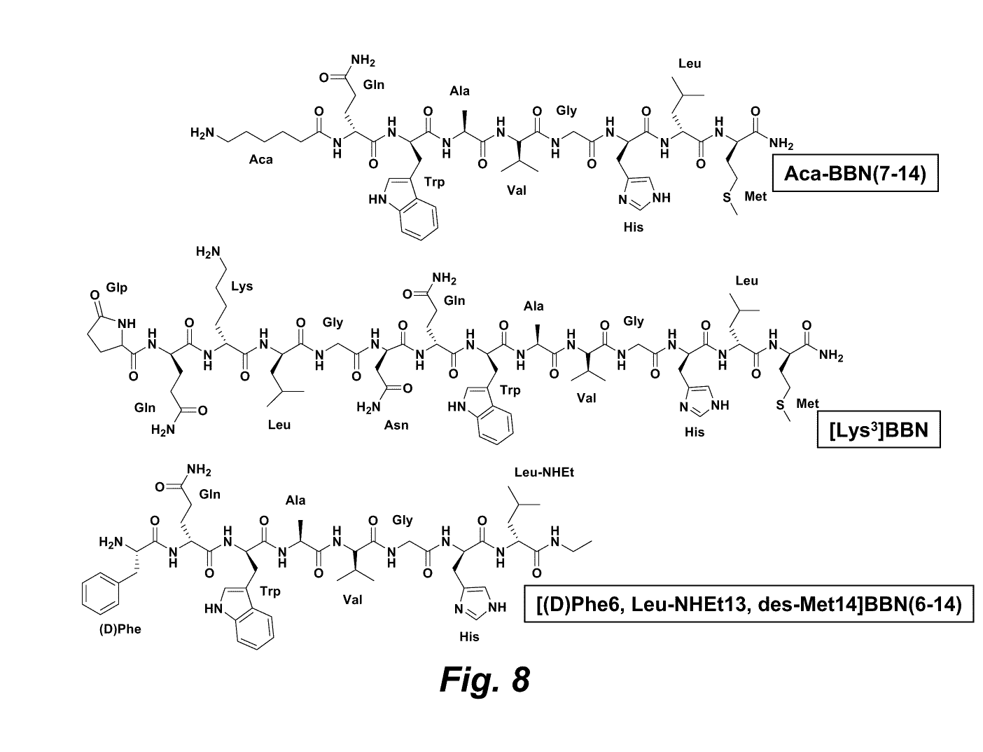

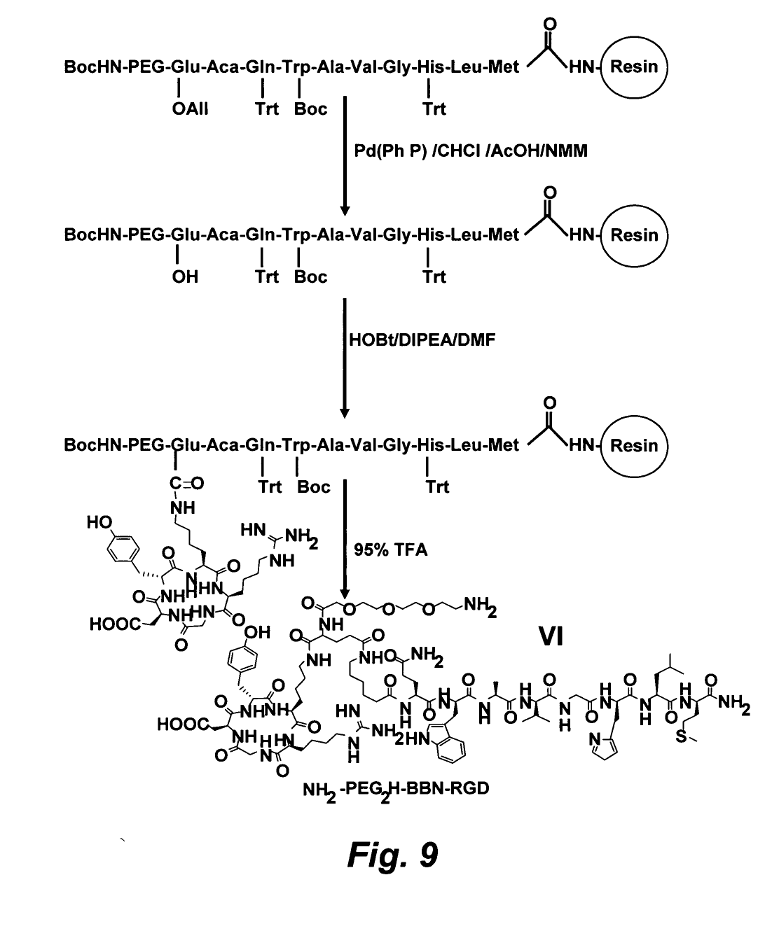

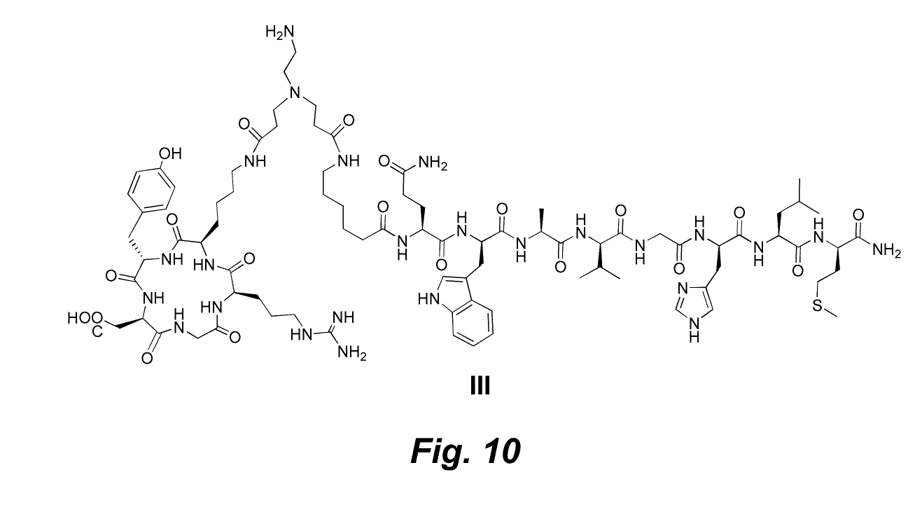

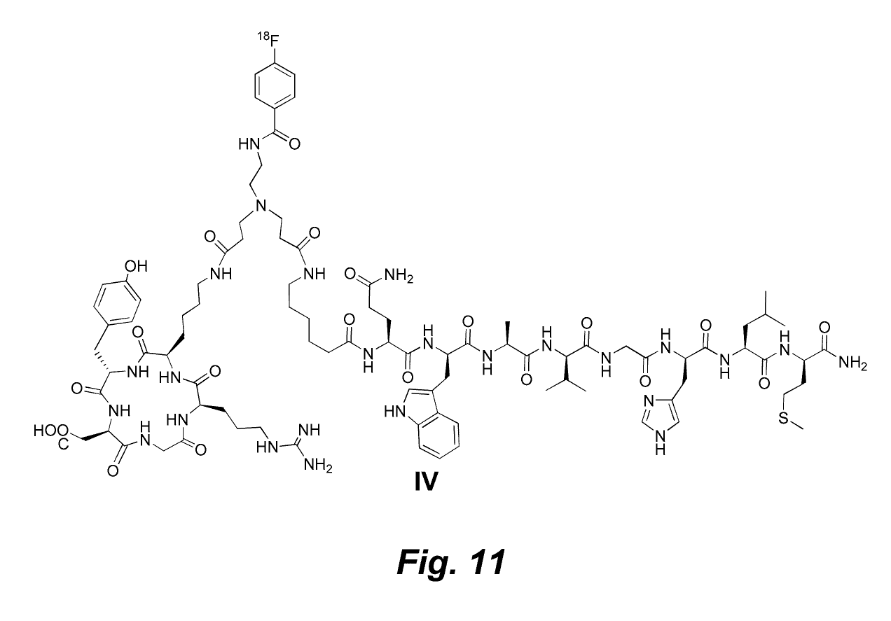

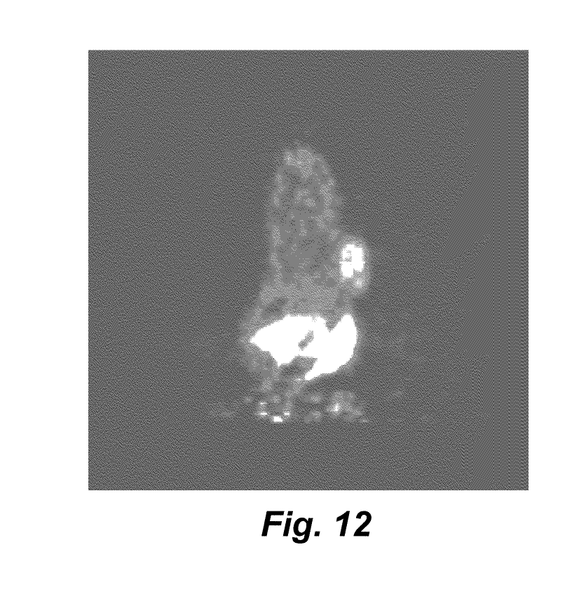

Embodiments of the heterodimeric compositions of the disclosure connected with glutamate are likely to be mixtures of Glu-BBN-RGD (where RGD is on the side-chain 8-position) and Glu-RGD-BBN (where BBN is on the side-chain 8-position), as shown in FIGS. 7A and 7B, that will not be readily separable by HPLC. Synthesis, however, of similar BBN-RGD heterodimers through a symmetric linker or a side-chain-protected glutamic acid can result in just one possible structure. For example, a BBN-RGD heterodimer with a defined structure may be synthesized via a solid-phase synthesis strategy. In this case, Fmoc-Glu-OAII is an orthogonally-protected building block for the synthesis of special peptide sequences. The α-allyl ester can be selectively removed in the presence of Fmoc- and t-Bu-based protecting groups by treatment with Pd(Ph₃P)₄/CHCl₃/HOAc/NMM, thereby facilitating the synthesis of branched amides. As illustrated in FIG. 9, a BBN-RGD heterodimer was synthesized via this solid-phase peptide synthesis strategy. Accordingly, the cyclic RGD peptide c(RGDyK) would be coupled to the α-carboxylate group, and the Aca-BBN(7-14) (FIG. 8) peptide was coupled to the δ-carboxylate group of the glutamate linker. An alternative synthesis is presented in Examples 5-9 below, that can yield the structures as shown in FIGS. 10 and 11.

The radiolabeling yield for the heterodimeric BBN-RGD peptide was found to be lower than that for the monomeric BBN or RGD peptides when using ¹⁸F-SFB as the synthon. There was also a reduced ¹⁸F-labeling yield for an RGD homodimer and a homotetramer in which ¹⁸F-SFB was reacted with the glutamate amine group. While not wishing to be limited to any one theory, the reduced labeling yield may be due to steric hindrance and relatively low reactivity of the glutamate α-amino group. In one embodiment of the disclosure, therefore, and to improve the radiolabeling yield, a mini-PEG linker, 11-amino-3,6,9-trioxaundecanoic acid (NH-mini-PEG-COOH) was introduced to the glutamate residue.

### ⁶⁸Ga-Labeled RGD-BBN Heterodimeric Peptide

The recent introduction of ⁶⁸Ga into clinical practice represents the beginning of the development of a PET imaging probe that is not dependent on the availability of a medical cyclotron. ⁶⁸Ga has the physical property of high positron yield reaching 89% of all disintegrations, which is suitable for PET imaging. Its short physical half-life of 68 min matches the biological half-lives of many peptides and other small molecules owing to their fast blood clearance, quick penetration and rapid target localization. In this study, the in vitro and in vivo characteristics of ⁶⁸Ga-labeled RGD-BBN heterodimeric peptide in a dual integrin- and GRPR-positive PC-3 tumor model were investigated.

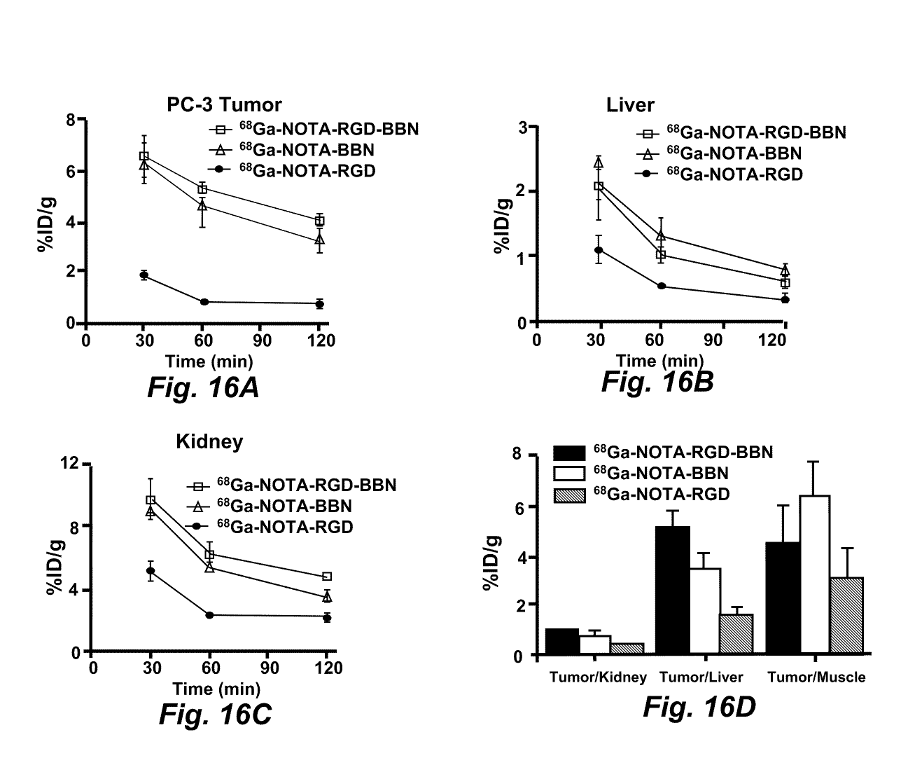

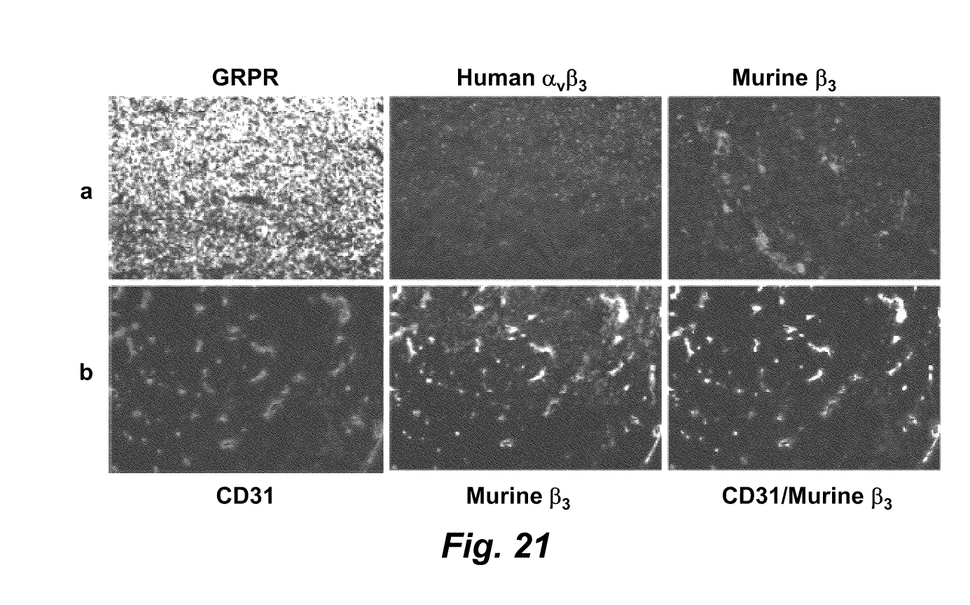

To be a dual functional tracer, each binding motif of the heterodimer must maintain its own function. The receptor binding assay data demonstrated that the binding affinities of RGD-BBN and NOTA-RGD-BBN were similar to that of Aca-BBN(7-14) for GRPR binding and c(RGDyK) for integrin αᵥβ₃ binding, indicating that the RGD-BBN heterodimer can bind both integrin and GRPR in vitro. ⁶⁸Ga-NOTA-RGD-BBN showed lower uptake than ⁶⁸Ga-NOTA-BBN, but higher uptake than 68Ga-NOTA-RGD in PC-3 tumor cells. This may be due to the facts that PC-3 cells have higher numbers of GRPR than integrin, and that the RGD-integrin complex does not tend to internalize into the cells. The internalization of BBN in the RGD-BBN heterodimer was significantly hampered by the recognition of the RGD motif with the cell-surface integrin receptor. The in vivo behavior of ⁶⁸Ga-NOTA-RGD-BBN was tested in a PC-3 tumor model using small-animal PET. The PC-3 tumor uptake of 68Ga-NOTA-RGD-BBN was slightly higher than that of ⁶⁸Ga-NOTA-BBN, but much higher than that of ⁶⁸Ga-NOTA-RGD at all time points examined (FIG. 16A). The higher tumor uptake of ⁶⁸Ga-NOTA-RGD-BBN as compared to ⁶⁸Ga-NOTA-RGD may be explained as PC-3 tumors express relatively high levels of GRPR but low levels of integrin (FIG. 21A). However, the insignificant difference in PC-3 tumor uptake of ⁶⁸Ga-NOTA-RGD-BBN and ⁶⁸Ga-NOTA-BBN seemingly conflicts with the in vitro cell uptake results that ⁶⁸Ga-NOTA-BBN had much higher uptake and retention in PC-3 cells. There are several possible reasons for such an in vitro and in vivo discrepancy. First, in the nude mouse model, the PC-3 tumor vasculature also expresses a high levels of murine integrin 3 (FIG. 21A) which can be recognized by the RGD motif, while the PC-3 tumor cells in vitro do not express murine integrin receptors. The increased integrin receptor numbers may be partially responsible for the slightly increased tumor uptake of ⁶⁸Ga-NOTA-RGD-BBN in vivo. Second, the signal increase of ⁶⁸Ga-NOTA-RGD-BBN over any of the counterpart monomeric tracers may represent a synergistic interaction between the two binding motifs in the heterodimer. It is possible that the binding of one motif, even if only temporarily, could first capture the ⁶⁸Ga-NOTA-RGD-BBN to the target surface or slow down the moving of the ⁶⁸Ga-NOTA-RGD-BBN, allowing the second binding motif to also attach to the tumor, thereby increasing the overall binding and the probability of ⁶⁸Ga-NOTA-RGD-BBN adhering to the tumor. Third, because the RGD motif can recognize murine integrin β3 expressed on the tumor vasculature (FIG. 21B), it is possible that the ⁶⁸Ga-NOTA-RGD-BBN in the circulation would first bind tumor vascular integrin.

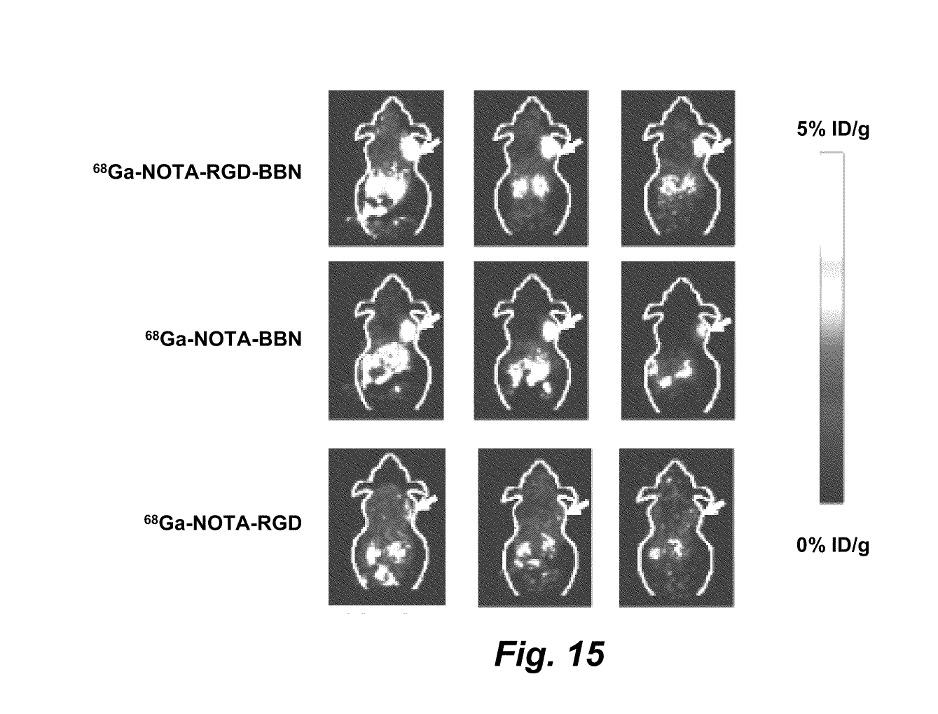

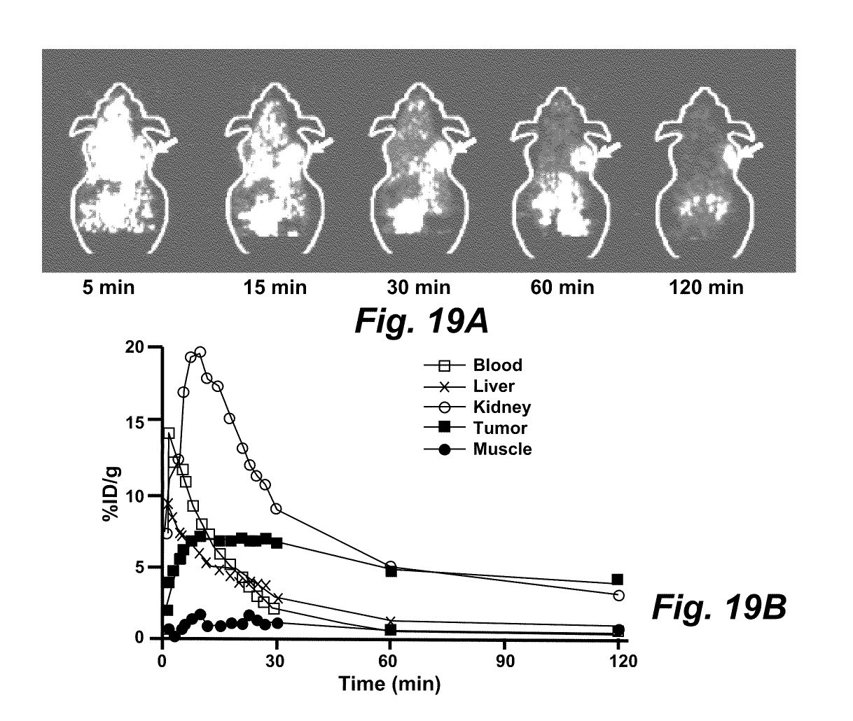

As the binding affinity of RGD monomer is relatively low, the tracer accumulated around the tumor vessel may dissociate from the loosely bound integrin receptors, diffuse into the extracellular matrix and rebind to the tumor cells that express both GRPR and integrin αᵥβ₃. One of the main drawbacks of BBN-based radiotracers is their unfavorable hepatobiliary excretion, which usually results in high intestinal uptake. For example, in this study, as shown in FIG. 15, ⁶⁸Ga-NOTA-BBN exhibited high and persistent intestinal accumulation, which is presumably due to the high lipophilicity of 68Ga-NOTA-BBN. In contrast, ⁶⁸Ga-NOTA-RGD-BBN was excreted mainly through the kidneys as evidenced by the dynamic curve shown in FIG. 19B. The altered in vivo kinetics of ⁶⁸Ga-NOTA-RGDBBN compared with ⁶⁸Ga-NOTA-BBN may be attributed to their differences in molecular size and charge, hydrophilicity, and metabolic stability.

Overall, the heterodimeric tracer significantly reduced the intestinal accumulation of radioactivity, making the tracer more suitable for imaging of abdominal cancer than BBN analogs. The production of ⁶⁸Ga-NOTA-RGD-BBN is easy and does not need an onsite cyclotron, which allows possible kit formulation and widespread availability. The PC-3 tumor uptake of ⁶⁸Ga-NOTA-RGD-BBN (6.55±0.83, 5.26±0.32, and 4.04±0.28% ID/g at 30, 60, and 120 min, respectively) was significantly higher than that of ¹⁸F-FB-PEG3-RGD-BBN (6.35±2.52, 4.41±0.71, and 2.47±0.81% ID/g at 30, 60, and 120 min, respectively) at 60 min after injection (p<0.05). The higher tumor uptake of the ⁶⁸Ga-labeled RGD-BBN is likely due to the internalization and effective trapping of radiometal inside the tumor cells as compared to ¹⁸F-labeled tracers, which is supported by cell efflux studies.

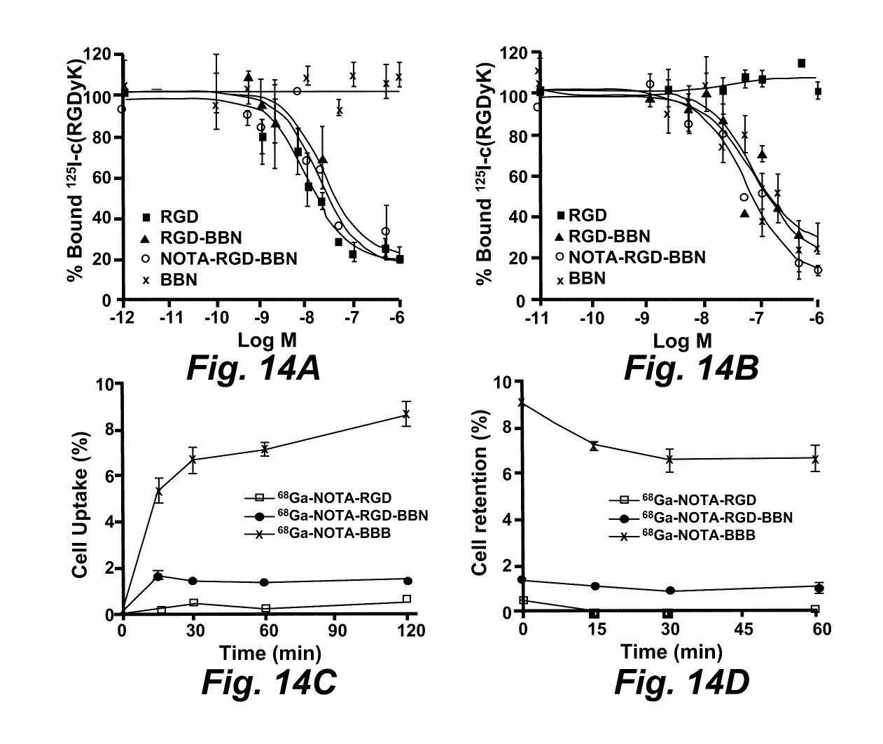

After allowing efflux for 1 h, the efflux ratio was about 40% for ¹⁸F-labeled RGD-BBN, but only about 20% for ⁶⁸Ga-NOTA-RGD-BBN. The cellular uptake of ⁶⁸Ga-NOTA-BBN was much higher than that of ⁶⁸Ga-NOTA-RGD-BBN (FIG. 14D), so cell-trapped ⁶⁸Ga would be higher for ⁶⁸GA-NOTA-BBN than for ⁶⁸Ga-NOTA-RGDBBN. This possibly explains why ⁶⁸Ga-NOTA-BBN also showed much higher tumor uptake than ¹⁸F-labeled BBN tracer. The dual receptor binding specificity of ⁶⁸GA-NOTARGD-BBN in vivo was confirmed by the blocking studies. Either Aca-BBN(7-14) or cyclic RGD peptide c(RGDyK) can only partially inhibit the uptake of ⁶⁸Ga-NOTA-RGD-BBN in the PC-3 tumor, as the BBN motif of the ⁶⁸Ga-NOTA-RGD-BBN can bind to the GRPR when integrin is blocked by RGD, and the RGD motif of the ⁶⁸Ga-NOTA-RGD-BBN can bind to the integrin when GRPR is blocked by BBN.

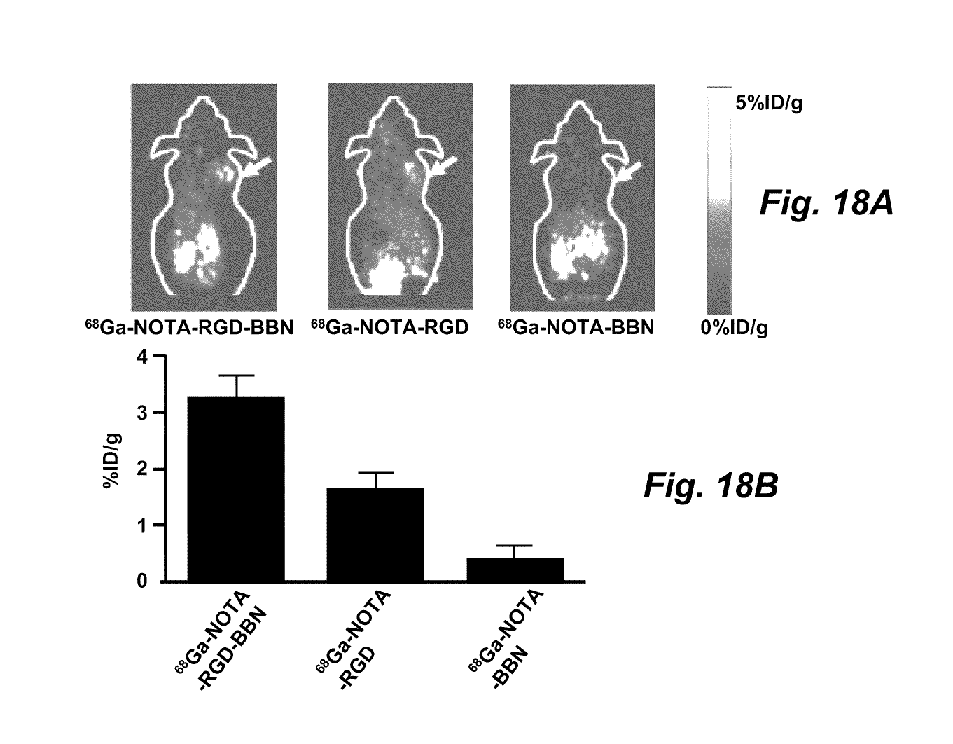

The advantage of dual receptor binding of the heterodimer tracer is apparent when only one receptor type is over-expressed in a tumor model. For example, in the MDA-MB435 tumor model, which expresses a moderate level of integrin αᵥβ₃ but no GRPR, ⁶⁸Ga-NOTA-BBN was unable to detect the tumors because it only recognizes GRPR. In contrast, ⁶⁸Ga-NOTA-RGD and ⁶⁸Ga-NOTA-RGD-BBN had a clear tumor uptake due to the function of RGD (FIGS. 18A and 18B).

The MDA-MB-435 tumor uptake of ⁶⁸Ga-NOTA-RGD-BBN was even higher than that of ⁶⁸Ga-NOTA-RGD tumor, which may have resulted from the improved in vivo kinetics and increased circulation half-life of ⁶⁸Ga-NOTA-RGD-BBN over ⁶⁸Ga-NOTA-RGD. In the ⁶⁸Ga-NOTA-RGD-BBN heterodimeric peptide, the RGD and BBN motifs were linked through a glutamic acid. Due to the short length of the linker, it is impossible for the RGD and BBN motifs to bind both integrin and GRPR simultaneously. Therefore, in future it would be interesting to investigate the effects of linkers of different lengths, solubility, lipophilicity, and flexibility on the in vitro and in vivo behaviors of the heterodimeric peptides. The design of heteromultimeric tracers that recognize other tumor targets is also worth further investigation for tumor-targeted imaging and therapy. In conclusion, we have described the design and synthesis of ⁶⁸Ga-labeled RGD-BBN heterodimer peptide containing both RGD and BBN motifs for dual integrin and GRPR-targeted tumor imaging. ⁶⁸Ga-NOTA-RGD-BBN exhibited dual receptor targeting properties both in vitro and in vivo. The high affinity and specificity and improved pharmacokinetics of the ⁶⁸Ga-labeled RGD-BBN heterodimer make it a promising agent for molecular imaging of tumors with both or either receptor expression pattern. The heterodimer and heteromultimer strategy may also provide general methods of developing tumor-targeted imaging probes and therapeutic agents.

⁶⁴Cu-Labeled RGD-BBN heterodimeric Peptide

1,4,7,10-Tetraazacyclododecane-N,N9,N99,N999-tetraacetic acid (DOTA) is a known bifunctional chelators for ⁶⁴Cu labeling. However, the relatively low thermodynamic and kinetic stability of ⁶⁴Cu-DOTA in vivo is well documented (Wadas et al., (2008). J. Nucl. Med.; 49: 1819-1827; Prasanphanich et al., (2007) Proc. Natl. Acad. Sci. USA 104: 12462-12467; Boswell et al., (2004) J. Med. Chem. 47: 1465-1474; Garrison et al., (2007) J. Nucl. Med. 48: 1327-1337). The instability of the ⁶⁴Cu-DOTA conjugates results in demetallation and subsequent accumulation in non-target tissues such as liver (Prasanphanich et al., (2007) Proc. Natl. Acad. Sci. USA 104: 12462-12467). Prasanphanich et al. recently reported ⁶⁴Cu-labeled BBN analogs using 1,4,7-triazacyclononane-1,4,7-triacetic acid (NOTA) as a chelator. The results suggested high in vivo kinetic stability of ⁶⁴Cu-NOTA-BBN vectors with little or no dissociation of ⁶⁴Cu from NOTA (Prasanphanich et al., (2007) Proc. Natl. Acad. Sci. USA 104: 12462-12467). The present disclosure provides data for the advantages of ⁶⁴Cu-labeled NOTA-RGD-BBN heterodimer over its monomeric counterparts NOTA-RGD and NOTA-BBN for imaging GRPR-positive tumors, and also compare the in vitro and in vivo characteristics of ⁶⁴Cu-labeled RGD-BBN heterodimer using NOTA as a chelator with those using DOTA as a chelator. The present disclosure further provides methods for using ⁶⁴Cu-NOTARGD-BBN to image tumors that express integrin but not GRPR (e.g., 4T1 murine mammary carcinoma). The synergistic effects of the heterodimer, RGD-BBN was shown with labeling ⁶⁴Cu (t_{1/2}=12.7 h) using DOTA (1,4,7,10-tetraazacyclododecane-1,4,7,10-tetraacetic acid) and NOTA (1,4,7-triazacyclononane-1,4,7-triacetic acid) as the chelator, respectively. The in vitro and in vivo characteristics of ⁶⁴Cu-NOTA-RGD-BBN were compared with ⁶⁴Cu-NOTA-RGD, ⁶⁴Cu-NOTA-BBN, and ⁶⁴Cu-DOTA-RGD-BBN. The dual receptor targeting properties of ⁶⁴Cu-NOTA-RGD-BBN was also investigated in tumor models.

⁶⁴Cu-NOTA-RGD-BBN and ⁶⁴Cu-DOTA-RGD-BBN had comparable dual integrin αᵥβ₃ and GRPR-binding affinities, but their affinities were both slightly lower than that of RGD and BBN respectively. ⁶⁴Cu-NOTA-RGD-BBN possessed significantly higher tumor uptake compared with ⁶⁴Cu-NOTA-RGD, ⁶⁴Cu-NOTA-BBN, the mixture of ⁶⁴Cu-NOTA-RGD+⁶⁴Cu-NOTA-BBN, and also ⁶⁴Cu-DOTA-RGD-BBN. ⁶⁴Cu-NOTA-RGD-BBN also showed improved in vivo kinetics such as lower liver and intestine activity accumulation than the BBN tracers. The synergistic effects of ⁶⁴Cu-NOTA-RGD-BBN were observed in both the dual receptor-positive PC-3 tumor model and one receptor-positive 4T1 tumor model.

⁶⁴Cu has favorable decay characteristics (half-life, 12.7 h; b1, 17.8%; b2, 38.4%), making it useful for both PET and internal radiotherapy. ⁶⁴Cu can be produced in high yield and at high specific activity on a small biomedical cyclotron and is labeled with biomolecules through macrocyclic chelators, which allow possible kit formulation and wide availability. More important, the longer half-life of ⁶⁴Cu among all the positron emitters allows imaging at late time points to acquire more in vivo information than is possible for ¹⁸F (half-life, 109.7 min).

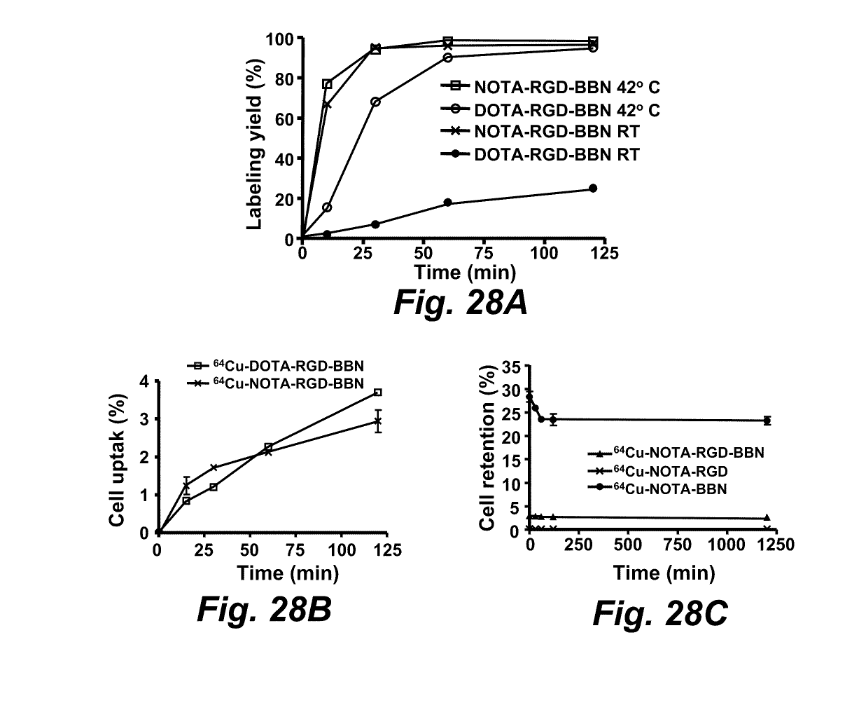

Both DOTA and NOTA can be used as bifunctional chelators for ⁶⁴Cu labeling. The ⁶⁴Cu-DOTA conjugates usually exhibit a high accumulation of liver radioactivity because of the dissociation of ⁶⁴Cu in vivo from DOTA, followed by metabolism and transchelation to other proteins. NOTA is most commonly used for ⁶⁸Ga (half-life, 68 min) labeling because the rapid reaction kinetics of NOTA match the short half-life of ⁶⁸Ga. NOTA was also reported to be labeled with ⁶⁴Cu, with reduced liver accumulation. In the present disclosure, DOTA-RGD-BBN and NOTA-RGD-BBN were synthesized and labeled both conjugates with ⁶⁴Cu. Compared with DOTA-RGD-BBN, NOTA-RGD-BBN can be more easily labeled with ⁶⁴Cu, as shown in FIG. 28A. The direct in vivo comparison of ⁶⁴Cu-NOTA-RGD-BBN and ⁶⁴Cu-DOTA-RGD-BBN showed the former to have much higher tumor uptake and tumor to non-tumor ratios and lower liver uptake than the latter.

⁶⁴Cu-CB-TE2A-8-AOC-BBN(7-14)NH₂ showed significant improvement in clearance because of its improved in vivo stability, compared with the DOTA conjugates. Most important, the liver uptake of the CB-TE2A conjugate was also significantly lower than that of the DOTA conjugates. Compared with ⁶⁴Cu-CB-TE2A-8-AOC-BBN(7-14)NH₂, 64Cu-NOTA-RGD-BBN showed slightly higher liver uptake, but the tumor uptake of ⁶⁴Cu-NOTA-RGD-BBN was also higher than that of ⁶⁴Cu-CB-TE2A-8-AOC-BBN(7-14)NH₂. Radiolabeling of CB-TE2A conjugates requires harsher reaction conditions than does radiolabeling of DOTA and NOTA conjugates. The high temperature and high pH required for CB-TE2A labeling may not be suitable for peptides such as RGD-BBN. In contrast, the fast reaction kinetics of NOTA-conjugates would be more suitable for clinical translation. The in vivo behaviors of the ⁶⁴Cu-labeled NOTA conjugates were compared in the PC-3 tumor model. Because of the high GRPR and low integrin αᵥβ₃ expression of the PC-3 tumor, tumor uptake of ⁶⁴Cu-NOTA-RGD was low and the ⁶⁴Cu-NOTA-BBN showed relatively high tumor contrast. However, high accumulation of radioactivity in the abdominal region, especially in the intestines, was observed in the mice receiving ⁶⁴Cu-NOTA-BBN and other reported BBN tracers, suggesting hepatobiliary excretion of ⁶⁴Cu-NOTA-BBN. In contrast, ⁶⁴Cu-NOTA-RGD-BBN showed much lower intestinal accumulation, and the tumor uptake of ⁶⁴Cu-NOTA-RGDBBN was also significantly higher than that of ⁶⁴Cu-NOTA-RGD, ⁶⁴Cu-NOTA-BBN, and ⁶⁴Cu-NOTA-RGD plus ⁶⁴Cu-NOTA-RGD. The high lipophilicity of ⁶⁴Cu-NOTA-BBN resulted in rapid hepatobiliary excretion, which led to a short circulation half-life for the tracer, and thus insufficient time for the tracer to extravasate from the tumor blood vessels, diffuse in the extracellular space, and bind with GRPR expressed on the tumor cells. In contrast, ⁶⁴Cu-NOTA-RGD-BBN had longer blood retention and predominantly renal clearance.

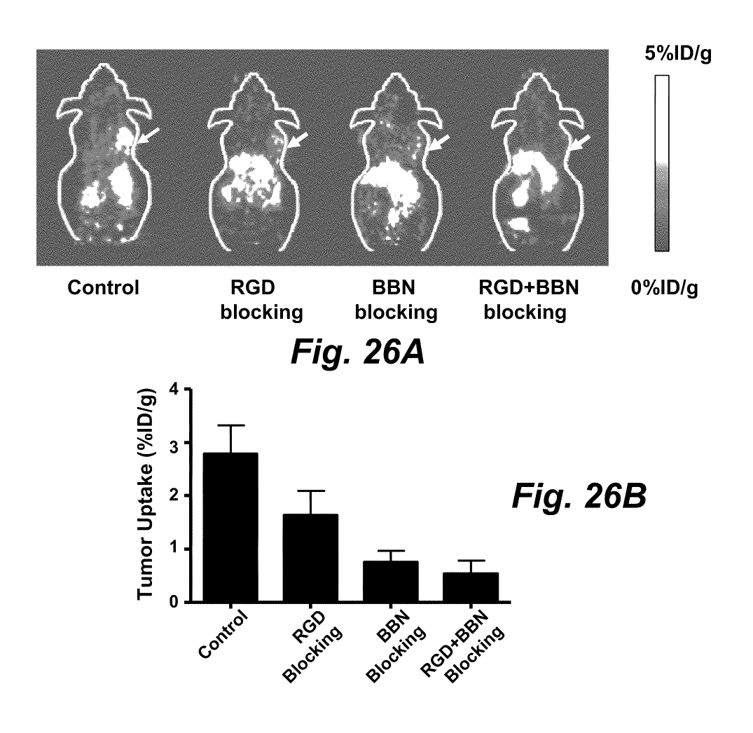

Another major reason for the higher tumor uptake of ⁶⁴Cu-NOTA-RGD-BBN than of ⁶⁴Cu-NOTA-BBN is the dual GRPR- and integrin-targeting properties of the RGD-BBN heterodimer (FIG. 26A). Although tumor uptake of RGD alone is low, binding of the RGD motif in the RGD-BBN heterodimer molecule with integrins expressed on the tumor vasculature would significantly increase the local concentration of peptide in the tumor, facilitating the binding of BBN with GRPR on tumor cells around the blood vessels. This may also explain why tumor uptake of ⁶⁴Cu-NOTA-RGD-BBN was much higher than that of the co-injection of ⁶⁴Cu-NOTA-RGD plus ⁶⁴Cu-NOTA-BBN, as was also found in the case of ₁₈F-FB-PEG3-RGD-BBN.

The dual-receptor targeting of ⁶⁴Cu-NOTA-RGD-BBN may also contribute to prolonged tumor retention of the tracer. For example, at 24 h after injection, tumor uptake of ⁶⁴Cu-NOTA-RGD-BBN was 2.04±0.35% ID/g, which is significantly higher than that of the corresponding ⁶⁴Cu-NOTA-BBN (0.44±0.39% ID/g).

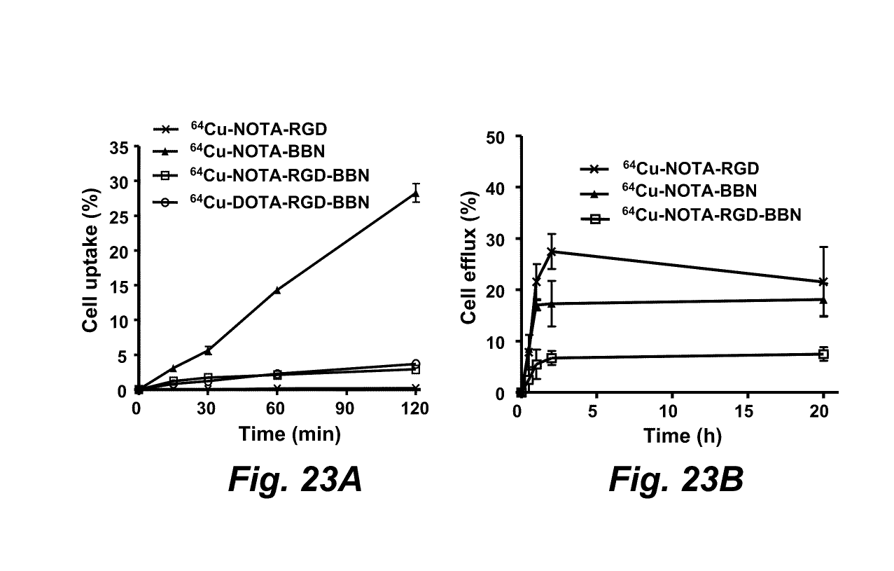

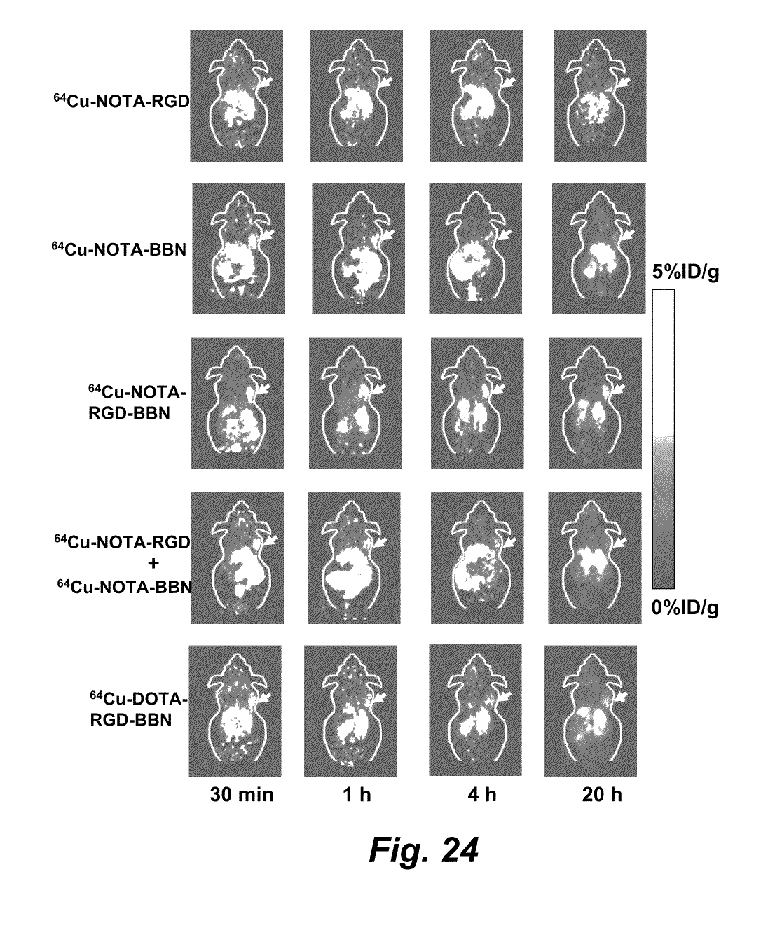

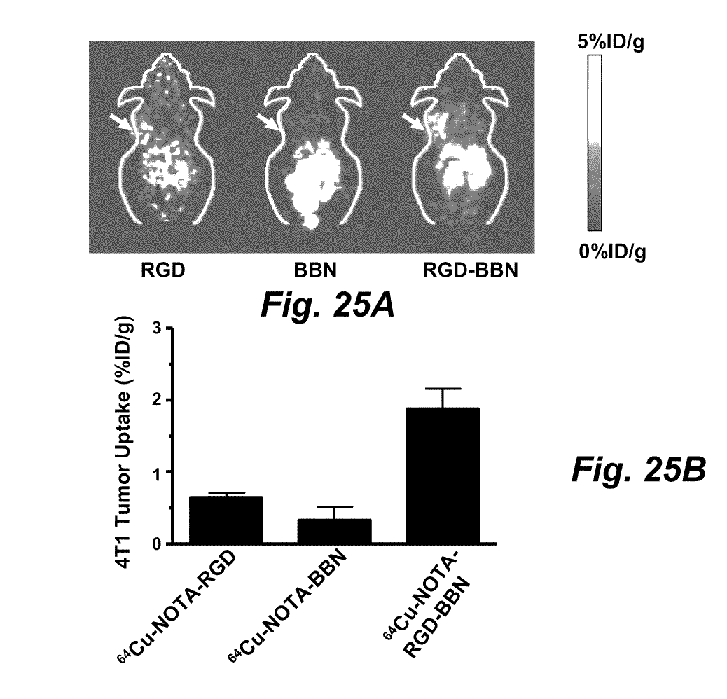

Tumor retention of ⁶⁴Cu-NOTA-RGD-BBN was also higher than that of the ⁶⁴Cu-labeled NOTA conjugated BBN tracers reported by Prasanphanich et al. The prolonged tumor retention of ⁶⁴Cu-NOTA-RGD-BBN, compared with that of ⁶⁴Cu-NOTA-BBN, was consistent with the in vitro findings that the efflux ratio of ⁶⁴Cu-NOTA-RGD-BBN was much lower than that of the BBN tracer (FIG. 23). The PET images of mice that received ⁶⁴Cu-NOTA-RGD plus ⁶⁴Cu-NOTA-BBN represented almost a merge of the images acquired after injection of ⁶⁴Cu-NOTA-RGD and ⁶⁴Cu-NOTA-BBN alone (FIG. 24). For example, ⁶⁴Cu-NOTA-RGD showed clear kidney uptake and ⁶⁴Cu-NOTA-BBN showed predominantly intestinal accumulation, whereas the co-injection of ⁶⁴Cu-NOTA-RGD and ⁶⁴Cu-NOTA-BBN exhibited both renal and abdominal uptake (FIG. 24). The reduced liver and intestinal accumulation and increased tumor uptake of ⁶⁴Cu-NOTA-RGDFIGURE BBN, compared with ⁶⁴Cu-NOTA-RGD plus ⁶⁴Cu-NOTA-BBN, clearly demonstrates that ⁶⁴Cu-NOTA-RGD-BBN is superior to the sum of ⁶⁴Cu-NOTA-RGD and ⁶⁴Cu-NOTA-BBN. The advantage of ⁶⁴Cu-NOTA-RGD-BBN over ⁶⁴Cu-NOTA-RGD and ⁶⁴Cu-NOTA-BBN was also confirmed in a 4T1 tumor model that is only integrin-positive. ⁶⁴Cu-NOTA-BBN was unable to detect 4T1 tumor because of the lack of GRPR expression. In contrast, both ⁶⁴Cu-NOTA-RGD and ⁶⁴Cu-NOTA-RGD-BBN showed tumor contrast (FIGS. 25A and 25B). Uptake of ⁶⁴Cu-NOTA-RGD-BBN was even higher than that of ⁶⁴Cu-NOTA-RGD in 4T1 tumor, possibly as a result of the improved in vivo kinetics and increased circulation retention of ⁶⁴Cu-NOTA-RGD-BBN over ⁶⁴Cu-NOTA-RGD.

⁶⁴Cu-NOTA-RGD-BBN had blood and kidney clearance curves comparable to those of ¹⁸F-PEG3-RGD-BBN tracer in the first hour after injection, but between 1 and 2 h, the ¹⁸F tracer cleared more rapidly, possibly because of the higher hydrophilicity of ¹⁸F-PEG3-RGD-BBN. The PC-3 tumor uptake of ¹⁸F-PEG3-RGD-BBN was also significantly higher than that of ⁶⁴Cu-NOTA-RGD-BBN at 30 min (6.35±2.52 vs. 3.06±0.11% ID/g) and 1 h (4.41±0.71 vs. 2.78±0.56% ID/g).

Although liver uptake of ⁶⁴Cu-NOTARGD-BBN was relatively low (3.5% ID/g at any time point tested), it was still higher than that of ¹⁸F-PEG3-RGD-BBN. Taken together, ¹⁸F-PEG3-RGD-BBN is better than ⁶⁴Cu-NOTA-RGD-BBN for tumor imaging within 2 h after injection. However, because of the short half-life of ¹⁸F, the absolute tumor signal of ¹⁸F-PEG3-RGD-BBN was low after 2 h. In contrast, the plateau in tumor uptake of ⁶⁴Cu-NOTA-RGD-BBN from 4 to 20 h allows a persistent imaging signal. More important, because of the decay characteristics of ⁶⁴Cu, the longer tumor retention of ⁶⁴Cu-NOTA-RGD-BBN makes possible GRPR-positive tumor-targeted therapy.

### Breast Cancer Imaging

Breast cancers can be sorted into two categories, estrogen dependent (ER⁺) and estrogen-independent (ER⁻), based on the presence or absence of estrogen receptors (Vaïk et al., (2009) Proteomics Clin Appl 3: 41-50). Nowadays, many ER⁺ and ER⁻ tumor cells are being used for breast cancer research in animal studies. We screened the GRPR and integrin αᵥβ₃ expression in both the ER⁺ (T47D, BT474, MCF-7) and ER⁻ (MDA-MB-231, MDA-MB-435, MDA-MB468, BT20) breast cancer cells (Cassoni et al., (2001) J. Clin. Endocrinol. Metab. 86: 1738-1745; Bajo et al., (2002) Proc. Natl. Acad. Sci. USA 99: 3836-3841; Anzick et al., (1997) Science 277: 965-968; Brandi et al., (2003) Cancer Res 63: 40284036).

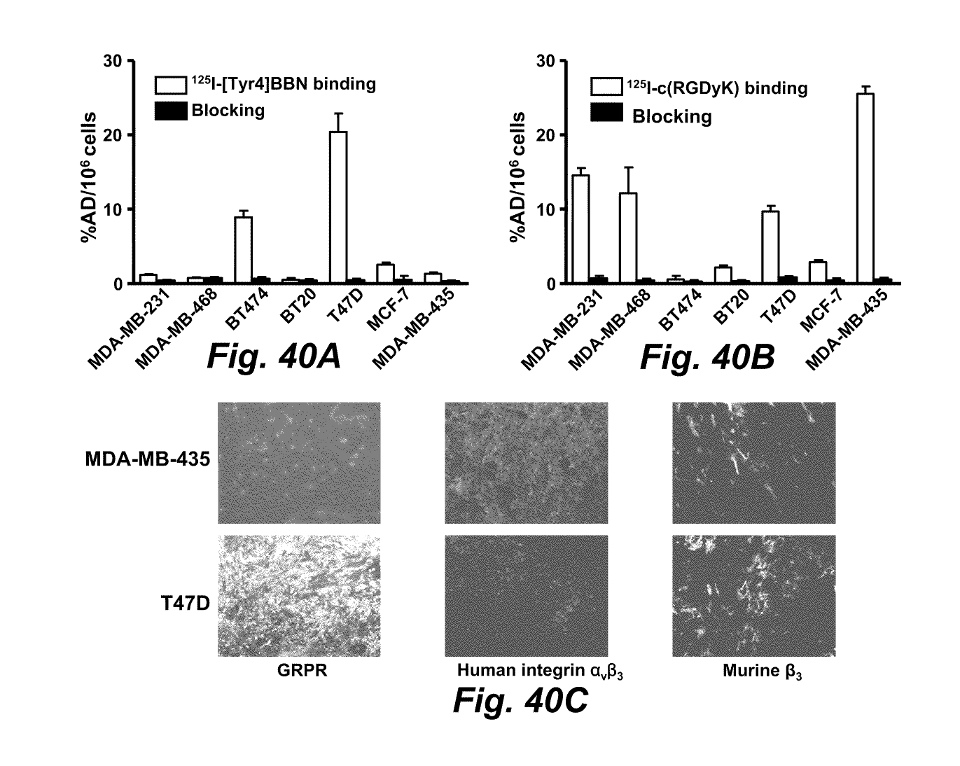

The GRPR expression on estrogen-dependent tumor cells such as T47D, BT474 was high, but the integrin αᵥβ₃ expression was relatively low or moderate. However, the estrogen-independent tumor cells such as MDA-MB435, MDA-MB-231, MDA-MB-468 expressed higher integrin αᵥβ₃, but their GRPR expression was undetectable (FIGS. 40A-C). T47D and MDA-MB-435 tumor cells were selected for further investigation, which represent the two types of breast cancers. The tumor tissues were investigated by immunohistochemical staining to conform the receptors expression. The expression of GRPR and human integrin αᵥβ₃ was consistent with the cell binding assay data. Because the murine endothelial cells would also involved in the growth of the human tumor xenografts inoculated in the mice, so the murine integrin β₃ was also detected to be positive in both the T47D and MDA-MB435 tumor tissues, as shown in FIG. 40C.

The in vivo behaviors of the three tracers were tested by microPET in T47D and MDA-MB435 orthotopic breast cancer models. All the tracers showed contrast tumor imaging in the two tumor models from 30 min p.i. The radiolabeled BBN was also tested in the MDA-MB-435 tumor model that did not express GRPR for control studies. The much higher tumor uptake of the ¹⁸F, ⁶⁴Cu, or ⁶⁸Ga labeled RGD-BBN tracer than that of the corresponding BBN tracer indicated that the RGD-BBN tracers were useful to detect the tumor with only one receptor positive, but the BBN tracers can only be used for GRPR-positive tumor imaging.

The present disclosure provides data that demonstrate that ¹⁸F, ⁶⁴Cu, and ⁶⁸Ga labeled RGD-BBN heterodimeric peptides can be used to detect both the GRPR⁺/(integrin αᵥβ₃ low expression) and GRPR⁻/integrin αᵥβ₃⁺ breast cancers by microPET imaging. Although ¹⁸F-labeled RGD-BBN showed lower tumor uptake than ⁶⁴Cu-NOTA-RGD-BBN and ⁶⁸Ga-NOTA-RGD-BBN, it was able to detect breast cancer tumors in xenograft models with high contrast and low background. However, the preparation of the ¹⁸F-FB-PEG₃-RGD-BBN was more complex and time-consuming. Synthesis of ⁶⁴Cu-NOTA-RGD-BBN and ⁶⁸Ga-NOTA-RGD-BBN is faster, which allows kit formulation and wide availability.

⁶⁴Cu-NOTA-RGD-BBN showed prolonged tumor uptake, but also higher liver retention and kidney uptake. Modification of the ⁶⁴Cu-chelator system would be the future focus to develop a superior RGD-BBN radiotracer for GRPR and integrin targeting and possible internal radiotherapy. ⁶⁸Ga-NOTA-RGD-BBN possessed high tumor signals, but also high background uptake. The insertion of hydrophilic linkers such as PEG₃ between the RGD-BBN and NOTA may be applied for future development of ⁶⁸Ga labeled RGD-BBN tracer with low background signals for breast cancer imaging.

It is further contemplated that the heterodimeric compositions according to the present disclosure may be suitable as carriers to transport a non-labeling agent, such as a therapeutic agent, to a target cell having a combination of cell-surface exposed GPRP and integrin molecules. Embodiments of the heterodimeric probes of the disclosure may, therefore, further comprise covalently bound agents including, but not limited to, cytotoxic agents, cell proliferation modulating agents and the like that may be attached to an exposed side-group of the linker of a domain of the probe construct. It is further contemplated that embodiments of the heterodimeric probes of the present disclosure may comprise both a labeled prosthetic group and a non-labeled prosthetic group such as a therapeutic agent, a radionuclide, or the like such that the site of delivery of the non-labeled group may be imaged. Furthermore, it is contemplated that embodiments of the heterodimeric probes of the present disclosure may comprise more than one labeled prosthetic group, whereby more than one detection technique may be used to determine the location of the probe within a cell or whole animal. For example, but not limiting, an F-18 label for PET scanning and a fluorescent fluorophore may be tethered to the heterodimeric probe.

One aspect of the disclosure, therefore, provides compositions that can comprise a heterodimeric probe, where the heterodimeric probe comprises: a first peptide domain comprising a moiety capable of selectively binding to an integrin; a second peptide domain comprising a moiety capable of selectively binding to a gastrin-releasing peptide receptor; a linker connecting the first peptide domain and the second peptide domain; and a prosthetic group.

In embodiments of this aspect of the disclosure, the first peptide domain may comprise at least one tripeptide comprising the amino acid sequence arginine-glycine-aspartate (Arg-Gly-Asp).

In embodiments of this aspect of the disclosure, the moiety capable of selectively binding to an integrin may comprise at least one peptide selected from the group consisting of: cyclo(Arg-Ala-Asp-D-Phe-Lys), cyclo(Arg-Ala-Asp-D-Phe-Val), cyclo(Arg-Ala-Asp-D-Tyr-Cys), cyclo(Arg-Ala-Asp-D-Tyr-Lys), cyclo(Arg-Gly-Asp-D-Phe-Cys), cyclo(Arg-Gly-Asp-D-Phe-Glu), cyclo(Arg-Gly-Asp-D-Phe-Lys), cyclo(Arg-Gly-Asp-D-Tyr-Cys), cyclo(Arg-Gly-Asp-D-Tyr-Glu), cyclo(Arg-Gly-Asp-D-Tyr-Lys), cyclo[Arg-Gly-Asp-D-Phe-Lys(Ac-SCH₂CO)], cyclo[Arg-Gly-Asp-D-Phe-Lys(H-Ser)], cyclo[Arg-Gly-Asp-D-Phe-Lys(PEG-PEG)], H-Glu[cyclo (Arg-Gly-Asp-D-Phe-Lys)]₂, H-Glu[cyclo(Arg-Gly-Asp-D-Phe-Lys)]₂, H-Glu[cyclo(Arg-Gly-Asp-D-Tyr-Lys)]₂, H-Gly-Arg-Ala-Asp-Ser-Pro-OH (SEQ ID NO.: 1), H-Gly-Arg-Gly-Asp-Asn-Pro-OH (SEQ ID NO.: 2), H-Gly-Arg-Gly-Glu-Ser-OH (SEQ ID NO.: 3), cyclo(Arg-Gly-Asp-D-Phe-Lys), H-Arg-Gly-Asp-Ser-Lys-OH (SEQ ID NO.: 4), H-Arg-Ala-Asp-Ser-Lys-OH (SEQ ID NO.: 5), Ac-Gly-D-Arg-Gly-Asp-Ser-Pro-Ala-Ser-Ser-Lys-(Gly)-4-Ser-D-Arg-(Leu)-6-D-Arg-NH₂, cyclo(Arg-Gly-Glu-D-Phe-Lys), and cyclo(Arg-Gly-Asp-D-Phe-Val).

In some embodiments of this aspect of the disclosure, the moiety capable of selectively binding to an integrin may comprise cyclo(arginine-glycine-aspartate-D-tyrosine-lysine).

In embodiments of this aspect of the disclosure, the first peptide domain may comprise a multimer of conjugated peptides, wherein at least one peptide of the multimer of peptides comprises the amino acid sequence arginine-glycine-aspartate.

In some embodiments of this aspect of the disclosure, the amino acid sequence of each peptide of the multimer of peptides may comprise the amino acid sequence of arginine-glycine-aspartate.

In one embodiment of this aspect of the disclosure, at least one peptide of the multimer of peptides comprises cyclo(arginine-glycine-aspartate-D-tyrosine-lysine).

In embodiments of this aspect of the disclosure, the moiety capable of selectively binding to a gastrin-releasing peptide receptor may comprise a fragment of the polypeptide bombesin, wherein the fragment has an affinity for a gastrin-releasing peptide receptor.

In embodiments of this aspect of the disclosure, the moiety capable of selectively binding to a gastrin-releasing peptide receptor can be selected from the group consisting of: bombesin(7-14) having the amino acid sequence of glutamine-tryptophan-alanine-valine-glycine-histidine-leucine-methionine (SEQ ID NO: 6), bombesin(8-14) having the amino acid sequence of asparagine-glutamine-tryptophan-alanine-valine-glycine-histidine-leucine-methionine (SEQ ID NO: 7), [Lys³]BBN (SEQ ID NO.: 8), [(D)Phe⁶, Leu-NHEt¹³, des-Met¹⁴]BN(6-14), (H-(D)Phe-Gln-Trp-Ala-Val-Gly-His-Leu-NHEt, or substituted variants thereof, wherein the substituted variants have an affinity for a GRPR.

In embodiments of this aspect of the disclosure, the second domain is bombesin(7-14) and comprises the amino acid sequence of glutamine-tryptophan-alanine-valine-glycine-histidine-leucine-methionine (SEQ ID NO.: 6). In these embodiments of this aspect of the disclosure, the heterodimer probe selectively binds to the integrin (αᵥβ₃.

In embodiments of this aspect of the disclosure, the heterodimer probe may selectively bind to the integrin αᵥβ₃ and gastrin-releasing peptide receptor.

In embodiments of this aspect of the disclosure, the linker connecting the first peptide domain and the second peptide domain may comprise the formula (HOOC)—(CH₂)ₙ—(CHNH₂.)—(CH₂)ₘ—(COOH)ₐ, wherein n and m are each independently 0, or an integer from 1 to about 10, and a is an integer from 1 to about 10.

In embodiments of this aspect of the disclosure, the linker connecting the first peptide domain and the second peptide domain can be selected from the group consisting of (aspartate)ₓ, (glutamate)_{y}, wherein x and y are each independently integers from 1 to about 10, or any combination thereof.

In some embodiments of this aspect of the disclosure, the linker connecting the first peptide domain and the second peptide domain is a glutamate residue or an aspartate residue.

In embodiments of this aspect of the disclosure, the linker may further comprise a tether covalently bound thereto, and wherein the tether is between the linker and the prosthetic group. In these embodiments of this aspect of the disclosure, the tether between the linker comprises (Gly)ₙ, wherein n is an integer from 1 to about 12.

In some embodiments of this aspect of the disclosure, the tether may further comprise at least one polyethylene glycol moiety, and wherein the polyethylene glycol moiety has a molecular weight of about 200 to about 5000 daltons.

In one embodiment of this aspect of the disclosure, the tether is a polyethylene glycol-3 (11-amino-3,6,9,-trioxaundecanoate moiety.

In embodiments of this aspect of the disclosure, the prosthetic group comprises one of: a detectable label, a therapeutic agent, a reactive group capable of covalently bonding to a detectable label or a therapeutic agent, or a combination thereof.

In embodiments thereof, the prosthetic group may comprise a detectable label, or a group capable of bonding to a detectable label.

In embodiments of this aspect of the disclosure, the group capable of bonding to a detectable label can be selected from an amine group, a carboxyl group, and metal chelating group.

In some embodiments of this aspect of the disclosure, the metal chelating group is NOTA (1,4,7-triazacyclononane-1,4,7-triacetate) or DOTA (1,4,7,10-tetraazacyclododecane-N,N′,N″,N′″-tetracetate).

In embodiments of this aspect of the disclosure, the prosthetic group may comprise a radiolabel, an optical label, or a radiolabel suitable for radiotherapy. In these embodiments of this aspect of the disclosure, the prosthetic group may comprise a detectable label selected from the group consisting of: the fluoride isotope ¹⁸F, ⁶⁸Ga, ⁶⁴Cu, ⁸⁶Y, ¹²⁴I, ¹¹¹In, ^{99m}Tc, ^{123/131}I, a fluorescent dye, a quantum dot, an alpha emitter, a beta emitter, and a gamma emitter.

In some embodiments of this aspect of the disclosure, the prosthetic group comprises a radionuclide selected from the group consisting of ¹⁸F, ⁶⁸Ga, and ⁶⁴Cu.

In one embodiment of this aspect of the disclosure, the prosthetic group is ¹⁸F-fluorobenzoate.

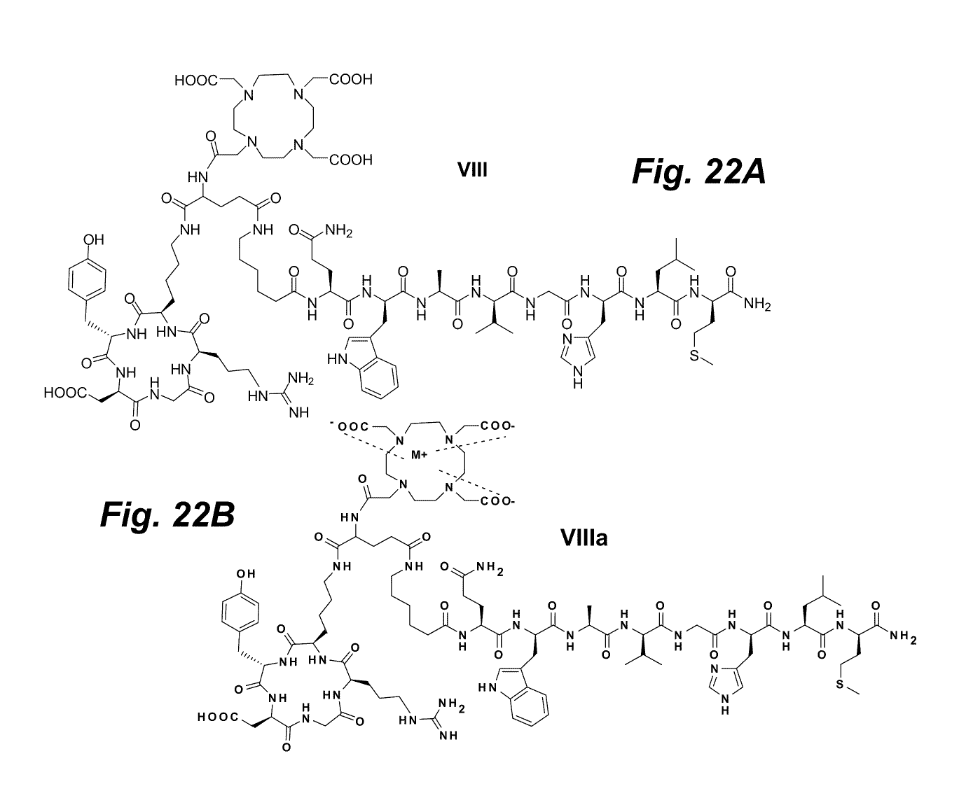

In some embodiments of this aspect of the disclosure, the heterodimer has a formula selected from the group consisting of: I, II, III, IV, V, VI, VII, VIII, VIIIa, and IX, as shown in FIGS. 7B, 1B, 10, 11, 7A, 9, 13, 22A, 22B, and 33 respectively, wherein formula VIIIa comprises a metal ion M+. In some embodiments of this aspect of the disclosure, the metal ion M+ is ⁶⁸Ga or ⁶⁴Cu.

In embodiments of this aspect of the disclosure, the compositions may further comprise a pharmaceutically acceptable carrier.

Another aspect of the present disclosure encompasses methods of identifying a cell or a population of cells expressing an integrin and a gastrin-releasing peptide receptor, comprising: contacting a cell or population of cells with a composition according to any of the above embodiments, the composition comprising a heterodimeric probe capable of selectively binding to an integrin and to a gastrin-releasing peptide receptor of a cell; allowing the heterodimeric polypeptide probe to selectively bind to at least one of an integrin and to a gastrin-releasing peptide receptor of a cell or a population of cells; and detecting the presence of the heterodimeric probe on the cell or population of cells, whereby the presence of the heterodimeric probe on the cell or population of cells indicates that the cell or population of cells has an integrin, a gastrin-releasing peptide receptor, or both an integrin and a gastrin-releasing peptide receptor thereon.

In embodiments of this aspect of the disclosure, the cell or population of cells may be mammalian cells, and the cells or population of cells may be isolated cells.

In other embodiments of this aspect of the disclosure, the cell or population of cells comprise mammalian cells, and wherein the cells or population of cells are in a tissue of a human or animal host.

In embodiments of this aspect of the disclosure, the heterodimer probe may bind to the integrin αᵥβ₃, the gastrin-releasing peptide receptor, or the combination thereof.

In embodiments of this aspect of the disclosure, the composition may comprise the heterodimeric probe is administered to an animal or human host.

In embodiments of this aspect of the disclosure, the heterodimer has a formula selected from the group consisting of: I, II, IV, VII, VIIIa, and IX, wherein, M+ can be a radionuclide selected from ⁶⁸Ga and ⁶⁴Cu, and as shown in FIGS. 7B, 1B, 11, 13, 22B, and 33 respectively.

In embodiments of this aspect of the disclosure, the heterodimeric probe can be detected by positron emission tomography or by single photon emission computed tomography.

In embodiments of this aspect of the disclosure, the heterodimeric probe may be admixed with a pharmaceutically acceptable carrier.

Yet another aspect of the present disclosure provides methods of imaging a tissue in an animal or human host comprising the steps of: administering to an animal or human host a heterodimeric probe according to any of claims 1-30, wherein the probe has a detectable label thereon; detecting the presence of the detectable label in the animal or human host; and identifying a tissue in the animal or human host wherein the amount of the detectable label in the tissue is greater than in other tissues of the host, thereby determining the position of a tissue binding to the heterodimeric probe within the animal or human host.

In embodiments of this aspect of the disclosure, the heterodimeric probe is selected from the group consisting of: formula I, II, IV, VII, VIIIa, and IX, wherein, M+ can be a radionuclide selected from ⁶⁸Ga and ⁶⁴Cu, and as shown in FIGS. 7B, 1B, 11, 13, 22B, and 33 respectively, where M+ may be a radionuclide selected from ⁶⁸Ga and ⁶⁴Cu.

In embodiments of this aspect of the disclosure, the heterodimeric probe may be detected by positron emission tomography or by single photon emission computed tomography.

In embodiments of this aspect of the disclosure, the heterodimeric probe selectively binds to a tumor in the animal or human host, wherein the tumor comprises cells expressing α^{v}β₃ and/or GRPR.

In some embodiments of this aspect of the disclosure, the tumor may be a tumor of the breast, the prostate, a malignant melanoma, an ovarian carcinoma, a gastro-intestinal carcinoma, or a glioblastoma.

Still another aspect of the present disclosure encompasses methods of delivering an agent to a cell, comprising contacting a cell or population of mammalian cells with a heterodimeric probe according to claims 1-30 capable of simultaneously binding to two an integrin and to a gastrin-releasing peptide receptor, and wherein the probe further comprises an agent to be delivered to a target cell or tissue of a mammalian subject; and allowing the heterodimeric probe to bind to an integrin, a gastrin-releasing peptide receptor, or both an integrin and a gastrin-releasing peptide receptor, on the cell or population of mammalian cells, thereby delivering the agent to the cell or cells.

In some embodiments of this aspect of the disclosure, the cell or population of cells comprise mammalian cells, and wherein the cells or population of cells are isolated cells.

In some embodiments of this aspect of the disclosure, the cell or population of cells comprise mammalian cells, and wherein the cells or population of cells are in a tissue of a human or animal host.

In some embodiments of this aspect of the disclosure, the agent is a therapeutic agent or a detectable agent,

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

Now having described the embodiments of the disclosure, in general, the example describes some additional embodiments. While embodiments of present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### EXAMPLES

### Example 1

### Materials and Methods

All chemicals obtained commercially were of analytic grade and used without further purification. ‘No-carrier-added’-¹⁸F-F⁻ was obtained from an in-house PET trace cyclotron (GE Healthcare). Reversed-phase extraction CI8 Sep-Pak cartridges were obtained from Waters and were pretreated with ethanol and water before use. The syringe filter and polyethersulfone membranes (pore size, 0.22 □m; diameter, 13 mm) were obtained from Nalge Nunc International. ¹²⁵I-Echistatin, labeled by the lactoperoxidase method to a specific activity of 74 TBq/mmol (2,000 Ci/mmol) and ¹²⁵I-[Tyr⁴]BBN (74 TBq/mmol (2,000 Ci/mmol)) were purchased from GE Healthcare. Na¹²⁵I was purchased from Perkin-Elmer (Waltham, Mass.). ⁶⁴Cu was obtained from University of Wisconsin (Madison, Wis.).

The peptides Aca-BBN(7-14) and c(RGDyK) (as shown in FIG. 8) were synthesized by Peptides International. RGD-BBN was synthesized as described (Liu et al., (2009) J. Med. Chem. 52: 425-432, incorporated herein by reference in its entirety). 1,4,7,10-tetraazadodecane-N,N′,N″,N′″-tetraacetic acid (DOTA) and S-2-(4-isothiocyanatobenzyl)-1,4,7-triazacyclononane-1,4,7-triacetic acid (p-SCN-Bn-NOTA) were purchased from Macrocyclics (Dallas, Tex.). ⁶⁸Ga was obtained from a ⁶⁸Ge/⁶⁸Ga generator (produced by Cyclotron, Obninsk, Russia) eluted with 4 ml of 0.1 N HCl.

Analytic as well as semi-preparative reversed-phase high-performance liquid chromatography (RP-HPLC) were performed on a Dionex 680 chromatography system with a UVD 170U absorbance detector and model 105S single-channel radiation detector (Carroll & Ramsey Associates). The recorded data were processed using Chromeleon version 6.50 software. Isolation of peptides and ¹⁸F-labeled peptides was performed using a Vydac protein and peptide column (218TP510; 5 μm, 250×10 mm). The flow was set at 5 mL/min using a gradient system starting from 95% solvent A (0.1% trifluoroacetic acid (TFA) in water) and 5% solvent B (0.1% TFA in acetonitrile (ACN)) (0-2 min) and ramped to 35% solvent A, 65% solvent B, at 32 min. The analytic HPLC was performed using the same gradient system, but with a Vydac column (218TP54, 5 fxm, 250×4.6 mm) and a flow of 1 mL/min. The ultraviolet (UV) absorbance was monitored at 218 nm, and the identification of the peptides was confirmed based on the UV spectrum acquired using a photodiode array detector.

The NOTA conjugation and radiolabeling procedures were all performed under metal-free conditions.

### Example 2

### Preparation of NH₂-Glu-BBN(7-14)-c(RGDyK) (BBN-RGD)

The Boc-protected glutamic acid activated ester Boc-E(OSu)₂ was prepared as previously reported by Wu et al., J. Nucl Med. 2005; 46: 1707-1718 incorporated herein by reference in its entirety. To a solution of Boc-E(OSu)₂ (4.4 mg, 10 μmol) in 2 mL anhydrous N,N-dimethylformamide (DMF), 0.8 eq. Aca-BBN(7-14) (8.4 mg, 8 μmol) was added. The pH of the resulting mixture was adjusted to 8.5-9.0 with diisopropylethyl amine (DIPEA). After stirring at room temperature for 2 hr, 1.2 eq. c(RGDyK) (7.6 mg, 12 μmol) was added. The desired product Boc-BBN-RGD was isolated by preparative HPLC. The Boc-group was then removed by anhydrous TFA, and the crude product was again purified by HPLC. A total of 6.9 mg BBN-RGD was obtained as white powder in 48.4% overall yield. Analytic HPLC (retention time [Rₜ]=16.8 min) and mass spectrometry (MALDI-TOF-MS [matrix-assisted laser desorption/ionization time-of-flight mass spectrometry]: m/z 1,783.03 for [MH]⁺ (C₈iH₁₂₃N₂₄O₂₀S, calculated molecular weight [MW] 1,783.90)) confirmed the identity of the purified product.

### Example 3

### Preparation of FB-NH-Glu-BBN(7-14)-c(RGDyK) (FB-BBN-RGD)

N-Succinimidyl-4-fluorobenzoate (SFB) (4 mg, 16.8 Sμmol) and BBN-RGD (2 mg, 1.12 μmol) were mixed in 0.05 M borate buffer (pH 8.5) at room temperature. After 2 hr, the desired product FB-BBN-RGD was isolated by semi-preparative HPLC in 62% yield. Analytic HPLC (Rₜ=18.1 min) and mass spectrometry (MALDI-TOF-MS: m/z 1,905.90 for [MH]⁺ (C₈₈H₁₂₆FN₂₄C₂₁S, calculated [MW] 1,905.92)) analyses confirmed the product identification.

### Example 4

### Radiochemistry

N-Succinimidyl-4-¹⁸F-fluorobenzoate (¹⁸F-SFB) was synthesized according to a previously reported procedure (Wu et al., J. Nuc. Med. (2007) 48: 1536-1544, incorporated herein by reference in its entirety). This procedure has been adapted into a commercially available synthesis module (GE TRACERlab FXFN™). The purified ¹⁸F-SFB was rotary evaporated to dryness, reconstituted in dimethyl sulfoxide (DMSO, 200 μL), and added to a DMSO solution of peptides (0.1 μmol of BBN, RGD, or BBN-RGD) with DIPEA (20 μL).

The peptide mixture was incubated at 60° C. for 30 min. After dilution with 700 μL of 1% TFA, the mixture was purified by semi-preparative HPLC. The desired fractions were combined and rotary evaporated to remove the solvent. The ¹⁸F-labeled peptides were then formulated in normal saline and passed through an 0.22-μm Millipore filter into a sterile multidose vial for in vitro and in vivo experiments.

The synthesis of BBN-RGD heterodimer was performed through an active ester method by coupling Boc-Glu(OSu)₂ with BBN and RGD peptides sequentially. After TFA deprotection, BBN-RGD was obtained as a fluffy white powder with a yield of 48.4%. Four products were generated from the coupling reaction [Boc-Glu(BBN)₂, Boc-Glu(RGD)₂, Boc-Glu-BBN-RGD (RGD on the side-chain 8-position), and Boc-Glu-RGD-BBN (BBN on the side-chain 5-position)]. The Boc-Glu(BBN)₂ and Boc-Glu(RGD)₂ impurities could be efficiently removed. However, there was no observed difference in HPLC retention time between Boc-Glu-BBN-RGD and Boc-Glu-RGD-BBN. Therefore, as shown in FIG. 1B, the final product is a mixture of 2 closely related variant compounds.

The total synthesis time for ¹⁸F-SFB was about 100 min, and the decay-corrected yield was 67%±11% (n=10) using the modified GE synthetic module (TRACERlab FXFN). The decay-corrected radiochemical yield of ¹⁸F-FB-BBN-RGD based on ¹⁸F-SFB was 12.0%±0.7% in=4). The radiochemical purity of ¹⁸F-FB-BBN-RGD was >99% according to analytic HPLC. The specific radioactivities of ¹⁸F-FB-BBN, ¹⁸F-FB-RGD, and ¹⁸F-FB-BBN-RGD were estimated to be about 100 TBq/mmol on the basis of the labeling agent ¹⁸F-SFB, as the unlabeled peptides were efficiently separated from the product. The octanol/water partition coefficient (logP) for ¹⁸F-FB-BBN-RGD was −0.92±0.04 (¹⁸F-FB-RGD, −1.75±0.03; ¹⁸F-FB-BBN, 1.49±0.02), indicating that this tracer is more hydrophilic than ¹⁸F-FB-BBN, but less hydrophilic than ¹⁸F-FB-RGD.

### Example 5

### Synthesis of benzyl 3,3′-(2-(tert-butoxycarbonylamino)ethylazanediyl)dipropanoate

Tert-butyl 2-aminoethylcarbamate (1.6 g, 10 mmol) and benzyl acrylate (16.2 g, 100 mmol) were heated to 70° C. under nitrogen for 7 days. Excess benzyl acrylate was distilled off at 60° C. and the residue was purified by column chromatography to yield 3,3′-(2-(tert-butoxycarbonylamino)ethylazanediyl)dipropanoate as a colorless oil (4.3 g, 89%). ¹H NMR (CDCl₃) δ 7.26-7.36 (m, 10H), 5.11 (s, 1H), 5.04 (bs, 4H), 3.14-3.15 (m, 2H), 2.76 (t, J=6.93 HZ, 4H), 2.51 (t, J=5.73 Hz, 2H), 2.44 (t, J=6.93 Hz, 4H).

### Example 6

### Synthesis of 3,3′-(2-(tert-butoxycarbonylamino)ethylazanediyl)dipropanoic acid

Pd/C (50 mg, 10%) was added to a solution of benzyl 3,3′-(2-(tert-butoxycarbonylamino)ethylazanediyl)dipropanoate (1.5 g, 3.1 mmol) in EtOH (100 ml). The reaction mixture was bubbled with hydrogen overnight. The reaction mixture was filtered through a plug of celite, washed with EtOH (5 mL) and evaporated to dryness to give the crude product as a colorless oil. To the residue was added CH₂Cl₂ (5 mL). The solvent was evaporated to yield 3,3′-(2-(tert-butoxycarbonylamino)ethylazanediyl)dipropanoic acid as a white solid (0.7 g, 74%). ¹H NMR (DMSO) δ 2.93 (m, 2H), 2.65 (t, J=7.1 Hz, 4H), 2.41 (t, J=7.1 Hz, 2H), 2.29 (t, J=9.93 Hz, 4H), 1.38 (s, 9H).

### Example 7

### Synthesis of 3,3′-(2-aminoethylazanediyl)dipropanoic acid (AEADP)-RGD-BBN hetero dimmer (III)

To a solution of 3,3′-(2-(tert-butoxycarbonylamino)ethylazanediyl)dipropanoic acid (3.04 mg, 10 μmol) in DMF (300 μL) was added a solution of TSTU (6.02 mg, 20 μmol) in DMF (600 μL) followed by DIPEA (20 μL). The reaction mixture was stirred for 30 min at room temperature. BBN (10.5 mg, 10 μmol) in DMF (1.0 mL) was added and the reaction mixture was stirred for 20 min at room temperature. After adding RGD (12.4 mg, 20 μmol) in DMF (1.2 mL), the reaction mixture was heated at 60° C. for 1 h. The reaction progress was monitored by analytical HPLC. Once the reaction reached completion, HOAc solution (2 mL, 5%) was added to quench the reaction. The Boc protected crude product was purified by preparative HPLC and lyophilized to yield a white powder. The Boc protection group was removed by dissolve the product in 3 mL TFA and stirred for 10 min at room temperature. After removing excess TFA under reduced pressure, the final product was purified by preparative HPLC and lyophilized to afford AEADP-RGD-BBN hetero dimer as a white powder (5.9 mg, 32% overall yield, two steps). Analytical HPLC (Rₜ=18.1 min) and mass spectrometry (MALDITOF-MS: m/z 1841.76 for [MH]+ (C84H129N25O20S, calculated [MW] 1840.96)) analyses confirmed the product identification.

### Example 8

### Synthesis of FB-AEADP-RGD-BBN

N-Succinimidyl-4-fluorobenzoate (SFB) (2 mg, 8.4 μmol) in DMF (200 μL), AEADP-RGD-BBN (1.8 mg, 1.0 μmol) in DMF (200 μL) and DIPEA (20 μL) were mixed. The reaction mixture was heated at 60° C. for 30 min. The reaction mixture was quenched with 2 mL 5% HOAc. The crude product FB-AEADP-RGD-BBN was purified by preparative HPLC and lyophilized to give a white powder in 85% yield. Analytic HPLC (Rₜ=20.3 min) and mass spectrometry (MALDI-TOF-MS: m/z 1961.95 for [MH]+ (C91H13218FN25O21 S, calculated [MW] 1961.98)) analyses confirmed the product identification.

### Example 9

### Radiochemistry

To a mixture of peptides (200 μg) in DMSO (20 μL) and DIPEA (20 μL) was added ¹⁸F-SFB. The reaction mixture was heated for 15 min at 90° C. The reaction was quenched with 800 μL of 5% HOAc. The ¹⁸F labeled peptide was purified by semi-preparative HPLC. The desired fractions were combined and the solvent was removed under reduced pressure. The 18F-labeled peptide was then formulated in normal saline and passed through an 0.22-μm Millipore filter into a sterile multidose vial for in vitro and in vivo experiments.

### Example 10

### Octanol/Water Partition Coefficient

Approximately 111 kBq of ¹⁸F-FB-BBN, ¹⁸F-FB-RGD, or ¹⁸F-FB-BBN-RGD in 500 μL of PBS (pH 7.4) were added to 500 μL of octanol in an Eppendorf microcentrifuge tube. The mixture was vigorously vortexed for 1 min at room temperature. After centrifugation at 12,500 rpm for 5 min in an Eppendorf microcentrifuge (model 5415R; Brinkman), 200-μL aliquots of both layers were measured using a 7-counter (Packard Instruments). The experiment was performed in triplicate.

### Example 11

### NOTA Conjugation of Peptide

The c(RGDyK) (RGD), Aca-BBN(7-14) (BBN) and RGD-BBN peptides were conjugated with NOTA under standard SCN-amine reaction conditions as previously described (Li et al., (2008) Eur. J. Med. Mol. Imaging. 35: 1100-1108, incorporated herein by reference in its entirety). Briefly, a solution of 2 μmol of peptide (RGD, BBN, or RGD-BBN) was mixed with 6 μmol of p-SCN-Bn-NOTA in sodium bicarbonate buffer (pH 9.0). After stirring at room temperature overnight, the NOTA-conjugated peptides were isolated by semi-preparative HPLC. The desired fractions were combined and lyophilized to afford the final product as a white powder.

NOTA-c(RGDyK) (NOTA-RGD) was obtained in 61% yield with a 13.4 min retention time on analytical HPLC. Matrix-assisted laser desorption/ionization (MALDI) time-of-flight (TOF) mass spectrometry (MS) was m/z 1,070.4 for [MH]+ (C47H68N13O14S, calculated molecular weight 1,070.5 Da). NOTA-BBN was obtained in 72% yield with a 22.05 min retention time on analytical HPLC. MALDITOF-MS was m/z 1504.0 for [MH]+ (C69H102N18O16S2, calculated molecular weight 1503.8).

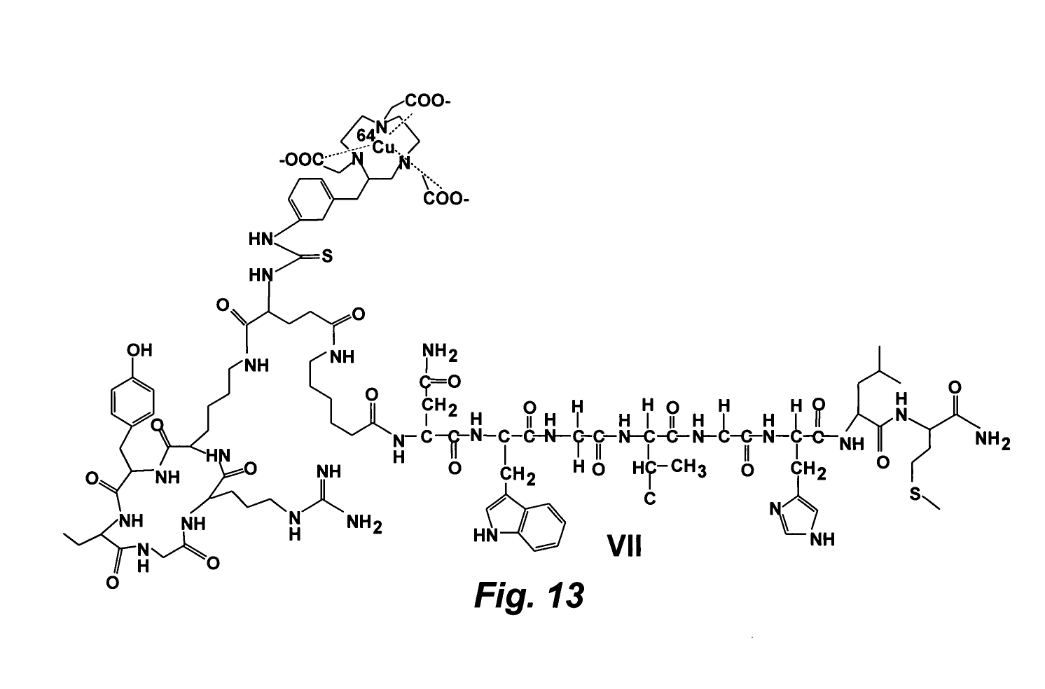

NOTA-RGD-BBN (VII), as shown in FIG. 13, was obtained in 52% yield with a 20.72 min retention time on analytical HPLC. MALDI-TOF-MS was m/z 2235.3 for [MH]+ (C102H149N27O26S2, calculated molecular weight 2234.6 Da).

### DOTA Conjugation of Peptide

RGD-bombesin was also conjugated with DOTA. Briefly, DOTA was activated by 1-ethyl-3-[3-(dimethylamino)-propyl]carbodiimide and N-hydroxysulfonosuccinimide for 30 min with a molar ratio of 10:5:4 for DOTA: 1-ethyl-3-[3-(dimethylamino)-propyl]carbodiimide: N-hydroxysulfonosuccinimide. The DOTA-OSSu (6 mmol, calculated on the basis of N-hydroxysulfonosuccinimide) was added to RGD-bombesin (2 mmol) in 0.1N NaHCO₃ solution (pH 9.0). After being stirred at 4° C. overnight, the DOTA conjugate was isolated by semi-preparative HPLC. DOTA-RGD-bombesin was obtained in 60% yield with more than 95% HPLC purity (Rt, 20.63 min). MALDI-TOF-MS: m/z, 2,171.2 for [MH]1 (C97H148N28O27S, calculated molecular weight, 2,170.4).

On the analytic HPLC, no significant difference in retention time was observed between ⁶⁴Cu-labeled tracers and the unlabeled NOTA and DOTA conjugates. NOTA-RGD-bombesin was more easily labeled with ⁶⁴Cu than was DOTARGD-bombesin as determined by the labeling condition studies, the results of which are shown in FIG. 28A. For in vitro and in vivo studies, the specific activity of the ⁶⁴Cu tracers after labeling and purification was typically about 7.4-14.8 MBq/nmol (0.2-0.4 Ci/mmol), with radiochemical purity greater than 98% as determined by analytic radio-HPLC.

### Example 12

### ⁶⁸Ga Radiolabeling

The ⁶⁸Ga labeling was performed according to methods previously described (Li et al., (2008) Eur. J. Med. Mol. Imaging. 35: 1100-1108, incorporated herein by reference in its entirety). Briefly, 10 nmol of NOTA-RGD, NOTA-BBN, or NOTA-RGD-BBN peptide, was dissolved in 500 μl of 0.1 M sodium acetate buffer and incubated with 185 MBq of ⁶⁸Ga for 10 min at 40° C. ⁶⁸Ga-NOTA-RGD, ⁶⁸Ga-NOTA-BBN, or ⁶⁸Ga-NOTA-RGD-BBN was then purified by analytical HPLC and the radioactive peak containing the desired product was collected. After removing the solvent by rotary evaporation, the activity was reconstituted in PBS and passed through a 0.22-μm Millipore filter into a sterile multidose vial for in vitro and in vivo experiments. The labeling was done with a 92% decay-corrected yield for NOTA-RGD (Rt 12.9 min), 95% for NOTA-BBN (Rt 21.8 min), and 90% for NOTA-RGD-BBN (Rt 19.9 min).

### ⁶⁴Cu Radiolabeling

For in vitro and in vivo studies, 5-10 nmol of NOTA-RGD, NOTA-BBN, NOTA-RGD-BBN, or DOTA-RGD-BBN dissolved in NaOAc buffer was labeled with ⁶⁴Cu in the conditions of 42° C. 1 h for DOTA conjugates, and room temperature 15 min for NOTA-conjugates. The labeled peptides were then purified by analytical HPLC. The radioactive peak containing the desired product was collected and rotary evaporated to remove the solvent. The products were then formulated in phosphate-buffered saline (PBS), and passed through a 0.22-μm Millipore filter into a sterile multidose vial for in vitro and in vivo experiments.

The NOTA conjugates of BBN and RGD-BBN were analyzed by both HPLC and mass spectroscopy to confirm the identity of the products. The characterizations of ¹⁸F-FB-PEG₃-RGD-BBN, ⁶⁴Cu-NOTA-RGD-BBN, and ⁶⁸Ga-NOTA-RGD-BBN are listed in Table 1.

TABLE 1
Characterizations of ¹⁸F-FB-PEG₃-RGD-BBN, ⁶⁴Cu-NOTA-RGD-BBN, and
⁶⁸Ga-NOTA-RGD-BBN.

| | Receptor Affinity IC₅₀ (nM) | | Labeling | Radiochemical | Preparation |
|---|---|---|---|---|---|
| | Integrin αᵥβ₃ | GRPR | Yield | Purity | Time |
| ¹⁸F-FB-PEG₃- | 13.77 ± 1.82 | 73.28 ± 1.57 | 40~50% | >98% | about 180 min |
| RGD-BBN | | | | | |
| ⁶⁴Cu-NOTA- | 16.15 ± 2.77 | 92.75 ± 3.53 | >90% | >98% | about 40 min |
| RGD-BBN | | | | | |
| ⁶⁸Ga-NOTA- | | | >90% | >98% | about 45 min |
| RGD-BBN | | | | | |

Note:
The IC₅₀ was determined by FB-PEG₃-RGD-BBN and NOTA-RGD-BBN. Labeling yield of ¹⁸F-FB-PEG₃-RGD-BBN was based on ¹⁸F-SFB. Preparation time of ¹⁸F-FB-PEG₃-RGD-BBN was determined from ¹⁸F-F.

The decay-corrected labeling yield of ¹⁸F-FB-PEG₃-RGD-BBN was 40-50% based on ¹⁸F-SFB. The decay-corrected labeling yields of ⁶⁴Cu-NOTA-RGD-BBN, and ⁶⁸Ga-NOTA-RGD-BBN were all higher than 90% under the condition of reaction at 40° C. for 15 min. After the purification with HPLC, the radiochemical purity of the tracers were all higher than 98%. The overall preparation time was approximately 180 min for ¹⁸F-FB-PEG₃-RGD-BBN from ¹⁸F-F, approximately 40 min for ⁶⁴Cu-NOTA-RGD-BBN from ⁶⁴CuCl₂, and approximately 45 min for ⁶⁸Ga-NOTA-RGD-BBN from ⁶⁸Ga³⁺ elution.

### Example 13

### Cell Lines and Animal Models

The PC-3 and DU-145 human prostate carcinoma cell lines were purchased from American Type Culture Collection. PC-3 cells were grown in F-12K nutrient mixture (Kaighn's modification) (Invitrogen Corp.), and DU-145 cells were grown in minimum essential medium (Eagle) mixture supplemented with 10% (v/v) fetal bovine serum (Invitrogen) at 37° C. with 5% CO₂. The PC-3 and DU-145 tumor models were generated by subcutaneous injection of 5×10⁶ tumor cells into the front flank of male athymic nude mice (Harlan). The mice were subjected to micro-PET studies when the tumor volume reached 100-300 mm³ (3-4 wk after inoculation).

The PC-3 human prostate carcinoma cell line and MDAMB-435 human melanoma cell line (Lacroix M. (2009) Cancer Chemother. Pharmacol. 63: 567; Rae et al., (2007) Breast Cancer Res. Treat.; 104:1) were purchased from the American Type Culture Collection (ATCC, Manassas, Va.).

The 4T1 murine breast cancer cell line was purchased from American Type Culture Collection and grown in RMPI 1640 medium (Invitrogen Corp.) supplemented with 10% (v/v) fetal bovine serum (Invitrogen) at 37° C. with 5% CO₂. The 4T1 tumor model was generated by subcutaneous injection of 5×10⁶ tumor cells into the left front flank of female normal BALB/c mice (Harlan). The mice were used for micro-PET studies when the tumor volume reached 100˜300 mm³ (about 1-2 weeks for 4T1 tumor model).

The MDA-MB-231, MDA-MB468, BT474, BT-20, T47D, MCF-7 and MDA-MB435 human breast cancer cell lines were all obtained from the American Type Culture Collection (ATCC) and maintained under standard conditions according to ATCC. The MDA-MB435 tumor model was established by orthotopic injections of 5×10⁶ cells into the right mammary fat pad of female athymic nude mice. For T47D tumor model establishment, the female nude mice were first subcutaneously implanted with 60-day release 17β-estradiol pellets (Innovative Research of America, Sarasota, Fla.) in the left neck. One day after the estradiol implantation, 1×10⁷ T47D cells were orthotopically injected into the right mammary fat pad of the nude mice. The mice were subjected to microPET studies when the tumor volume reached 100-300 mm³ (2-3 weeks for MDA-MB-435, and 4-5 wk for T47D).

### Example 14

### In Vitro Cell-Binding Assay

In vitro integrin αᵥβ₃-binding affinities and specificities of RGD, BBN-RGD, and FB-BBN-RGD were assessed via displacement cell-binding assays using ¹²⁵¹-echistatin as the radioligand. Experiments were performed on U87MG human glioblastoma cells by a previously described method (Wu et al., J. Nucl. Med. (2005) 46: 1707-1718, incorporated herein by reference in its entirety). In vitro GRPR-binding affinities and specificities of BBN, BBN-RGD, and FB-BBN-RGD were assessed via displacement cell-binding assays using ¹²⁵I-[Tyr⁴]BBN as the radioligand. Experiments were performed on PC-3 human prostate carcinoma cells by a previously described method (Chen et al., J. Nucl. Med. (2004) 45: 1390-1397, incorporated herein by reference in its entirety). The best-fit 50% inhibitory concentration (IC₅₀) values were calculated by fitting the data with nonlinear regression using Graph-Pad Prism (GraphPad Software, Inc.). Experiments were performed with triplicate samples.

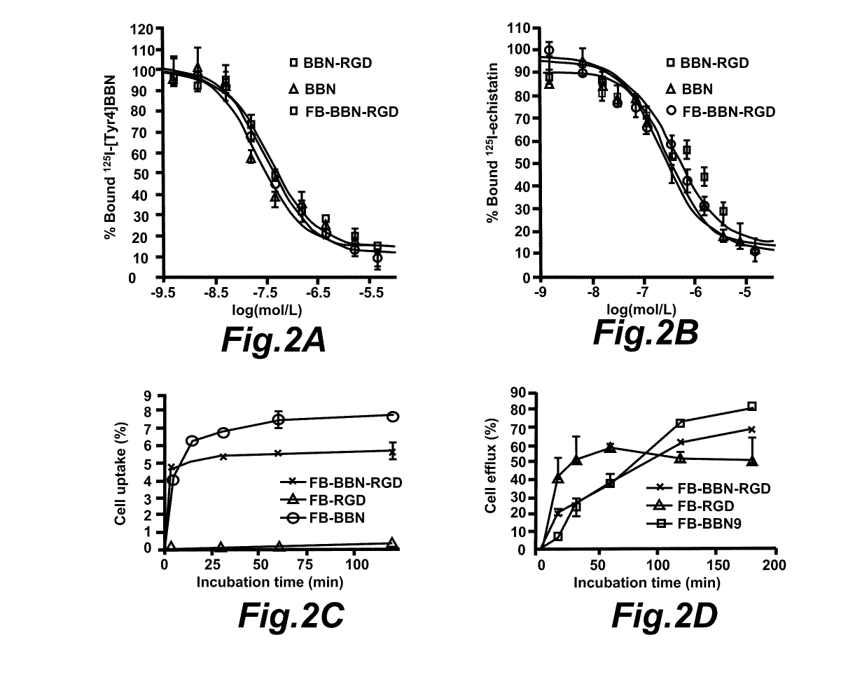

The binding affinities of Aca-BBN(7-14), BBN-RGD, and FB-BBN-RGD for GRPR were evaluated for PC-3 cells. Results of the cell-binding assay were plotted in sigmoid curves for the displacement of ¹²⁵¹-[Tyr⁴]BBN from PC-3 cells as a function of increasing concentration of BBN analogs. The IC₅₀ values were determined to be 20.7±3.2 nM for BBN monomer, 35.7±4.4 nM for heterodimer BBN-RGD, and 32.0±1.9 nM for FB-BBN-RGD on 10⁵ PC-3 cells, as shown in FIG. 2A. The integrin αᵥβ₃ receptor-binding affinity of RGD, BBN-RGD, and FB-BBN-RGD was determined by performing competitive displacement studies with ¹²⁵I-echistatin. All peptides inhibited the binding of ¹²⁵I-echistatin (integrin αᵥβ₃-specific) to U87MG cells in a concentration-dependent manner. The IC₅₀ values for RGD, BBN-RGD, and FB-BBN-RGD were 202±28, 428±57, and 282±34 nM, respectively (n=3) (FIG. 2B). The comparable IC₅₀ values from these 2 sets of experiments suggest that the BBN-RGD peptide possesses comparable GRPR and integrin αᵥβ₃ receptor-binding affinities, comparable to those of the corresponding monomer.

In vitro integrin αᵥβ₃-binding affinities and specificities of RGD-BBN, NOTA-RGD-BBN, and BBN were compared with those of RGD via displacement cell-binding assays using ¹²⁵I-c[RGDyK] as the radioligand. ¹²⁵I-c(RGDyK) was prepared by labeling c(RGDyK) with Na¹²⁵I at high specific activity (about 44.4 TBq/mmol) (Chen et al., Mol Imaging Biol. (2004) 6: 350-359, incorporated herein by reference in its entirety).

Experiments were performed on U87MG human glioma cells expressing integrin αᵥβ₃ as previously described (Wu et al., (2007) Eur. J. Nucl. Med. Mol. Imaging. 34: 1823-1831; Cai et al., (2006) J. Nuc. Med. 47: 1172-1180, incorporated herein by reference in their entireties). In vitro GRPR binding affinities and specificities of RGD-BBN, NOTA-RGD-BBN and RGD were compared with those of BBN via displacement cell-binding assays using ¹²⁵I-[Tyr4] BBN as the radioligand. Experiments were performed on GRPR-expressing PC-3 cells following our previously described procedure (Chen et al., Mol Imaging Biol. (2004) δ: 350-359; Cai et al., (2006) J. Nuc. Med. 47: 1172-1180, incorporated herein by reference in their entireties). The best-fit 50% inhibitory concentration (IC₅₀) values were calculated by fitting the data with nonlinear regression using GraphPad Prism (GraphPad Software). Experiments were performed twice with triplicate samples.

The integrin αᵥβ₃ receptor-binding affinities of RGD-BBN, NOTA-RGD-BBN were determined by performing competitive binding assay with ¹²⁵I-c(RGDyK) as the radioligand. Cyclic RGD peptide c(RGDyK) and BBN were also added for comparison. RGD-BBN and NOTA-RGD-BBN inhibited the binding of ¹²⁵I-c(RGDyK) to integrin-expressing U87MG cells in a concentration-dependent manner. The IC₅₀ values for RGD-BBN, NOTA-RGD-BBN and c(RGDyK) were 17.91±5.70 nM, 22.57±6.68 nM, and 11.19±4.21 nM, respectively (FIG. 14A). BBN did not show significant binding inhibition of ¹²⁵I-c(RGDyK). The binding affinities of RGD-BBN, NOTA-RGD-BBN, and Aca-BBN(7-14) for GRPR were evaluated using GRPR-positive PC-3 cells with ¹²⁵I-[Tyr⁴]BBN as the radioligand.

The results of the cell-binding assay were plotted as sigmoid curves for the displacement of ¹²⁵I-[Tyr⁴]BBN from PC-3 cells as a function of increasing concentration of BBN analogs. The IC₅₀ values were determined to be 67.92±4.97 nM for RGD-BBN, 55.89±4.23 nM for NOTA-RGD-BBN, and 78.96±4.86 nM for BBN on PC-3 cells. RGD did not show significant binding inhibition of ¹²⁵I-[Tyr⁴]BBN with GRPR (FIG. 14B). The IC₅₀ values from these two sets of experiments suggest that RGD-BBN possessed comparable GRPR and integrin αᵥβ₃ receptor-binding affinities with the corresponding unmodified monomers. Conjugation of NOTA had little effect on the integrin and GRPR receptor-binding characteristics.

The integrin αᵥβ₃ receptor-binding affinities of DOTA-RGD-bombesin and NOTA-RGD-bombesin were compared with c(RGDyK) by conducting a competitive binding assay on U87MG cells using 125I-c(RGDyK) as the radioligand. The inhibitory concentrations of 50% for DOTA-RGD-BBN, NOTA-RGD-BBN, and c(RGDyK) were 21.55 6 2.19 nM, 16.15 6 2.77 nM, and 10.84 6 2.55 nM, respectively, as shown in FIG. 28B. The binding affinities of DOTA-RGD-BBN, NOTA-RGD-BBN, and Aca-BBN(7-14) for GRPR were evaluated on GRPR-positive PC-3 cells using 125I-[Tyr4]BBN as the radioligand. The inhibitory concentrations of 50% were determined to be 85.79 6 2.08 nM for DOTA-RGD-BBN, 92.75 6 3.53 nM for NOTA-RGD-BBN, and 71.57 6 3.06 nM for Aca-BBN(7-14) on PC-3 cells, as shown in FIG. 28C.

GRPR and Integrin αᵥβ₃ Expression on Breast Cancer Cells

The expression of GRPR and integrin αᵥβ₃ on various breast cancer cells were determined by radioligand receptor-binding assay using ¹²⁵I-[Tyr4]BBN and ¹²⁵I-c(RGDyK), respectively. As shown in FIG. 40A, among all the breast cancer cells tested, only two cell lines (T47D and BT474) expressed GRPR. T47D expressed the highest level of GRPR as the cell binding percent of ¹²⁵I-[Tyr4]BBN was the highest. The binding specificity of ¹²⁵I-[Tyr4]BBN with T47D cells was confirmed by blocking study with cold BBN. The expression of integrin αᵥβ₃ on the breast cancer cell lines followed the order of MDA-MB-435>MDA-MB-231>MDA-MB-468>T47D>MCF-7>BT20, while BT474 cells expressed undetectable integrin αᵥβ₃ (FIG. 40B). After blocking with cold c(RGDyK), the cell bound ¹²⁵I-c(RGDyK) all decreased to a background level, indicating the binding of ¹²⁵I-c(RGDyK) with the tumor cells was integrin αᵥβ₃-mediated specific binding.

### Example 15

Cell Uptake and Efflux Studies

(i) Uptake and Efflux of ¹⁸F-FB-BBN, ¹⁸F-FB-RGD, and ¹⁸F-FB-BBN-RGD into PC-3 Cells

Uptake and efflux of ¹⁸F-FB-BBN, ¹⁸F-FB-RGD, and ¹⁸F-FB-BBN-RGD into PC-3 cells were examined according to the following protocol. In the cell uptake experiment, PC-3 cells were seeded into 12-well plates at a density of 5×10⁵ cells per well for overnight incubation. Cells were rinsed 3 times with phosphate-buffered saline (PBS), followed by the addition of ¹⁸F-FB-RGD, ¹⁸F-FB-BBN, or ¹⁸F-BBN-RGD to the cultured wells in triplicate (about 2 μCi/well). After incubation at 37° C. for 5, 15, 30, 60, and 120 min, cells were rinsed 3 times with PBS and lysed with NaOH-sodium dodecyl sulfate (SDS) (0.2 M NaOH, 1% SDS). The cell lysate was collected in measurement tubes for counting. The cell uptake was normalized in terms of added radioactivity.

In the cell efflux experiment, PC-3 cells were seeded into 12-well plates at a density of 5×10⁵ cells per well for overnight incubation. Cells were rinsed 3 times with PBS and then the appropriate ¹⁸F-labeled peptide tracer was added. The cells were incubated at 37° C. for 2 hr, washed with PBS, and then reincubated with serum-free medium. The cells were washed at different time points (0, 15, 30, 60, 120, 180 min) with PBS and lysed with NaOH-SDS (0.2 M NaOH, 1% SDS). The cell lysate was collected in measurement tubes for counting. Efflux values at different time points were calculated by subtracting retention from 0-min retention, and normalized by dividing the total counts at 0 min.

Due to the relative low receptor-binding affinity of the monomeric RGD peptides and moderate integrin receptor density of the PC-3 cells, ¹⁸F-FB-RGD had relatively low cell uptake (<0.5%). On the other hand, PC-3 cells express a high level of GRPR, ¹⁸F-FB-BBN binding to GRPR facilitates effective internalization of this radioligand, and the uptake of ¹⁸F-FB-BBN is thus rapid and high, reaching about 7% within 30 min of incubation, and plateaus afterward. The cell uptake behavior of ¹⁸F-FB-BBN-RGD is similar to that of ¹⁸F-FB-BBN but the uptake value is slightly lower (FIG. 2C). All three tracers showed substantial efflux when the labeled cells were cultured in serum-free medium devoid of radioactivity (FIG. 2D). The cell uptake protocol used in this study did not distinguish between cell-surface bound and internalized activity.

The cell uptake studies were performed as we have previously described with some modifications (Chen et al., Mol Imaging Biol. (2004) δ: 350-359; Cai et al., (2006) J. Nuc. Med. 47: 1172-1180, incorporated herein by reference in their entireties). Briefly, PC-3 cells were seeded into 12-well plates at a density of 5×10⁵ cells per well and incubated (about 18 kBq/well) with ⁶⁸Ga-labeled tracers at 37° C. for 15, 30, 60, and 120 min. Tumor cells were then washed three times with chilled PBS and harvested by trypsinization with 0.25% trypsin/0.02% EDTA (Invitrogen). The cell suspensions were collected and measured in a y counter (Packard, Meriden, Conn.). The cell uptake was expressed as the percent added dose (% AD) after decay correction. Experiments were performed twice with triplicate wells. For efflux studies, ⁶⁸Ga-labeled tracers (about 18 kBq/well) were first incubated with PC-3 cells in 12-well plates for 1 h at 37° C. to allow internalization. Then cells were washed twice with PBS, and incubated with cell culture medium for 15, 30, and 60 min. After washing three times with PBS, cells were harvested by trypsinization with 0.25% trypsin/0.02% EDTA. The cell suspensions were collected and measured in a γ counter. Experiments were performed twice with triplicate wells. Data are expressed as percent added dose after decay correction.

### (ii) Uptake and Efflux of ⁶⁸Ga-NOTA-RGD-BBN Evaluated in PC-3

The cell uptake and efflux of ⁶⁸Ga-NOTA-RGD-BBN were evaluated in studies in PC-3 tumor cells that express high levels of GRPR and moderate levels of integrin αᵥβ₃ (2.7×10⁶ GRPRs per cell and 2.76×10³ integrins per cell, as described by Zhang et al., (2006) J. Nucl. Med.; 47: 113-121, and Cai et al., (2006) Cancer Res. 66: 9673-9681).

⁶⁸Ga-NOTA-BBN had rapid and high cell uptake (FIG. 14C), which is similar to those of ⁶⁴Cu and ¹⁸F labeled BBN tracers that have been previously reported (Zhang et al., (2006) J. Nucl. Med. 47: 492-501; Chen et al., (2004) J. Nucl. Med. 45: 1390-1397). ⁶8Ga-NOTA-RGD had relatively low cell uptake, which may be due to the low integrin receptor density of PC-3 cells. The cell uptake curve of ⁶⁸Ga-NOTA-RGDBBN was between those of ⁶⁸Ga-NOTA-BBN and ⁶⁸Ga-NOTA-RGD. ⁶⁸Ga-NOTA-RGD-BBN uptake reached a plateau at 15 min incubation (1.69±0.29% AD) and remained at a similar level for up to 2 h. However, the cell uptake protocol used did not distinguish between cell surface-bound and internalized activity. The cell retention of the three tracers also showed the order BBN>RGD-BBN>RGD. The cell-associated RGD peptide was undetectable after 15 min. The cell retention of both ⁶⁶Ga-NOTA-BBN and ⁶⁸Ga-NOTA-RGD-BBN decreased with time. At 60 min, the cell-associated tracers were 6.70±1.00% AD for BBN, 1.14±0.50% AD for RGD-BBN, and 0.10±0.13% AD for RGD, respectively, as shown in FIG. 14D.

(iii) Uptake and Efflux of ⁶⁴Cu-NOTA-RGD-BBN and ⁶⁴Cu-DOTA-RGD-BBN

⁶⁴Cu-NOTA-BBN showed a rapid and high uptake in the PC-3 tumor cells, whereas ⁶⁴Cu-NOTA-RGD had low cell uptake, as shown in FIG. 23A. The cell uptake values of ⁶⁴Cu-NOTA-RGD-BBN and ⁶⁴Cu-DOTA-RGD-BBN were between those of ⁶⁴Cu-NOTA-BBN and ⁶⁴Cu-NOTA-RGD. The amplified cell uptake comparison of ⁶⁴Cu-NOTA-RGD-BBN and ⁶⁴Cu-DOTA-RGD-BBN is shown in FIG. 28B. Cell uptake of ⁶⁴Cu-NOTA-RGD-BBN was slightly more rapid than that of ⁶⁴Cu-DOTA-RGD-BBN during the first 30 min of incubation and then reached a similar value at 60 min. At 120 min, the cell uptake value was 3.70% 6 0.02% added dose for ⁶⁴Cu-DOTA-RGD-BBN and 2.94% 6 0.51% added dose for ⁶⁴Cu-NOTA-RGD-BBN. The cell efflux ratio of ⁶⁴Cu-NOTA-RGD was higher than that of ⁶⁴Cu-NOTA-BBN or ⁶⁴Cu-NOTA-RGD-BBN (FIG. 2B). Because ⁶⁴Cu-NOTA-RGD did not seem to be internalized into the PC-3 cells, the cell efflux reflected mainly dissociation of ⁶⁴Cu-NOTA-RGD from the tumor cells, as the RGD monomer has relatively low affinity for integrin αᵥβ₃. Compared with ⁶⁴Cu-NOTA-BBN, ⁶⁴Cu-NOTA-RGD-BBN showed a relatively low efflux ratio with time. After 2 h, the efflux of both ⁶⁴Cu-NOTA-RGD-BBN and ⁶⁴Cu-NOTA-BBN reached a plateau, indicating that the cells maintained similar activity from a 2 to 10-h period. Although the efflux ratio was much lower for ⁶⁴Cu-NOTA-RGD-BBN than for ⁶⁴Cu-NOTA-BBN, cell retention was much higher for ⁶⁴Cu-NOTA-BBN than for 4Cu-NOTA-RGD-BBN for up to 10 h (FIG. 28C).

(iv) T47D and MDA-MB435 tumor cells were seeded into 12-well plates at a density of 5×10⁵ cells per well one day before experiment to allow adherence. Cells were incubated with ¹⁸F-FB-PEG₃-RGD-BBN, ⁶⁴Cu-NOTA-RGD-BBN or ⁶⁸Ga-NOTA-RGD-BBN (approximately 18 kBq/well) at 37° C. for 15, 30, 60, and 120 min. Tumor cells were then washed three times with chilled PBS and harvested by trypsinization with 0.25% trypsin/0.02% EDTA (Invitrogen, Carlsbad, Calif.). The cells suspensions were collected and measured in a y counter (Packard, Meriden, Conn.). The cell uptake was expressed as the percent added dose (% AD) after decay correction. Experiments were performed twice with triplicate wells.

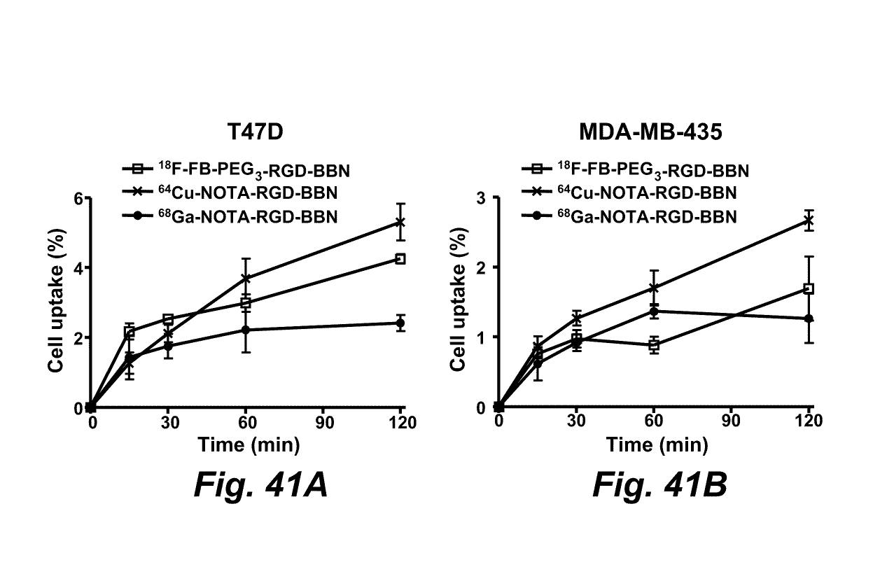

The cell uptake studies of ¹⁸F-FB-PEG₃-RGD-BBN, ⁶⁴Cu-NOTA-RGD-BBN and ⁶⁸Ga-NOTA-RGD-BBN were performed on T47D and MDA-MB-435 tumor cells. As shown in FIGS. 41A and 41B, all the tracers exhibited an increasing uptake with time on both the tumor cells. Generally, the cell uptake levels of the three tracers on the T47D cells were all higher than those on the MDA-MB-435 tumor cells, which may due to the higher GRPR expression of the T47D cells, and the GRPR is more easily to be internalized into the cells than integrin. On both tumor cells, the uptake of ⁶⁴Cu-NOTA-RGD-BBN was significantly higher than that of ¹⁸F-FB-PEG₃-RGD-BBN and ⁶⁸Ga-NOTA-RGD-BBN at the late time points (P<0.05). For example, the T47D cell uptake value at 120 min was 4.25±0.13% AD for ¹⁸F-FB-PEG₃-RGD-BBN, 5.30±0.53% AD for ⁶⁴Cu-NOTA-RGD-BBN, and 2.42±0.23% AD for ⁶⁸Ga-NOTA-RGD-BBN, respectively (n=3).

### Example 16

### Small-Animal PET Studies

(a) PET scans and image analysis were performed using a micro-PET R4 rodent model scanner (Siemens Medical Solutions) as reported by Li et al., J. Nucl. Med. (2007) 48: 1162-1171 and Wu et al., J Nucl. Med. (2005) 46: 1707-1718, both of which are incorporated herein by reference in their entireties). Tumor-bearing mice were each tail-vein injected with approximately 3.7 MBq (100 μCi) of ¹⁸F-FB-RGD, ¹⁸F-FB-BBN, or ¹⁸F-FB-BBN-RGD under isoflurane anesthesia. Five-minute static PET images were then acquired at 0.5, 1, and 2 h after injection. The images were reconstructed by a 2-dimensional ordered-subsets expectation maximum (OSEM) algorithm. No attenuation or scatter correction was applied. For the receptor-blocking experiment, c(RGDyK) (10 mg/kg), Aca-BBN(7-14) (15 mg/kg), or RGD+BBN (10 mg/kg RGD and 15 mg/kg BBN) were co-injected with 3.7 MBq of ¹⁸F-FB-BBN-RGD to PC-3 tumor mice. The 5-min static PET scans were then acquired at 1 hr after injection. For each small-animal PET scan, regions of interest (ROIs) were drawn over the tumor, normal tissue, and major organs by using vendor software (ASI Pro 5.2.4.0) on decay-corrected whole-body coronal images. The average radioactivity concentration (accumulation) within a tumor or an organ was obtained from mean pixel values within the multiple ROI volume, which were converted to counts/mL/min using a conversion factor. Assuming a tissue density of 1 g/mL, the ROIs were converted to counts/g/min, and then divided by the administered activity to obtain an imaging ROI-derived percentage injected dose per gram of tissue (% ID/g).

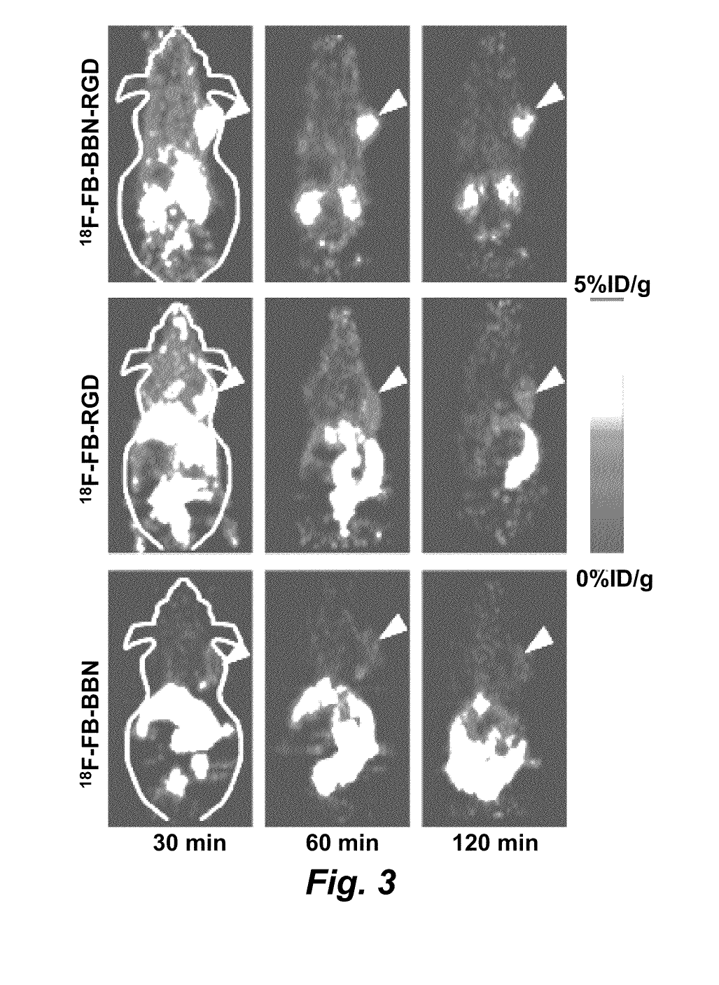

Static small-animal PET scans were performed on a PC-3 xenograft model (n=3, both GRPR- and integrin αᵥβ₃-positive) (Cooper et al., Neoplasia. (2002) 4: 191-194; Zheng et al., J. Biol. Chem. (2000) 275: 24565-24574; Cai et al., Cancer Res. (2006) 66: 9673-9681; Markwalder & Reubi, Cancer Res. (1999) 59: 1152-1159 incorporated herein be reference in their entireties), and selected coronal images at different time points after injection of ¹⁸F-FB-BBN-RGD, ¹⁸F-FB-BBN, or ¹⁸F-FB-RGD are shown in FIG. 3. The tumor was clearly visible with high contrast to contra-lateral background for ¹⁸F-FB-BBN-RGD. Quantitation of tumor and major organ activity accumulation in small-animal PET scans was realized by measuring ROIs encompassing the entire organ on the coronal images. The averaged time-activity curves of these three tracers for the PC-3 tumor, liver, kidneys, and muscle are shown in FIGS. 4A-4E. ¹⁸F-FB-BBN and ¹⁸F-FB-RGD indicated moderate initial tumor uptake in this dual-receptor-positive tumor model; however, the unfavorable hepatobiliary excretion (high liver, bile, and intestinal activity accumulation) limited further applications of these 2 radiotracers, especially in an attempt to detect lesions in the lower abdomen. The PC-3 tumor uptake of ¹⁸F-FB-BBN-RGD was calculated to be 5.00±0.28, 3.57±0.27, and 2.79±0.28% ID/g at 30, 60, and 120 min after injection, significantly higher than those for ¹⁸F-FB-BBN (0.63±0.16, 0.44±0.10, and 0.29±0.08% ID/g at 30, 60, and 120 min after injection, P<0.001), ¹⁸F-FB-RGD (2.21±0.32, 1.30±0.10, and 0.89±0.08% ID/g at 30, 60, and 120 min after injection P<0.01), and even the sum of the uptake for these two monomeric tracers (dotted line in FIG. 4A; P<0.01).

¹⁸F-FB-BBN-RGD also showed substantially lower liver and significantly decreased renal uptake compared with ¹⁸F-FB-BBN (slightly increased kidney uptake compared with ¹⁸F-FB-RGD). Because of the enhanced tumor-targeting efficacy and improved in vivo pharmacokinetics, ¹⁸F-FB-BBN-RGD had higher tumor-to-organ ratios than ¹⁸F-FB-BBN and ¹⁸F-FB-RGD, as shown in FIG. 4E. We also tested this tracer in an integrin αᵥβ₃-positive but low GRPR-expressing DU-145 tumor model. No significant difference in normal organs and tissues was found between these tumor models. Because of the low GRPR expression, ¹⁸F-FB-BBN was unable to detect the DU-145 tumor. However, both ¹⁸F-FB-BBN-RGD and ¹⁸F-FB-RGD were able to visualize this tumor model since it is integrin αᵥβ₃-positive. The DU-145 tumor uptake for ¹⁸F-FB-BBN-RGD was calculated to be 2.18±0.24, 1.49±0.17, and 1.02±0.23% ID/g at 30, 60, and 120 min after injection.

The receptor specificity of ¹⁸F-FB-BBN-RGD in vivo was confirmed by several blocking experiments, as shown in FIGS. 5A and 5B. Representative coronal images of PC-3 tumor mice after injection of ¹⁸F-FB-BBN-RGD in the presence of c(RGDyK) (10 mg/kg), Aca-BBN(7-14) peptide (15 mg/kg), or RGD+BBN (10 mg/kg for RGD and 15 mg/kg for BBN) are illustrated in FIG. 5A. Uptake in the tumor at 1 hr after injection (3.57±0.27% ID/g) was inhibited only partially by either RGD (1.80±0.19% ID/g) or BBN peptide alone (1.35±0.26% ID/g). When both RGD and BBN were co-administered with ¹⁸F-FB-BBN-RGD, the tumor uptake was reduced further to the background level (0.87±0.27% ID/g at 1 hr after injection).

(b) Each PC-3 or MDA-MB-435 tumor-bearing mouse was injected in a tail vein with about 3.7 MBq (100 μCi) of ⁶⁸Ga-NOTA-RGD, ⁶⁸Ga-NOTA-BBN or ⁶⁸Ga-NOTA-RGD-BBN under isoflurane anesthesia (n=4 per group). For static PET, 5-min scans were acquired at 30 min, 1 h, and 2 h after injection. For dynamic PET, 30-min scans (1×30 s, 4×1 min, 1×1.5 min, 4×2 min, 5×3 min; total 15 frames) were started 1 min after injection, and two 5-min static PET images were also acquired at 1 h and 2 h after injection. The images were reconstructed using a two-dimensional ordered subsets expectation maximum (OSEM) algorithm and no correction was applied for attenuation or scatter. For the blocking experiment, PC-3 tumor-bearing mice were co-injected with c(RGDyK) (RGD) at 10 mg/kg body weight, Aca-BBN (7-14) (BBN) at 15 mg/kg or RGD at 10 mg/kg+BBN at 15 mg/kg and 3.7 MBq of ⁶⁸Ga-NOTA-RGD-BBN, and 5-min static PET scans were then acquired at 1 h after injection (n=3 per group). For each small-animal PET scan, regions of interest (ROIs) were drawn over the tumor, normal tissue, and major organs using vendor software ASI Pro 5.2.4.0 on decay-corrected whole-body coronal images. The maximum radioactivity concentrations (accumulation) within a tumor or an organ were obtained from mean pixel values within the multiple ROI volume, and were converted to megabecquerels per milliliter per minute using a conversion factor. These values were then divided by the administered activity to obtain (assuming a tissue density of 1 g/ml) an image ROI-derived percent injected dose per gram (% ID/g).

Representative coronal small-animal PET images of PC-3 tumor-bearing mice (n=4 per group) at different times after intravenous injection of 3.7 MBq (100 μCi) of ⁶⁸Ga-NOTA-RGD-BBN are shown in FIG. 15. ⁶⁸Ga-NOTA-BBN and ⁶⁸Ga-NOTA-RGD were used as controls. The tumors after injection of ⁶⁸Ga-NOTA-RGD-BBN and ⁶⁸Ga-NOTA-BBN were clearly visible with high contrast in relation to the contra-lateral background at all time points measured from 30 to 120 min. ⁶⁸Ga-NOTA-RGD showed low uptake in PC-3 tumors due to the relatively low expression of integrin αᵥβ₃ of PC-3 cells and low affinity of monomer RGD peptide with integrin. Prominent uptake of ⁶⁸Ga-NOTA-RGD-BBN was also observed in the kidneys at early time points, indicating that this tracer is mainly excreted through the renal-urinary route.

Tumor and major organ activity accumulation in the small-animal PET scans was quantified by measuring the ROIs that encompassed the entire organ on the coronal images. The tumor uptake of ⁶⁸Ga-NOTA-RGD-BBN was determined to be 6.55±0.83, 5.26±0.32, and 4.04±0.28% ID/g at 30, 60, and 120 min (FIG. 16A). The liver uptake was very low, the highest being about 2% ID/g at 30 min after injection (FIG. 16B). A comparison of the kinetics of ⁶⁸Ga-labeled RGD-BBN, BBN and RGD tracers in the tumor, liver and kidneys is shown in FIG. 16A-16D. The tumor uptake of ⁶⁸Ga-labeled RGD-BBN was significantly higher than that of BBN and RGD tracers at all time points examined (p<0.05), except that there was no significant difference at 30 min after injection compared with BBN. The tumor uptake of BBN tracer was also higher than that of RGD at all time points, which was mainly due to a high expression of GRPR and low expression of integrin in the PC-3 tumors. The liver uptakes of the three tracers were all very low, with the highest being 2% ID/g for BBN at 30 min after injection. The liver uptake of RGD was somewhat lower than that of BBN and RGD-BBN.

The kidney uptake of ⁶⁸Ga-NOTA-RGD-BBN and ⁶⁸Ga-NOTA-BBN was significantly higher than that of RGD at all time points (p<0.05), as shown in FIG. 16C. The kidney uptake of RGD-BBN was slightly higher than that of BBN at all time points. Due to the rapid clearance of the tracers, the T/NT ratios increased with time for all three tracers.

The tumor/kidney, tumor/liver and tumor/muscle ratios of the three tracers are shown for 1 h after injection in FIG. 16D. The tumor/kidney and tumor/liver ratios of RGD-BBN were significantly higher than those of BBN and RGD (p<0.01). The tumor/muscle ratios followed the order BBN>RGD-BBN>RGD (n=4 per group).

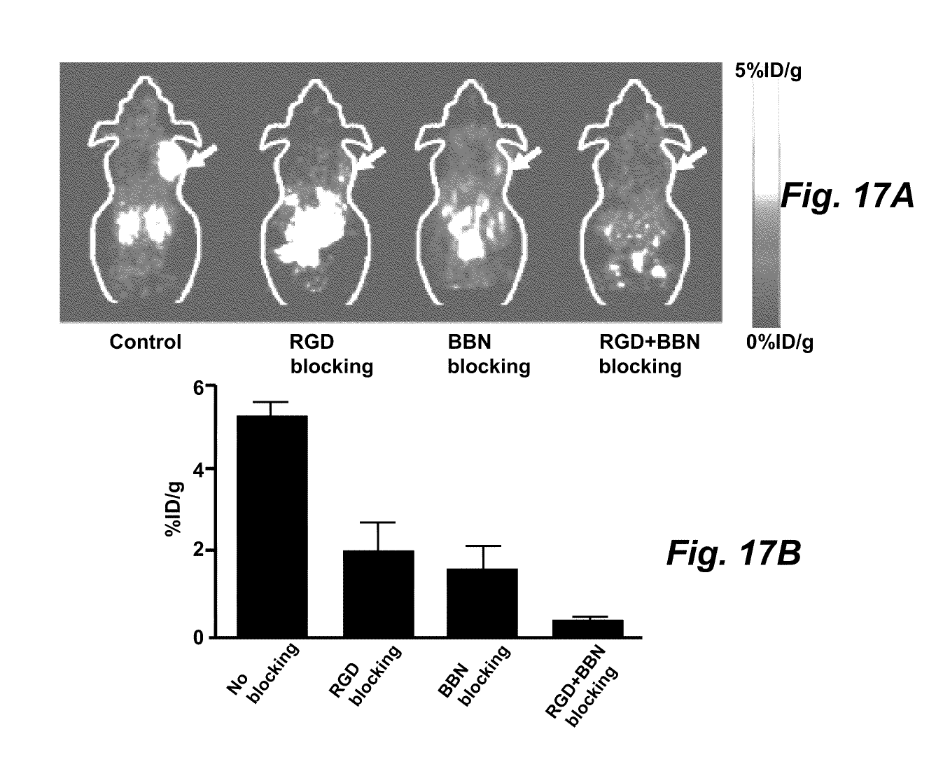

The in vivo integrin and GRPR dual receptor binding property of ⁶⁸Ga-NOTA-RGD-BBN was confirmed by several blocking studies, as shown in (FIGS. 17A and 17B). Representative coronal images of PC-3 tumor mice at 1 h after injection of ⁶⁸Ga-NOTA-RGD-BBN in the presence of RGD (10 mg/kg), BBN (15 mg/kg), or both RGD and BBN (10 mg/kg of RGD and 15 mg/kg of BBN) are shown in FIG. 17A. The tumor uptake of ⁶⁸Ga-NOTA-RGD-BBN was partially inhibited by RGD (from 5.26±0.32% ID/g to 2.08±0.65% ID/g), and by BBN (from 5.26±0.32% ID/g to 1.61±0.57% ID/g). However, when ⁶⁸Ga-NOTA-RGD-BBN was co-administered with RGD and BBN, the tumor uptake was significantly inhibited to the background level (0.42±0.10% ID/g, as shown in FIG. 17B, n=3 per group).

The in vivo behaviors of ⁶⁸Ga-NOTA-RGD-BBN, ⁶⁸Ga-NOTA-BBN, and ⁶⁸Ga-NOTA-RGD were also tested in a MDA-MB435 tumor model, which expresses moderate levels of integrin αᵥβ₃, but undetectable levels of GRPR (based on radioligand binding assays). As shown in FIG. 18A, the BBN tracer did not show significant uptake in the MDA-MB435 tumors, while RGD tracer showed good tumor contrast at 1 h after injection. ⁶⁸Ga-NOTA-RGD-BBN also showed clear tumor uptake due to the integrin recognition of RGD-BBN in vivo. At 60 min after injection, the tumor uptake values of RGD-BBN, BBN, and RGD were 3.23±0.86, 0.38±0.50 and 1.63±0.59% ID/g, respectively, as shown in FIG. 18B.

The tumor targeting property of ⁶⁸Ga-NOTA-RGD-BBN in PC-3 tumor-bearing mice was also evaluated by a 30-min dynamic small-animal PET scan followed by 5-min static scans at 1 h and 2 h after injection. Representative coronal images and quantified % ID/g by ROI analysis at different time points after injection are shown in FIGS. 19A and 19B. High tumor uptake was observed as early as 5 min after injection. The PC-3 tumor uptake was 5.56, 6.68, 6.70, 4.47 and 3.75% ID/g at 5, 15, 30, 60 and 120 min after injection, respectively. With clearance of the tracer from the blood and normal organs, the tumor contrast increased with time. The tracer was excreted mainly through the kidneys, as evidenced by the higher renal uptake at early time points and excretion via the bladder. Kidney uptake reached a peak at about 10 min after injection and then decreased with time. At 120 min after injection, the tumor uptake of the tracer was higher than that in any other normal organ (FIG. 19B).

(c) Under isoflurane anesthesia, each PC-3 tumor mouse received an injection via the tail vein of approximately 5.5 MBq (150 mCi) of ⁶⁴Cu-NOTA-RGD, ⁶⁴Cu-NOTA-bombesin, ⁶⁴Cu-NOTA-RGD-bombesin, ⁶⁴Cu-NOTA-RGD (75 mCi) plus ⁶⁴Cu-NOTA-bombesin (75 mCi), or ⁶⁴Cu-DOTA-RGD-bombesin. Five-minute static PET images were acquired at 30 min, 1 h, and 4 h after injection of each tracer (n=4/group), and 10-min static PET images were acquired at 20 h. The images were reconstructed using a 2-dimensional ordered-subsets expectation maximum algorithm without attenuation or scatter correction.

Under isoflurane anesthesia, each 4T1 tumor mouse received an injection via the tail vein of 3.7 MBq (100 mCi) of ⁶⁴Cu-NOTA-RGD, ⁶⁴Cu-NOTA-BBN, or ⁶⁴Cu-NOTA-RGD-BBN. Five-minute static PET images were then acquired at 2 h after injection (n=3/group).

A series of blocking studies was also performed to validate the in vivo dual-receptor binding affinity of ⁶⁴Cu-NOTA-RGD-BBN in PC-3 tumor-bearing nude mice at 1 h after injection of about 5.5 MBq (150 mCi) of tracer (n=3/group). For each small-animal PET scan, regions of interest were drawn over each tumor, over normal tissue, and over major organs using vendor software (ASI Pro, version 5.2.4.0) on decay-corrected whole-body coronal images. The maximum radioactivity concentration (accumulation) within a tumor or an organ was obtained from the mean pixel values within the multiple-region-of-interest volume, which were converted to MBq/mL/min using a conversion factor. Assuming a tissue density of 1 g/mL, the regions of interest were converted to MBq/g/min and then divided by the administered activity to obtain an imaging region-of-interest-derived percentage injected dose (% ID)/g.

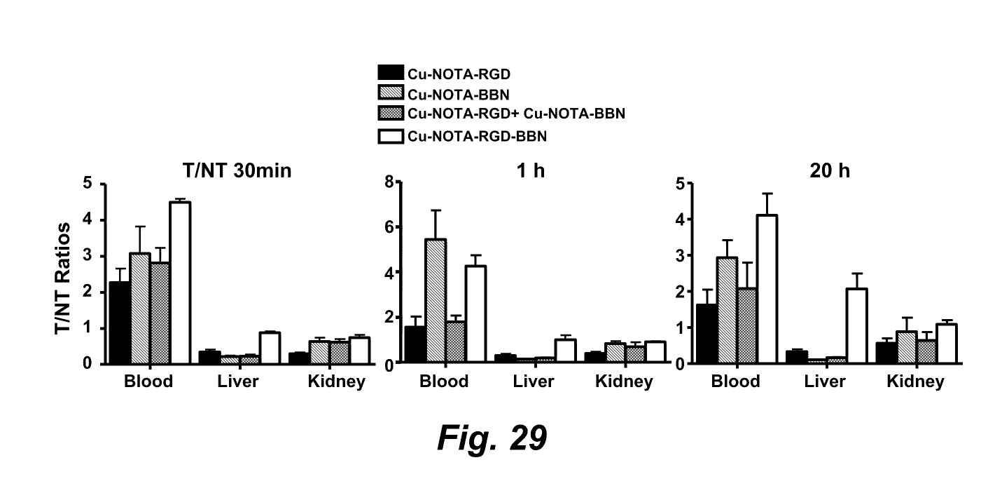

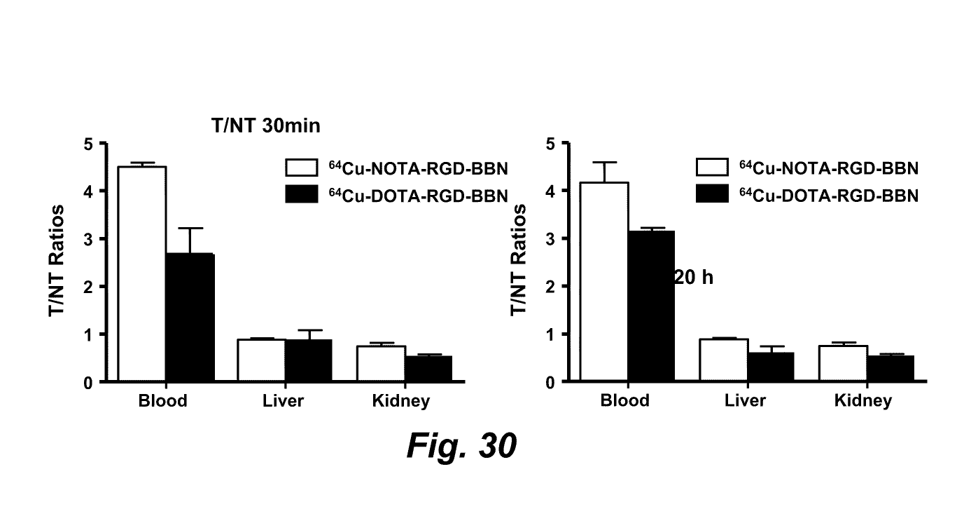

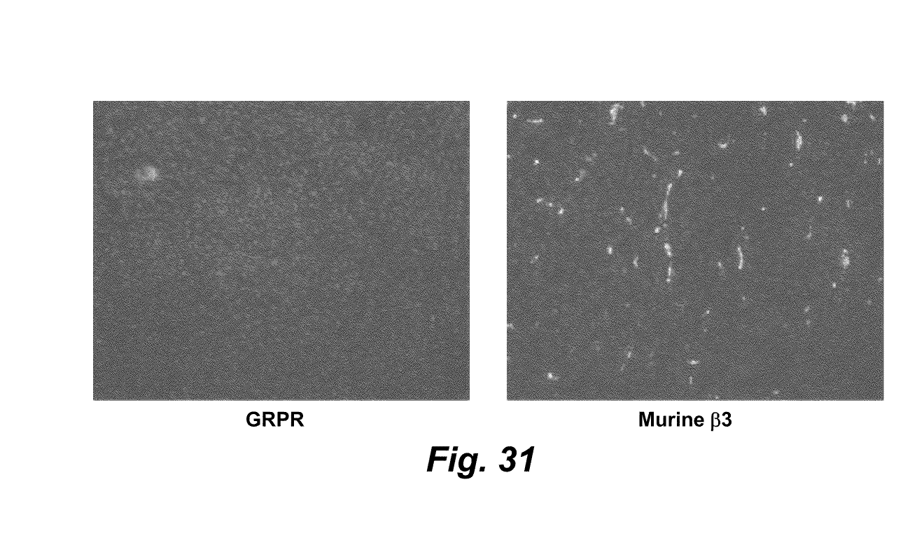

All tumors were clearly visible after injection of the different tracers, with high contrast to contralateral background at all time points measured from 30 min to 20 h, except for ⁶⁴Cu-NOTA-RGD (FIG. 24). ⁶⁴Cu-NOTA-RGD showed relatively low tumor uptake in PC-3 tumors because of the low expression of integrin αᵥβ₃ in PC-3 tumor tissue and the low affinity of monomeric RGD peptide with integrin receptor. Mice receiving ⁶⁴Cu-NOTA-BBN or ⁶⁴Cu-NOTA-RGD plus ⁶⁴Cu-NOTA-RGD showed a predominantly intestinal accumulation of the activity. Prominent kidney uptake of ⁶⁴Cu-NOTA-RGD-BBN and ⁶⁴Cu-DOTA-RGD-BBN at early time points was observed, suggesting that the peptide heterodimer tracers are excreted mainly through the kidneys. The quantified tumor and major organ uptake of the tracers is depicted in Table 2, below, and the clearance curves and tumor to non-tumor ratios are compared in FIGS. 29 and 30.

TABLE 2
Small-animal PET Data of ⁶⁴Cu-labeled Tracers on PC-3 Mice

| | Tissue | | | | |
|---|---|---|---|---|---|
| Tracer | or organ | 30 min | 1 h | 4 h | 20 h |
| ⁶⁴Cu-NOTA-RGD | Blood | 0.44 ± 0.15 | 0.43 ± 0.32 | 0.30 ± 0.11 | 0.34 ± 0.18 |
| | Liver | 2.86 ± 1.06 | 2.68 ± 1.15 | 2.15 ± 0.79 | 1.67 ± 0.69 |
| | Kidney | 3.37 ± 0.89 | 2.13 ± 0.85 | 1.43 ± 0.68 | 0.98 ± 0.48 |
| | Tumor | 1.01 ± 0.29 | 0.83 ± 0.24 | 0.66 ± 0.21 | 0.55 ± 0.32 |
| ⁶⁴Cu-NOTA-BBN | Blood | 0.74 ± 0.36 | 0.23 ± 0.11 | 0.19 ± 0.10 | 0.15 ± 0.05 |
| | Liver | 10.45 ± 2.27 | 8.85 ± 1.61 | 6.35 ± 0.57 | 4.20 ± 0.53 |
| | Kidney | 3.55 ± 1.17 | 1.50 ± 0.39 | 0.76 ± 0.45 | 0.50 ± 0.44 |
| | Tumor | 2.28 ± 0.35 | 1.25 ± 1.03 | 0.56 ± 0.40 | 0.44 ± 0.39 |
| ⁶⁴Cu-NOTA-RGD plus | Blood | 0.79 ± 0.24 | 0.71 ± 0.23 | 0.31 ± 0.26 | 0.26 ± 0.18 |
| ⁶⁴Cu-NOTA-BBN | | | | | |
| | Liver | 9.64 ± 4.15 | 6.74 ± 2.86 | 5.48 ± 0.84 | 3.24 6 0.78 |
| | Kidney | 3.58 ± 0.98 | 1.83 ± 1.01 | 1.57 ± 0.87 | 0.84 ± 0.60 |
| | Tumor | 2.22 ± 0.41 | 1.27 ± 0.50 | 0.87 ± 0.35 | 0.54 ± 0.39 |
| ⁶⁴Cu-NOTA-RGD-BBN | Blood | 0.68 ± 0.03 | 0.66 ± 0.15 | 0.49 ± 0.07 | 0.50 ± 0.15 |
| | Liver | 3.46 ± 0.26 | 2.80 ± 1.15 | 1.83 ± 0.68 | 0.98 ± 0.40 |
| | Kidney | 4.09 ± 0.81 | 3.06 ± 0.25 | 2.30 ± 0.41 | 1.87 ± 0.41 |
| | Tumor | 3.06 ± 0.11 | 2.78 ± 0.56 | 2.21 ± 0.49 | 2.04 ± 0.35 |
| ⁶⁴Cu-DOTA-RGD-BBN | Blood | 1.15 ± 0.49 | 0.69 ± 0.24 | 0.45 ± 0.20 | 0.31 ± 0.13 |
| | Liver | 3.40 ± 1.42 | 3.05 ± 1.07 | 2.33 ± 0.88 | 1.74 ± 0.90 |
| | Kidney | 5.99 ± 1.61 | 3.40 ± 1.25 | 2.51 ± 0.47 | 1.64 ± 0.34 |
| | Tumor | 3.05 ± 0.56 | 1.87 ± 0.41 | 1.05 ± 0.49 | 0.97 ± 0.24 |

Injection dose was about 5.5 Mbq (150 mCi) per mouse. Data are % ID/g±SD (n=4/group).

For ⁶⁴Cu-NOTA-RGD, ⁶⁴Cu-NOTA-BBN, ⁶⁴Cu-NOTA-RGD plus ⁶⁴Cu-NOTA-BBN, and ⁶⁴Cu-NOTA-RGD-BBN, the tracers cleared rapidly from the blood, with less than 1% ID/g remaining at 30 min after injection. ⁶⁴Cu-NOTA-RGD-BBN showed slightly lower blood clearance than the other tracers, whereas ⁶⁴Cu-NOTA-BBN cleared the most rapidly. The tumor uptake of ⁶⁴Cu-NOTARGD-BBN was determined to be 3.06±0.11, 2.78±0.56, 2.21±0.49, and 2.04±0.35% ID/g at 0.5, 1, 4, and 20 h after injection, respectively-significantly higher than all the other tracers tested (P, 0.01, n=4/group, Table 2).

Tumor uptake was about the same for ⁶⁴Cu-NOTA-RGD, ⁶⁴Cu-NOTA-BBN, and ⁶⁴Cu-NOTA-RGD plus ⁶⁴Cu-NOTA-BBN from 1 to 20 h after injection. ⁶⁴Cu-NOTA-RGD-BBN showed higher kidney uptake than the other tracers at any time examined. ⁶⁴Cu-NOTA-RGD and ⁶⁴Cu-NOTA-RGD plus ⁶⁴Cu-NOTA-BBN showed a similar clearance curve in the kidneys, whereas the kidney uptake of ⁶⁴Cu-NOTA-BBN was the lowest at all time points. ⁶⁴Cu-NOTA-BBN and ⁶⁴Cu-NOTA-RGD plus ⁶⁴Cu-NOTA-BBN exhibited predominantly liver uptake, whereas uptake of ⁶⁴Cu-NOTA-RGD-BBN and ⁶⁴Cu-NOTA-RGD in the liver was relatively low. The tumor to non-tumor ratios of ⁶⁴Cu-NOTA-RGD-BBN were significantly higher than those of the other tracers at 4 h after injection (P, 0.05, FIG. 29), mostly because of the high tumor uptake of ⁶⁴Cu-NOTA-RGD-BBN. The pancreas could not be delineated on small-animal PET because of the limit of the spatial resolution.

The tumor and major organ uptake and tumor to non-tumor ratios of ⁶⁴Cu-NOTA-RGD-BBN and ⁶⁴Cu-DOTA-RGD-BBN are directly compared in FIG. 30. For blood and kidneys, the 2 tracers showed almost identical clearance curves. The tumor uptake of ⁶⁴Cu-NOTA-RGD-BBN was much higher than that of ⁶⁴Cu-DOTA-RGD-BBN after 1 h after injection. For example, at 4 h after injection, the tumor uptake was 2.21±0.49% ID/g and 1.05±0.49% ID/g for ⁶⁴Cu-NOTA-RGD-BBN and ⁶⁴Cu-DOTA-RGD-BBN, respectively (P, 0.05, n=4/group, Table 2). Liver uptake was similar for the 2 tracers at 30 min after injection, but liver uptake of ⁶⁴Cu-NOTA-RGD-BBN became gradually lower than that of ⁶⁴Cu-DOTA-RGD-BBN. Because of the similar normal-organ uptake and much higher tumor uptake of ⁶⁴Cu-NOTA-RGD-BBN, the tumor to non-tumor ratios of ⁶⁴Cu-NOTA-RGD-BBN were all higher than those of ⁶⁴Cu-DOTA-RGD-BBN after the 1-h time point (P, 0.05, n=4/group).

The in vivo behaviors of ⁶⁴Cu-NOTA-RGD, ⁶⁴Cu-NOTA-BBN, and ⁶⁴Cu-NOTA-RGD-BBN were also tested in a murine 4T1 breast tumor model. The 4T1 tumor tissue expresses a moderate level of murine integrin b3 but undetectable GRPR. As shown in FIG. 25A, ⁶⁴Cu-NOTA-BBN had virtually no uptake in 4T1 tumors, whereas both ⁶⁴Cu-NOTA-RGD and ⁶⁴Cu-NOTA-RGD-BBN showed clear tumor contrast due to the integrin αᵥβ₃ recognition of RGD monomer and RGD-BBN heterodimer in vivo, respectively. At the 2-h time point, tumor uptake of ⁶⁴Cu-NOTA-RGD, 4Cu-NOTA-BBN, and ⁶⁴Cu-NOTA-RGD-BBN was 0.65±0.07, 0.33±0.18, and 1.88±0.28% ID/g, respectively (n=3/group, FIG. 25B).

The in vivo integrin and GRPR dual-receptor binding property of ⁶⁴Cu-NOTA-RGD-BBN was confirmed by several blocking studies (FIG. 26A). The tumor uptake of ⁶⁴Cu-NOTA-RGD-BBN was only partially inhibited by either RGD (from 2.78±0.56% ID/g to 1.64±0.77% ID/g), or BBN (from 2.78±0.56% ID/g to 0.76±0.36% ID/g). However, when ⁶⁴Cu-NOTA-RGD-BBN was co-administered with both RGD and BBN, the tumor uptake was almost totally blocked (0.54±0.41% ID/g) (FIG. 26B, n=3/group).

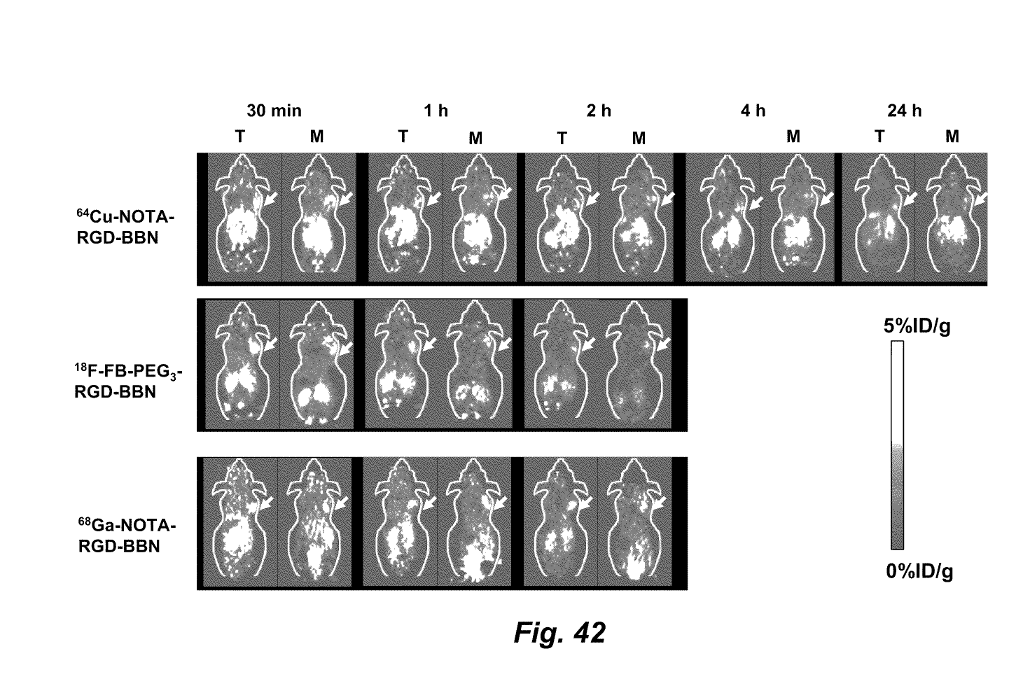

(c) Representative coronal microPET images of T47D and MDA-MB435 tumor-bearing mice (n=4/group) at different times after intravenous injection of 3.7-5.6 MBq (100-150 μCi) of ¹⁸F-FB-PEG₃-RGD-BBN, ⁶⁴Cu-NOTA-RGD-BBN or ⁶⁸Ga-NOTA-RGD-BBN are shown in FIG. 42. The tumors after injection of the tracers were all clearly visible with high contrast to contralateral background at all time points measured from 30 min. The mice injected with ⁶⁴Cu-NOTA-RGD-BBN showed relatively higher abdomen activity accumulation than those injected with ⁶8Ga-NOTA-RGD-BBN or ¹⁸F-FB-PEG₃-RGD-BBN. Prominent uptake of ¹⁸F-FB-PEG₃-RGD-BBN was observed in the kidneys at early time points, suggesting that this tracer is mainly excreted through the renal route. Quantification of tumor and major organ activity accumulation in the microPET scans was realized by measuring the regions of interest (ROIs) that encompassing the entire organ on the coronal images.

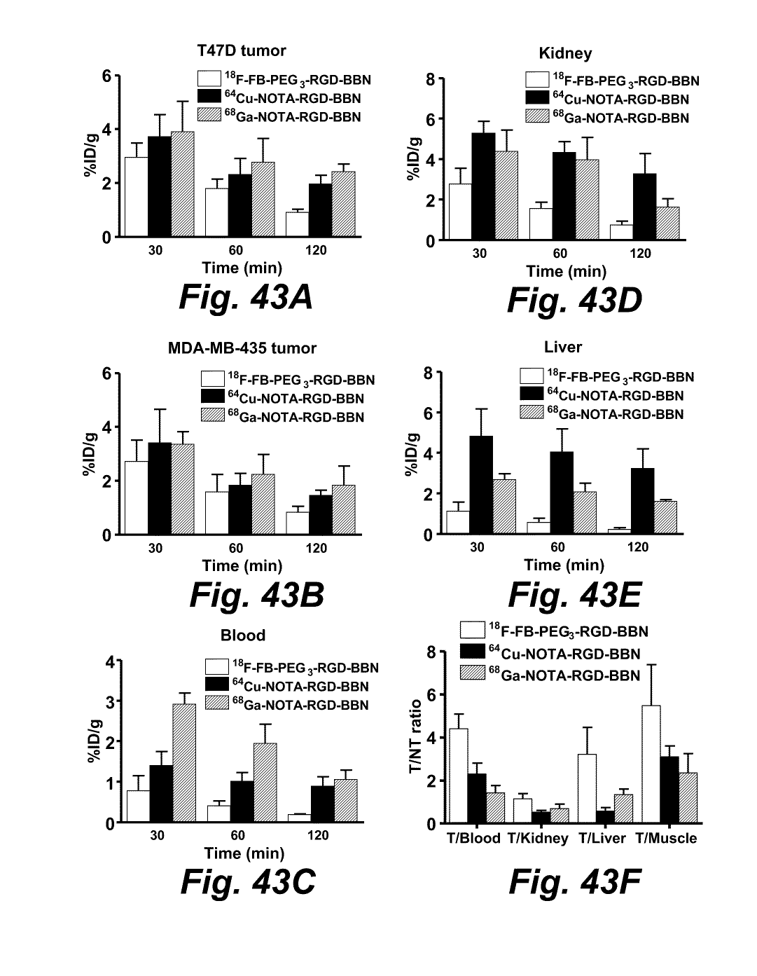

The tumor and major organ uptake comparison of ¹⁸F-FB-PEG₃-RGD-BBN, ⁶⁴Cu-NOTA-RGD-BBN and ⁶⁸Ga-NOTA-RGD-BBN are depicted in FIGS. 43A-43F and Table 3.

TABLE 3
Quantified MicroPET Imaging Data of ¹⁸F-FB-PEG₃-RGD-BBN, ⁶⁴Cu-NOTA-RGD-BBN,
and ⁶⁸Ga-NOTA-RGD-BBN in T47D and MDA-MB-435 Tumor-bearing Nude Mice
(Means ± SD, n = 4~8).

| | ¹⁸F-FB-PEG₃-RGD-BBN | | | ⁶⁸Ga-NOTA-RGD-BBN | | |
|---|---|---|---|---|---|---|
| | 0.5 h | 1 h | 2 h | 0.5 h | 1 h | 2 h |
| Blood | 0.78 ± 0.37 | 0.41 ± 0.11 | 0.18 ± 0.02 | 2.91 ± 0.28 | 1.95 ± 0.47 | 1.06 ± 0.23 |
| Liver | 1.13 ± 0.43 | 0.56 ± 0.21 | 0.23 ± 0.07 | 2.68 ± 0.29 | 2.08 ± 0.43 | 1.60 ± 0.09 |
| Kidney | 2.77 ± 0.78 | 1.56 ± 0.32 | 0.74 ± 0.19 | 4.39 ± 1.05 | 3.96 ± 1.12 | 1.64 ± 0.40 |
| Muscle | 0.81 ± 0.23 | 0.33 ± 0.17 | 0.15 ± 0.05 | 1.33 ± 0.28 | 1.18 ± 0.44 | 0.78 ± 0.07 |
| T47D | 2.96 ± 0.53 | 1.81 ± 0.34 | 0.91 ± 0.12 | 3.91 ± 1.13 | 2.78 ± 0.87 | 2.42 ± 0.29 |
| MDA-MB- | 2.72 ± 0.80 | 1.59 ± 0.65 | 0.84 ± 0.22 | 3.36 ± 0.47 | 2.24 ± 0.73 | 1.84 ± 0.72 |
| 435 | | | | | | |

| | ⁶⁴Cu-NOTA-RGD-BBN | | | | |
|---|---|---|---|---|---|
| | 0.5 h | 1 h | 2 h | 4 h | 24 h |
| Blood | 1.40 ± 0.34 | 1.01 ± 0.21 | 0.89 ± 0.23 | 0.54 ± 0.20 | 0.36 ± 0.04 |
| Liver | 4.82 ± 1.34 | 4.05 ± 1.12 | 3.22 ± 0.97 | 2.76 ± 0.81 | 2.24 ± 0.36 |
| Kidney | 5.29 ± 0.58 | 4.32 ± 0.55 | 3.28 ± 0.99 | 2.95 ± 0.26 | 2.10 ± 0.72 |
| Muscle | 0.91 ± 0.27 | 0.75 ± 0.12 | 0.56 ± 0.33 | 0.40 ± 0.07 | 0.21 ± 0.04 |
| T47D | 3.73 ± 0.81 | 2.33 ± 0.59 | 1.97 ± 0.32 | 1.94 ± 0.22 | 1.38 ± 0.16 |
| MDA-MB- | 3.42 ± 1.23 | 1.84 ± 0.44 | 1.47 ± 0.19 | 1.79 ± 0.20 | 1.29 ± 0.09 |
| 435 | | | | | |

The T47D or MDA-MB435 tumor uptake is expressed as the average of each tracer in four mice, while the normal organ uptake is expressed as the average of each tracer in eight mice (four T47D tumor-bearing mice and four MDA-MB-435 tumor-bearing mice per tracer). As shown in FIGS. 43A-43F, for all three tracers, the T47D tumor uptake was higher than the MDA-MB-435 tumor uptake at any time examined, which is consistent with the in vitro cell uptake studies. For example, the tumor uptake comparison of ¹⁸F-FB-PEG₃-RGD-BBN in T47D tumor and MDA-MB-435 was 2.96±0.53 vs 2.72±0.80, 1.81±0.34 vs 1.59±0.65 and 0.91±0.12 versus 0.84±0.22% ID/g at 30 min, 60 min, and 120 min p.i, respectively (FIGS. 43A and 43B). The tumor uptake of ⁶⁸Ga-NOTA-RGD-BBN was relatively higher than that of the ¹⁸F and ⁶⁴Cu labeled RGD-BBN tracers from 30 min to 120 min p.i and the differences were significant comparing with ¹⁸F-FB-PEG₃-RGD-BBN at any time tested (n=4, P<0.05). ¹⁸F-FB-PEG₃-RGD-BBN also showed rapid wash out in blood and normal organs. As can be seen from FIGS. 43A-43F, the uptake of the ¹⁸F labeled RGD-BBN tracer decreased rapidly with time in blood, kidney and liver. The uptake values of ¹⁸F-FB-PEG₃-RGD-BBN in blood and normal organs were all significantly lower than those of the ⁶⁴Cu and ⁶⁸Ga tracers at any time from 30 min to 120 min (n=8, P<0.05). ⁶⁸Ga-NOTA-RGD-BBN also showed higher blood retention as comparing with the ¹⁸F and ⁶⁴Cu tracers (FIG. 43C). The kidney uptake of ⁶⁸Ga-NOTA-RGD-BBN and ⁶⁴Cu-NOTA-RGD-BBN decreased with time and ⁶⁸Ga-NOTA-RGD-BBN seemed to be cleared slightly more rapidly than ⁶⁴Cu-NOTA-RGD-BBN. At 120 min p.i, the kidney uptake was 0.74±0.19% ID/g for ¹⁸F-FB-PEG₃-RGD-BBN, 1.64±0.40% ID/g for ⁶⁸Ga-NOTA-RGD-BBN and 3.28±0.99% ID/g for ⁶⁴Cu-NOTA-RGD-BBN, respectively (n=8, FIG. 43D).

The liver uptake of ⁶⁴Cu-NOTA-RGD-BBN was significantly higher than those of the 18F and ⁶⁸Ga labeled RGD-BBN tracers at any time points examined (n=8, P<0.05). At 4 h and 24 h p.i, the liver uptake of ⁶⁴Cu-NOTA-RGD-BBN was still higher than that of the tumor uptake (Table 3). The liver uptake of ¹⁸F-FB-PEG₃-RGD-BBN was very low at any time with the highest uptake being 1.13±0.43% ID/g at 30 min p.i, indicating the ¹⁸F labeled RGD-BBN tracer was almost not excreted from the hepatobiliary route (FIG. 43E). Although the absolute tumor uptake of ¹⁸F-FB-PEG₃-RGD-BBN was lower than that of the other two tracers, the tumor to non-tumor (T/NT) ratios of ¹⁸F-FB-PEG₃-RGD-BBN were all significantly higher than those of ⁶⁴Cu-NOTA-RGD-BBN and ⁶⁸Ga-NOTA-RGD-BBN (P<0.05), due to the rapid wash out of the tracer in blood and normal organs (FIG. 43F).

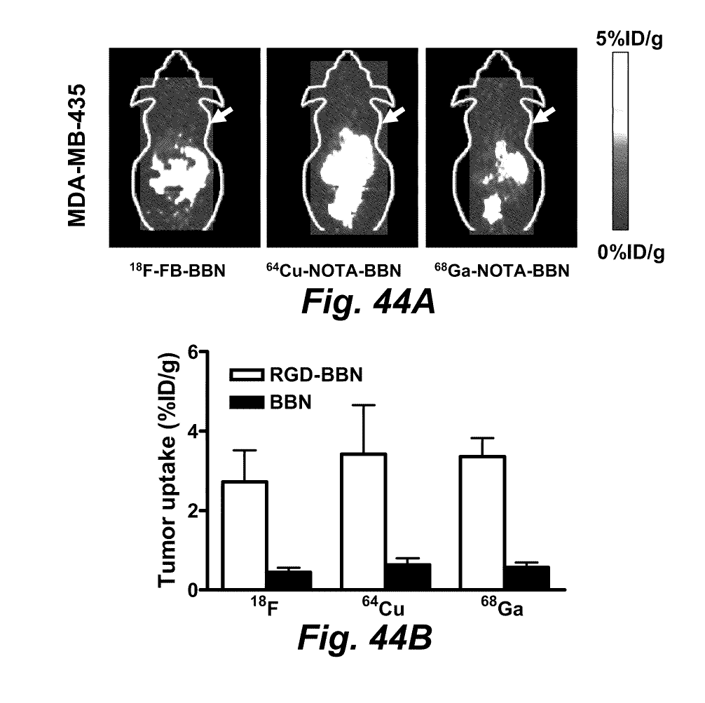

¹⁸F, ⁶⁴Cu and ⁶⁸Ga labeled BBN tracers were also tested in the nude mice bearing MDA-MB-435 tumor, which was integrin αᵥβ₃-positive, but GRPR-negative (FIG. 40B). As shown in FIG. 44A, the BBN tracers appeared to be more lipophilic than the corresponding RGD-BBN tracers, resulting in significant activity accumulation in gallbladder and intestines. Because the absent expression of the GRPR, none of ¹⁸F-FB-BBN, ⁶⁴Cu-NOTA-RGD-BBN and ⁶⁸Ga-NOTA-RGD-BBN was able to visualize the tumors. The MDA-MB-435 tumor uptake at 30 min p.i was 0.45±0.11% ID/g for ¹⁸F-FB-BBN, 0.63±0.17% ID/g for ⁶⁴Cu-NOTA-RGD-BBN and 0.57±0.13% ID/g for ⁶⁸Ga-NOTA-RGD-BBN, which was significantly lower than the corresponding RGD-BBN radiotracers (P<0.01, FIG. 44B).

### Example 17

### Metabolic Stability of ¹⁸F-FB-BBN-RGD

A PC-3 tumor mouse was injected intravenously with 3.7 MBq of ¹⁸F-FB-BBN-RGD. At 1 hr after injection, the mouse was sacrificed, the blood, urine, liver, kidneys, and tumor were collected, and metabolite analysis was performed as reported previously (Wu et al., (2007) Eur. J. Nucl. Med. Imaging 34: 1823-1831). In brief, the blood sample was immediately centrifuged for 5 min at 13,200 rpm. Other tissues were homogenized and then centrifuged for 5 min at 13,200 rpm. Each supernatant was passed through a C18 Sep-Pak cartridge. The urine sample was diluted directly with 1 mL of PBS and passed through a C18 Sep-Pak cartridge. The cartridges were each washed with 2 mL of water and eluted with 2 mL of ACN containing 0.1% TFA. The ACN eluent was concentrated and injected onto the analytic HPLC system. The eluant was collected with a fraction collector (0.5 min/fraction), and the radioactivity of each fraction was measured with a 7-counter.

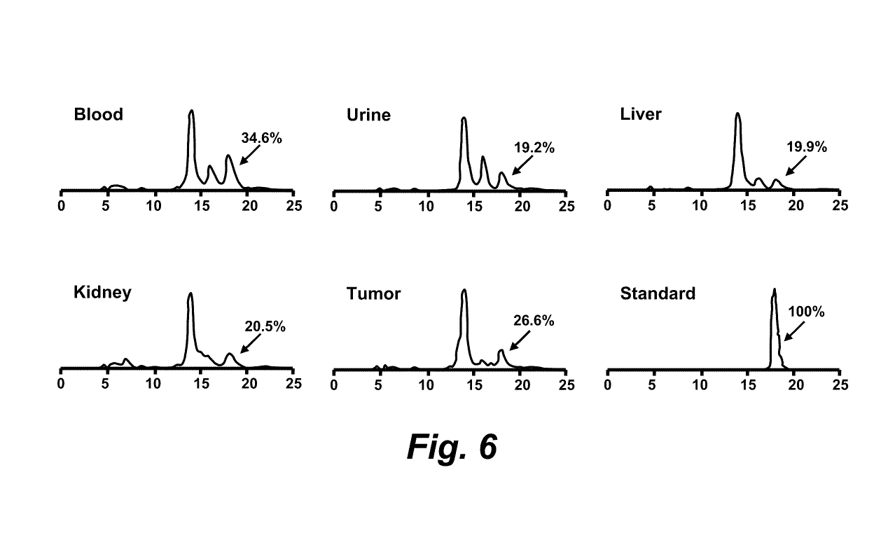

The metabolic stability of ¹⁸F-FB-BBN-RGD was determined in mouse blood, urine, liver, kidneys, and tumor homogenates at 60 min after injection. The extraction efficiencies were 91.4% for blood, 73.6% for liver, 95.2% for kidneys, and 94.6% for PC-3 tumor, respectively. The elution efficiencies of the soluble fractions were 93.2% for blood, 68.6% for liver, 89.1% for kidneys, and 90.0% for PC-3 tumor. HPLC analysis results of the ACN-eluted fractions were shown in FIG. 6. The average fraction of intact tracer was from 19.2% to 34.6% (Table 4).

TABLE 4
Extraction Efficiency, Elution Efficiency, and HPLC Analysis
of Soluble Fractions of Tissue Homogenates at 1 Hour After
Injection of ¹⁸F-FB-BBN-RGD

| Fraction | Blood | Urine | Liver | Kidney | PC-3 tumor |
|---|---|---|---|---|---|
| | Extraction efficiency (%) | | | | |
| Insoluble | 8.6 | ND | 26.4 | 4.8 | 5.4 |
| Soluble | 91.4 | ND | 73.6 | 95.2 | 94.6 |
| | Elution efficiency | | | | |
| Unretained | 5.2 | 0.1 | 28.2 | 9.6 | 8.3 |
| Wash water | 1.6 | 0.2 | 3.2 | 1.3 | 1.7 |
| ACN eluent | 93.2 | 99.7 | 68.6 | 89.1 | 90.0 |
| | HPLC analysis (%) | | | | |
| Intact tracer | 34.6 | 19.2 | 19.9 | 20.5 | 26.6 |

Although we did not identify the composition of the metabolites, we found that all metabolites came off the HPLC column earlier than those for the parent compound. No defluoridation of ¹⁸F-FB-BBN-RGD was observed, as no visible bone uptake was found on any of the small-animal PET scans. Overall, ¹⁸F-FB-BBN-RGD exhibited comparable metabolic stability with ¹⁸F-FB-BBN (5).

¹⁸F-FB-PEG₃-RGD-BBN, ⁶⁴Cu-NOTA-RGD-BBN, or ⁶⁸Ga-NOTA-RGD-BBN were incubated in fetal bovine serum (FBS) for 2 h at room temperature to test the in vitro serum stability. After passing through a 0.22-μm Millipore filter, the samples were analyzed by radio-HPLC. For metabolism studies, female nude mice (n=2/group) were injected with ¹⁸F-FB-PEG₃-RGD-BBN, ⁶⁴Cu-NOTA-RGD-BBN, or ⁶⁸Ga-NOTA-RGD-BBN at a dose of 7.4 MBq (200 μCi) in 0.2 mL PBS via tail vein. At 60 min p.i., the urine samples were collected and then centrifuged at 8,000 rpm for 5 min. The supernatant was collected, filtered through a 0.22-μm Millipore filter, and then analyzed by radio-HPLC.

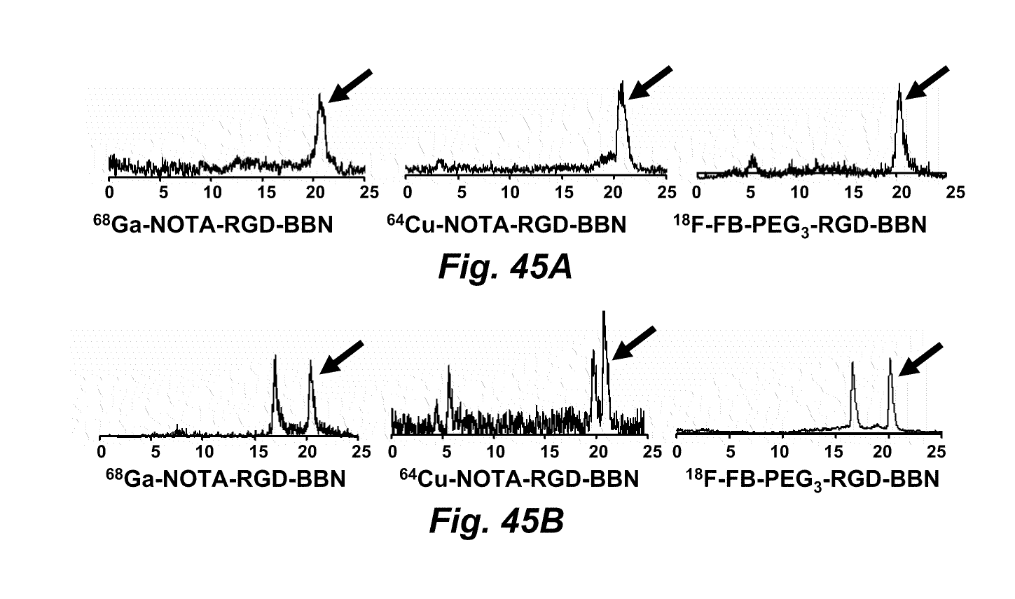

The serum stability of ¹⁸F-FB-PEG₃-RGD-BBN, ⁶⁴Cu-NOTA-RGD-BBN, and ⁶⁸Ga-NOTA-RGD-BBN was tested by incubating with FBS for 2 h at room temperature. As shown in FIG. 45A, all three tracers showed good in vitro serum stability, with only minor peaks can be seen around Rt 5 min for ¹⁸F-FB-PEG₃-RGD-BBN, and ⁶⁴Cu-NOTA-RGD-BBN as detected by radio-HPLC.

The metabolic stability of the three tracers in mice urine at 60 min after injection was also studied. As shown in FIG. 45B, all the three tracers showed detectable metabolites in the urine. Although we did not identify the composition of the metabolites, it was found that all metabolites came off the HPLC column earlier than those for the parent compounds. The major metabolite peaks were found at about 17 min for ⁶⁸Ga-NOTA-RGD-BBN and ¹⁸F-FB-PEG₃-RGD-BBN, and about 20 min for ⁶⁴Cu-NOTA-RGD-BBN. Because the RGD peptides seemed to be more metabolically stable than BBN peptides in urine, it is possible that the metabolites of the RGD-BBN radiotracers were more likely to be from the BBN counterparts of the radiolabeled heterodimers.

### Example 18

### Biodistribution Studies

Male athymic nude mice bearing PC-3 xenografts were injected with 0.74 MBq (20 μCi) of ⁶⁸Ga-NOTA-RGD-BBN to evaluate the distribution of the tracer in the tumor tissues and major organs. At 0.5 h and 1 h after injection of the tracer, the tumor-bearing mice were killed and dissected. Blood, tumor, major organs, and tissues were collected and wet-weighed. The radioactivity in the tissue was measured by y counter (Packard). The results are presented as percentage injected dose per gram of tissue (% ID/g). For each mouse, the radioactivity of the tissue samples was calibrated against a known aliquot of the injectate and normalized to a body mass of 20 g. Values were expressed as mean±SD for groups of four animals (n=4 per group).

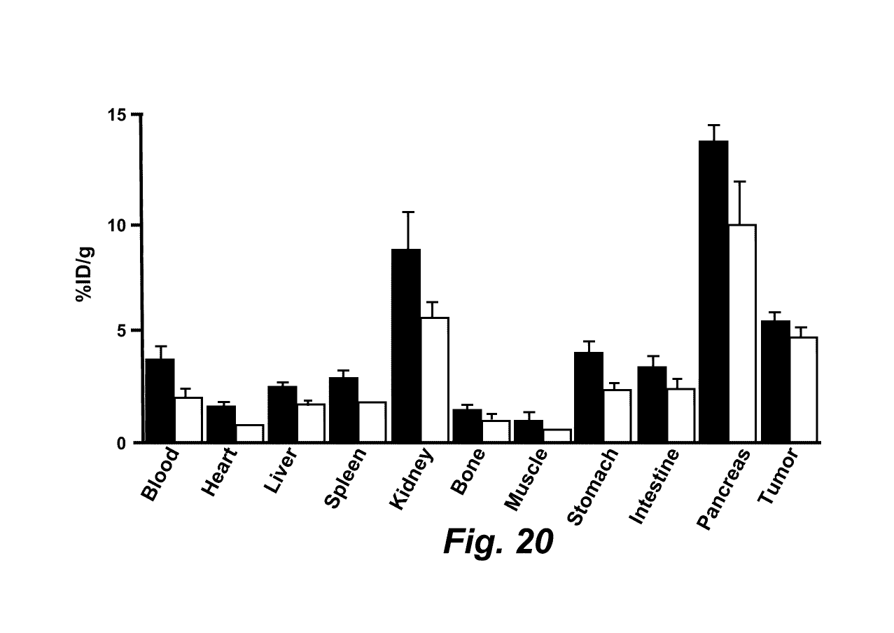

The biodistribution study of ⁶⁸Ga-NOTA-RGD-BBN was performed in nude mice bearing PC-3 tumors. Each mouse was injected with 0.74 MBq (20 μCi) of ⁶⁸Ga-NOTA-RGD-BBN and then killed at 0.5 h and 1 h after injection (n=4 per group). As shown graphically in FIG. 20, the tracer uptake decreased from 0.5 h to 1 h in the PC-3 tumors and all the examined organs. For example, the tumor uptake was 5.43±0.54% ID/g at 0.5 h and 4.76±0.46% ID/g at 1 h. The kidney uptake decreased from 8.76±1.83% ID/g at 0.5 h after injection to 5.58±0.75% ID/g at 1 h after injection. The tumor uptake of ⁶⁸Ga-NOTA-RGD-BBN was significantly higher than that in the blood and normal organs, such as the heart, liver, spleen, bone, and muscle at 0.5 h and 1 h (p<0.01). The tracer also showed high uptake in the pancreas, stomach and intestine due to the high GPPR expression in these organs.

Normal BALB/c mice received an injection via the tail vein of 370 kBq (10 mCi) of ⁶⁴Cu-NOTA-RGD-bombesin to evaluate the distribution of the tracer. The blocking experiments were also performed by co-injection of ⁶⁴Cu-NOTA-RGD-bombesin with a saturating dose of c(RGDyK) (10 mg/kg of mouse body weight), bombesin (15 mg/kg), or RGD (10 mg/kg) plus bombesin (15 mg/kg). All mice were sacrificed at 1 h after injection of the tracer. Blood, tumor, and major organs and tissues were collected and wet-weighed. Stomach and intestines were cleaned of their contents in this experiment. The radioactivity in the tissue was measured using a g-counter (Packard). The results were presented as percentage injected dose per gram of tissue (% ID/g). Values were expressed as mean 6 SD (n=4/group).

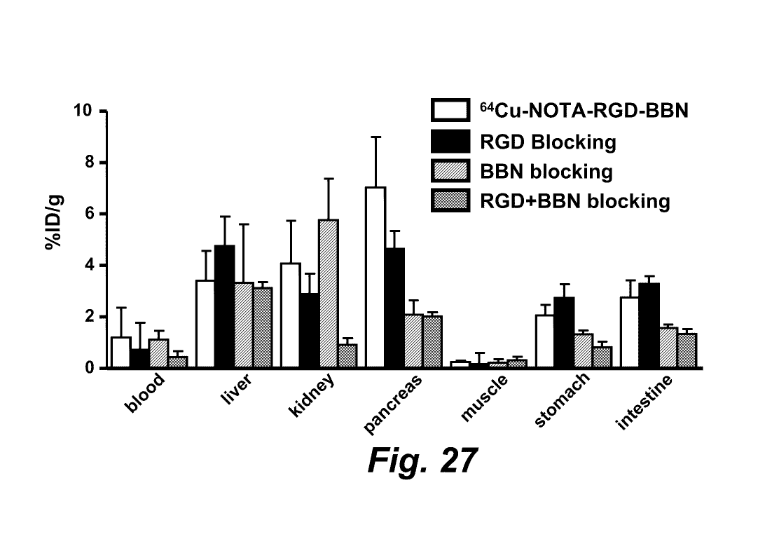

The biodistribution of ⁶⁴Cu-NOTA-RGD-BBN (370 kBq/mouse) was examined in normal BALB/c mice. The blocking experiments were also performed by co-injecting ⁶⁴Cu-NOTA-RGD-BBN with a saturating dose of RGD, BBN, or RGD plus BBN and then sacrificing the mice at 1 h after injection (n=4/group). As shown in FIG. 27, the pancreas had predominant uptake of ⁶⁴Cu-NOTA-RGD-BBN at 1 h after injection because of the high GRPR expression of this organ (Hoffman et al., (2003) J. Nucl. Med. 44: 823-831). In the presence of a blocking dose of BBN or RGD plus BBN, pancreatic uptake of the tracer decreased significantly from 7.03±1.96% ID/g to 2.08±0.57% ID/g (BBN blocking) and 2.01±0.18% ID/g (RGD-plus-BBN blocking), respectively (P, 0.01), indicating GRPR specific targeting of ⁶⁴Cu-NOTA-RGD-BBN in vivo. Blood uptake of ⁶⁴Cu-NOTA-RGD-BBN decreased after co-injection of RGD or RGD plus BBN, probably because the excess dose of RGD increased clearance of the tracer. Liver uptake was unaffected by blocking with BBN or both RGD plus BBN but slightly increased after blocking with RGD. Stomach and intestinal uptake of the tracer decreased after blocking with BBN or RGD plus BBN but slightly increased after blocking with RGD.

### Example 19

### Immunofluorescence Staining

Immunofluorescence staining studies were performed as described in Wu et al., (2007) Eur. J. Nucl. Med. Mol. Imaging. 34: 1823-1831, incorporated herein by reference in its entirety, with some modifications. Briefly, frozen PC-3 tumor and organ tissue slices (5-μm thickness) from the tumor-bearing nude mice were fixed with ice-cold acetone, rinsed with PBS and blocked with 10% goat serum for 30 min at room temperature. The slices were incubated with goat anti-GRPR antibody (1:100; Santa Cruz Biotechnology, Santa Cruz, Calif.), humanized anti-human integrin αᵥβ₃ antibody Abegrin (Wu et al., (2007) Eur. J. Nucl. Med. Mol. Imaging. 34: 1823-1831) (20 μg/ml), or hamster anti-β₃ antibody (1:100; BD Biosciences, San Jose, Calif.) for 1 h at room temperature, and then visualized with FITC-conjugated donkey antigoat, Cy3-conjugated donkey antihuman or Cy3-conjugated goat anti-hamster secondary antibodies (1:200; Jackson ImmunoResearch Laboratories, West Grove, Pa.), respectively. For the overlaid staining of CD31 and murine P3, PC-3 tumor slices were incubated with rat anti-mouse CD31 antibody (1:100; BD Biosciences) and hamster anti-β₃ antibody (1:100; BD Biosciences) and then visualized with Cy3-conjugated goat anti-rat and FITC-conjugated goat anti-hamster secondary antibody (1:200; Jackson ImmunoResearch Laboratories).

The expression of GRPR and integrin αᵥβ₃ in the PC-3 tumor and normal organs was tested by immunofluorescent staining using anti-GRPR, anti-human αᵥβ₃ and antimurine β₃ antibodies. PC-3 tumors were found to be positive for GRPR, human αᵥβ₃ and murine β₃ (FIG. 21a). Because RGD peptide can bind both human and murine integrin, while BBN can bind GRPR, PC-3 tumors possess recognition sites for both RGD and BBN. In contrast, normal organs such as the liver, kidneys and muscle do not express GRPR, while the small intestine and stomach express high levels of GRPR around the lumen. Some other normal organs also express low levels of murine β₃. Overlaid staining of CD31 and murine β₃ in PC-3 tumors is shown in FIG. 21b. Most of the murine integrin β₃-positive areas were also CD31-positive, indicating that the expression of murine integrin αᵥβ₃ was derived from the tumor vasculature.

The expression of GRPR, human integrin αᵥβ₃ and murine integrin β₃ on T47D and MDA-MB435 tumor tissues were detected by immunofluorescent staining. Briefly, frozen T47D and MDA-MB435 tumor slices (5-mm thickness) from the tumor-bearing nude mice were fixed with ice-cold acetone, rinsed with PBS and blocked with 10% goat serum for 30 min at room temperature. The slices were incubated with goat anti-GRPR antibody (1:100; Santa Cruz Biotechnology, Santa Cruz, Calif.), humanized anti-human integrin αᵥβ₃ antibody (ABEGRIN™, 20 μg/mL) (38), or hamster anti-β₃ antibody (1:100; BD Biosciences, San Jose, Calif.) for 1 h at room temperature and then visualized with FITC-conjugated donkey anti-goat, Cy3-conjugated donkey anti-human and FITC-conjugated goat anti-hamster secondary antibodies (1:200; Jackson Immuno-Research Laboratories, West Grove, Pa.), respectively.

The expression of GRPR and integrin αᵥβ₃ in the T47D and MDA-MB435 tumor tissues was detected by immunofluorescent staining. As shown in FIG. 40C, T47D tumor showed strong GRPR staining, while MDA-MB435 tumor had only weak and background staining, which is consistent with the cell-based radioligand study (FIG. 40A). Because the anti-integrin αᵥβ₃ antibody ABEGRIN-used only recognizes the human integrin αᵥβ₃, but does not cross-react with murine integrin αᵥβ₃, the positive staining using ABEGRIN™ as the first antibody reflected the human integrin αᵥβ₃ expression of the tumor cells. MDA-MB435 tumor tissue showed higher human integrin αᵥβ₃ expression, and T47D tumor tissue showed lower human integrin αᵥβ₃ expression due to the different integrin αᵥβ₃ level of the tumor cells. Besides human integrin αᵥβ₃ expressed by the tumor cells, the tumors grown in the nude mice also expressed murine integrin αᵥβ₃ during the tumor angiogenesis. As shown in FIG. 40C, both T47D and MDA-MB435 tumors expressed murine integrin β₃, which can also be recognized by RGD.

### Example 20

### Synthesis and Radiolabeling of ¹⁸F-PEG₃-Glu-RGD-BBN

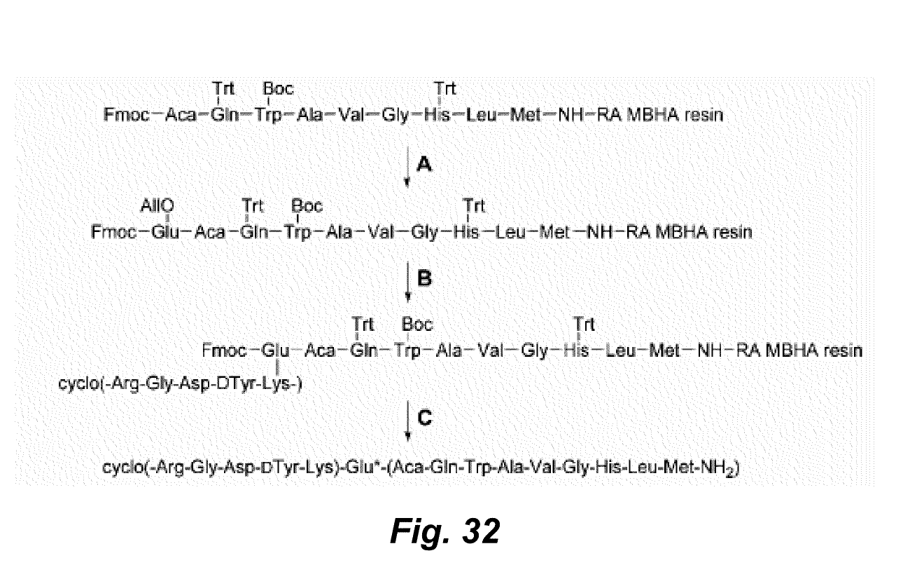

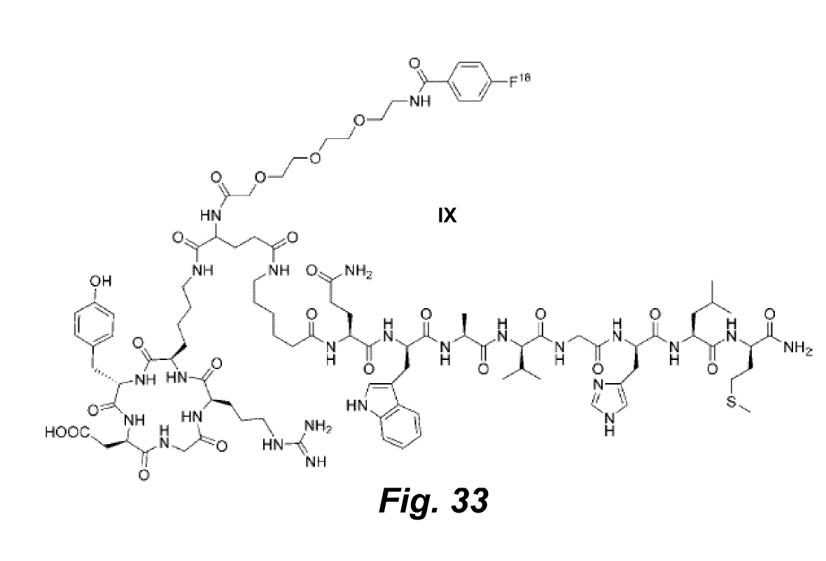

A Glu-RGD-BBN peptide heterodimer was synthesized step-wise by solid-phase peptide synthesis method, illustrated in FIG. 32. PEG₃-Glu-RGD-BBN was synthesized following a previously reported procedure (Li et al., (2008) J. Nucl. Med. 49: 453-461, incorporated herein by reference in its entirety). ¹⁸F-FB-PEG₃-Glu-RGD-BBN (FIG. 33) was prepared by coupling N-succinimidyl-4-¹⁸F-fluorobenzoate (¹⁸F-SFB) with PEG₃-Glu-RGD-BBN under slightly basic condition at 60° C. for 30 min followed by HPLC purification. The radiochemical yield was 42% from ¹⁸F-SFB with high radiochemical purity (>99%). The effective specific activity was estimated to be 100 TBq/mmol on the basis of the labeling agent ¹⁸F-SFB, as the unlabeled peptides were efficiently separated from the product.

### Synthesis of Glu-RGD-BBN

Loading of Fmoc-Met-Rink Amide MBHA resin, synthesis of the bombesin peptide follows standard peptide synthesis protocols. Side chain protection was trityl (Trt) for histidine (His) and glutamine (Gln) and tert-butoxycarbonyl (Boc) for tryptophan (Trp). After loading Fmoc-Glu-OAII onto Aca, the α-allyl ester was then removed by treatment with Pd(Ph₃P)₄/CHCl₃/AcOH/NMM. The α-carboxylate was activated and coupled with cyclic RGD peptide cyclo(Arg-Gly-Asp-DTyr-Lys) (RGD) via the lysine side chain ε-amine group. After removing the Fmoc from Glu, the final peptide cyclo[Arg-Gly-Asp-D-Tyr-Lys(Glu*-Aca-Gln-Trp-Ala-Val-Gly-His-Leu-Met-NH₂)] (Glu-RGD-BBN) was obtained by detaching/deprotecting the Rink Amide-MBHA resin using 95% TFA in dichloromethane (DCM) plus ethandithiol (EDT) and triisopropylsilane (TIS) as scavengers. ES-MS: m/z 1783.9 for [M+H]⁺ (C₈₁H₁₂₃N₂₄O₂₀S, calcd. 1783.9). RP-HPLC Rₜ=18.6 min.

### Synthesis of PEG₃-Glu-RGD-BBN

To a solution of Boc-11-amino-3,6,9-trioxaundecanoic acid (Boc-NH-PEG₃-COOH, 40 mg, 0.13 mmol) and N,N-diisopropylethylamine (DIPEA, 20 μl) in ACN was added O—(N-succinimidyl)-1,1,3,3-tetramethyl-uronium tetrafluoroborate (TSTU, 27 mg, 0.09 mmol). The reaction mixture was stirred at room temperature for 0.5 h and then added to a solution of Glu-RGD-BBN (36 mg, 0.02 mmol) in N,N′-dimethylformamide (DMF). After being stirred at room temperature for 2 h, the Boc-protected PEG₃-Glu-RGD-BBN was isolated by preparative HPLC. The Boc group was then removed with anhydrous TFA and the crude product was again purified by preparative HPLC. The collected fractions were combined and lyophilized to afford 23 mg of PEG₃-Glu-RGD-BBN as a white fluffy powder (yield: 58%). (MALDI-TOF MS: m/z 1973.3 for [M+H]⁺ (C₈₉H₁₃₈N₂₅O₂₄S, Calcd. 1974.3)). RP-HPLC Rₜ=18.8 min.

### Synthesis of FB-PEG₃-Glu-RGD-BBN

N-Succinimidyl-4-fluorobenzoate (SFB, 4 mg, 16.8 μmol) and PEG₃-Glu-RGD-BBN (2 mg, 1.0 μmol) were mixed in 0.05 mol/L borate buffer (pH 8.5) at room temperature. After constant shaking for 2 h, the desired product FB-PEG₃-Glu-RGD-BBN was isolated by semi-preparative HPLC (1.6 mg, yield: 76%). Analytical HPLC (RP-HPLC Rₜ=23.3 min) and mass spectrometry (MALDITOF-MS: m/z 2095.9 for [M+H]⁺ (C₉₆H₁₄₁FN₂₅O₂₅S, calcd. 2095.4)) analyses confirmed the product identification.

### Radiochemistry

N-Succinimidyl-4-¹⁸F-fluorobenzoate (¹⁸F-SFB) was synthesized and purified with HPLC as we previously reported by modifying GE TRACERlab FX-FN module²⁷. The purified ¹⁸F-SFB was rotary evaporated to dryness, redissolved in dimethyl sulfoxide (DMSO, 200 μL), and added to a DMSO solution of PEG₃-Glu-RGD-BBN peptide (200 μg) and DIPEA (20 μL). The reaction mixture was incubated at 60° C. for 30 min. After dilution with 5% aqueous acetic acid solution (3 mL), the mixture was purified by semi-preparative HPLC. The desired fractions containing ¹⁸F-PEG₃-RGD-BBN were combined and rotary evaporated to dryness. The activity was then reconstituted in PBS and passed through a 0.22-μm Millipore filter into a sterile multidose vial for in vitro and in vivo experiments.

### Example 21

### Cell Binding Assay with ¹⁸F-PEG3Glu-RGD-BBN

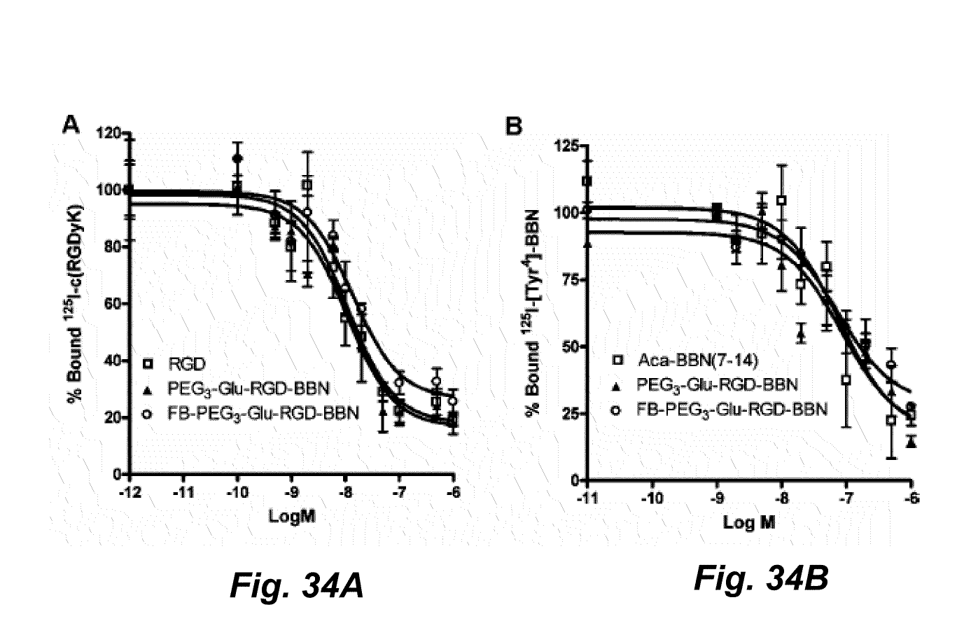

The integrin αᵥβ₃ receptor-binding affinities of cyclic RGD peptide c(RGDyK), PEG₃-Glu-RGD-BBN, and FB-PEG₃-Glu-RGD-BBN were determined by performing competitive binding assay with ¹²⁵I-c(RGDyK) as the radioligand. All peptides inhibited the binding of ¹²⁵I-c(RGDyK) to integrin expressing U87MG cells in a concentration-dependent manner. The IC₅₀ values for c(RGDyK), PEG₃-Glu-RGD-BBN, and FB-PEG₃-Glu-RGD-BBN were 11.19±1.44, 10.80±1.46, and 13.77±1.82 nM, respectively, as shown in FIGS. 34A and 34B.

The binding affinities of Aca-BBN(7-14), PEG₃-Glu-RGD-BBN, and FB-PEG₃-Glu-RGD-BBN for GRPR were evaluated using GRPR positive PC-3 cells with ¹²⁵I-[Tyr⁴]BBN as the radioligand. Results of the cell-binding assay were plotted in sigmoid curves for the displacement of ¹²⁵I-[Tyr⁴]BBN from PC-3 cells as a function of increasing concentration of BBN analogs. The IC₅₀ values were determined to be 78.96±2.12 nM for BBN, 85.45±1.95 nM for PEG₃-Glu-RGD-BBN, and 73.28±1.57 nM for FB-PEG₃-Glu-RGD-BBN on PC-3 cells (FIG. 34B). The comparable IC₅₀ values from these two sets of experiments suggest that the incorporation of PEG₃ spacer to Glu-RGD-BBN peptide possesses comparable GRPR and integrin αᵥβ₃ receptor-binding affinities as the corresponding unmodified monomers. Further coupling of 4-fluorobenzoyl group also had little effect on the integrin and GRPR receptor binding characteristics.

### Example 22

### Cell Uptake Studies with ¹⁸F-PEG₃-Glu-RGD-BBN

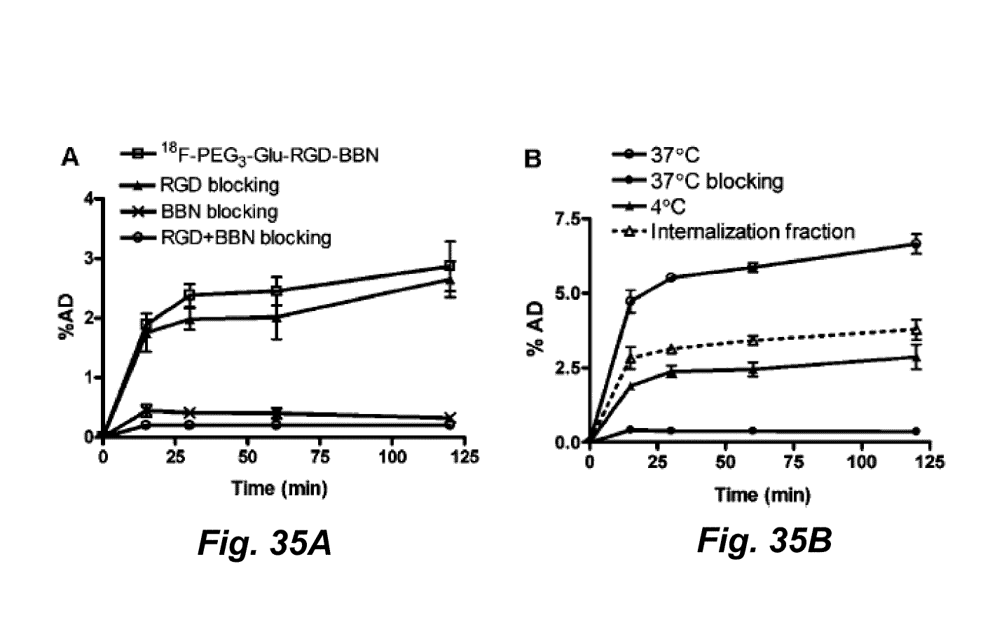

The cell uptake of ¹⁸F-FB-PEG₃-Glu-RGD-BBN was evaluated in PC-3 tumor cells that express high GRPR and moderate integrin levels. FIG. 35A shows the results at 4° C., in which the radiotracer only binds to the receptors on the cell surface without internalization. Rapid binding of ¹⁸F-FB-PEG₃-Glu-RGD-BBN to the cell surface was observed for the first 15 min of incubation. After 15 min, the radiotracer exhibited a small and steady increase with time. Cell surface binding of ¹⁸F-FB-PEG₃-Glu-RGD-BBN was partially inhibited in the presence of either RGD or Aca-BBN(7-14) peptide alone. The inhibition of BBN was more effective than RGD peptide. When both RGD and BBN were co-incubated with ¹⁸F-FB-PEG₃-Glu-RGD-BBN, the binding of ¹⁸F-FB-PEG₃-Glu-RGD-BBN with PC-3 cells was significantly inhibited to trace level. For example, at 60 min, the cell uptake of ¹⁸F-FB-PEG₃-Glu-RGD-BBN was inhibited by 17.55±8.89%, 83.67±2.09% and 91.97±0.14% by RGD, BBN and RGD+BBN, respectively (FIG. 35A).

The cell uptake of ¹⁸F-FB-PEG₃-Glu-RGD-BBN was significantly increased when incubated at 37° C. due to both cell-surface receptor binding and receptor mediated internalization. As shown in the FIG. 35B, the cell uptake of ¹⁸F-FB-PEG₃-Glu-RGD-BBN was 4.72±0.37%, 5.51±0.11%, 5.87±0.15% and 6.65±0.34% at 15 min, 30 min, 60 min and 120 min, respectively. The cell uptake was significantly inhibited by co-incubation with excess amount of Glu-RGD-BBN peptide heterodimer, indicating the specific uptake of the radiotracer in PC-3 tumor cells. The internalized fraction was calculated by subtracting the cell uptake at 4° C. from the uptake at 37° C. at each time point (dotted line in FIG. 35B). The internalized values were between the total uptake (37° C.) and the cell-surface binding (4° C.) at all time points. The internalization of ¹⁸F-FB-PEG₃-Glu-RGD-BBN was rapid, reaching about 3.5% within 30 min of incubation, and plateaus afterwards.

### Example 23

### MicroPET Imaging with ¹⁸F-PEG₃-Glu-RGD-BBN

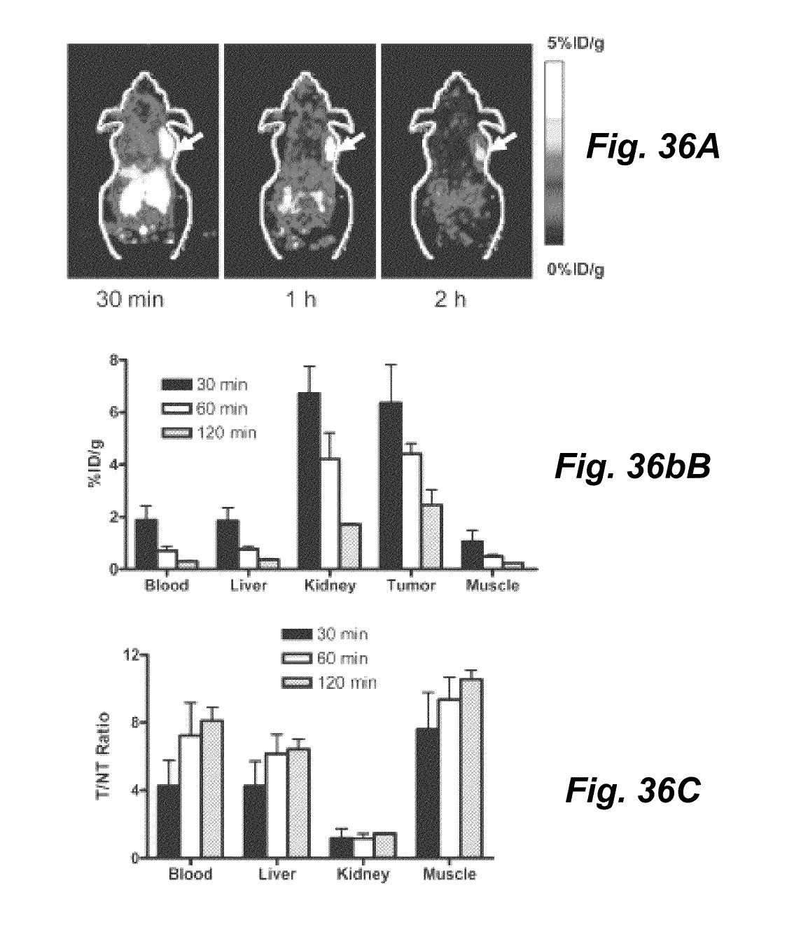

Representative coronal microPET images of PC-3 tumor-bearing mice (n=4) at different times after intravenous injection of 3.7 MBq (100 μCi) of ¹⁸F-FB-PEG₃-Glu-RGD-BBN are shown in FIG. 36A. The tumors were clearly visible with high contrast to contralateral background at all time points measured from 30 to 120 min. Prominent uptake was also observed in the kidneys at early time points, suggesting that this tracer is mainly excreted through the renal route.

Quantification of tumor and major organ activity accumulation in microPET scans was realized by measuring the regions of interest (ROIs) that encompassing the entire organ on the coronal images. The tumor uptake of ¹⁸F-FB-PEG₃-Glu-RGD-BBN was determined to be 6.35±2.52, 4.41±0.71, and 2.47±0.81% ID/g at 30, 60, and 120 min. The liver uptake was very low, the highest of which is less than 2% ID/g at 30 min post-injection (FIG. 36B). With the rapid clearance of the tracer from normal non-targeted organs, the tumor/non-tumor (T/NT) ratio increased with time. At 120 min post-injection, the T/NT ratios were 8.10±1.14 for blood, 6.43±0.81 for liver, 1.44±0.05 for kidneys and 10.54±0.75 for muscle, respectively (FIG. 36C).

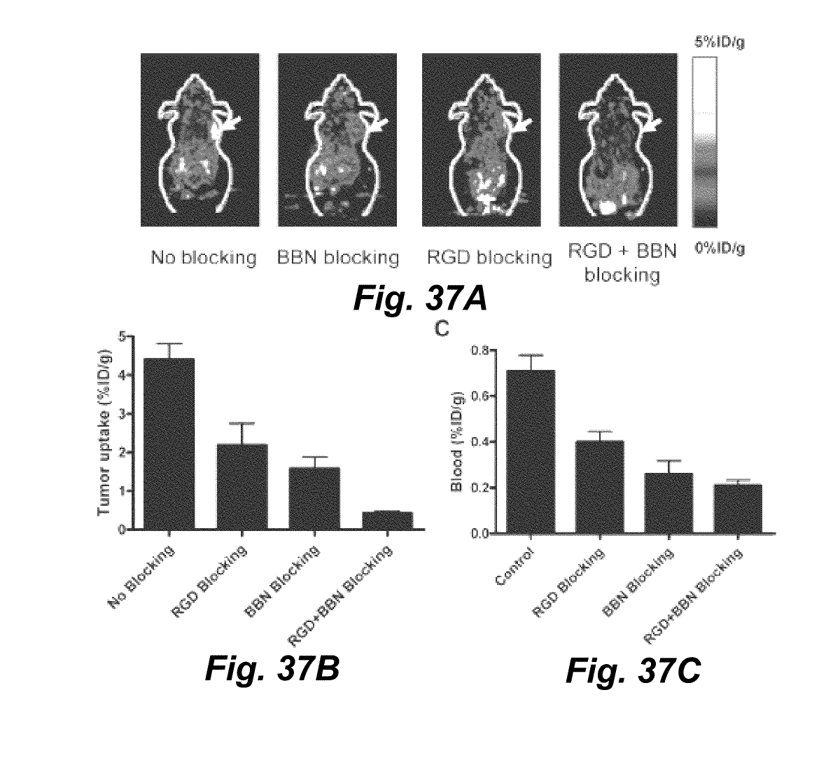

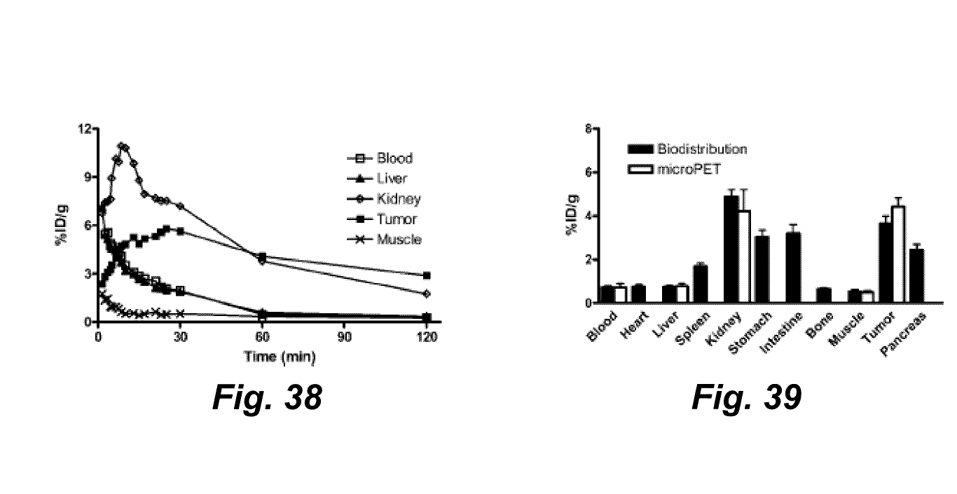

The integrin and GRPR dual-receptor binding specificity of ¹⁸F-FB-PEG₃-Glu-RGD-BBN in vivo was conformed by several blocking studies (FIG. 37A-37C). Representative coronal images of PC-3 tumor mice at 1 h post-injection of ¹⁸F-FB-PEG₃-Glu-RGD-BBN in the presence of RGD (10 mg/kg of c(RGDyK)), BBN (15 mg/kg of Aca-BBN(7-14)), or both RGD and BBN (10 mg/kg of RGD and 15 mg/kg of BBN) are illustrated in FIG. 37A. The tumor uptake ¹⁸F-FB-PEG₃-Glu-RGD-BBN (FIG. 38B) was partially inhibited by either RGD (2.19±0.97% ID/g, 50% decrease of tumor uptake, n=3) or BBN (1.58±0.52% ID/g, 65% decrease of the tumor uptake, n=3) alone. In contrast, when ¹⁸F-FB-PEG₃-Glu-RGD-BBN was co-administered with both RGD and BBN, the tumor uptake was significant inhibited to the background level (0.43±0.08% ID/g, 90% decrease of the tumor uptake, n=3).

The tumor targeting efficacy of ¹⁸F-FB-PEG₃-Glu-RGD-BBN in PC-3 tumor-bearing nude mice was also evaluated by 30 min dynamic microPET scanning followed by 5-min static scans at 1 h and 2 h postinjection. As shown in FIG. 38, the tracer cleared rapidly from the blood circulation (ROI at the heart). For example, the blood % ID/g at 30 min is only 35% of that at 2 min p.i. The PC-3 tumor uptake was 3.54, 4.86, 5.64, 4.08 and 2.88% ID/g at 5, 15, 30, 60 and 120 min p.i, respectively. The tracer was excreted mainly through the kidneys. The kidney uptake reached a peak at about 10 min p.i and then decreased with time. At 120 min p.i, the tumor uptake of the tracer was higher than any of the other normal organ.

To validate the accuracy of microPET quantification, a biodistribution study was performed in nude mice bearing PC-3 tumors. Each mouse was injected with 0.74 MBq (20 μCi) of ¹⁸F-FB-PEG₃-Glu-RGD-BBN and then sacrificed at 1 h p.i. (n=4). As shown in FIG. 39, the tumor uptake was 4.00±0.08% ID/g, and the kidney uptake was 4.87±0.67% ID/g. The uptake values in the blood, heart, liver, spleen, bone, and muscle were all less than 2% ID/g. ¹⁸F-FB-PEG₃-Glu-RGD-BBN showed relatively high uptake in the normal organs that express GRPR, such as stomach, small intestine and pancreas. Comparing the biodistribution and microPET quantification, there was no significant difference between the blood, liver, kidneys, tumor, and muscle (P>0.05; FIG. 39), suggesting that quantification of noninvasive microPET scans is a true reflection of the distribution of ¹⁸F-FB-PEG₃-Glu-RGD-BBN in these organs. Note that the pancreas was unable to be delineated from microPET due to the limit of the spatial resolution.

### Example 24

### Statistical Analysis

Quantitative data are expressed as mean±SD. Means were compared using 1-way ANOVA and the Student t test. P values of <0.05 were considered statistically significant.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

1. A composition comprising a heterodimeric probe, wherein the heterodimeric probe comprises:
a first peptide domain comprising a moiety having the characteristic of selectively binding to an integrin;
a second peptide domain comprising a moiety having the characteristic of selectively binding to a gastrin-releasing peptide receptor;
a linker connecting the first peptide domain and the second peptide domain;
and a prosthetic group.

2. The composition according to claim 1, wherein the first peptide domain comprises at least one tripeptide comprising the amino acid sequence arginine-glycine-aspartate (Arg-Gly-Asp).

3. The composition according to claim 1, wherein the moiety having the characteristic of selectively binding to an integrin comprising at least one peptide selected from the group consisting of: cyclo(Arg-Ala-Asp-D-Phe-Lys), cyclo(Arg-Ala-Asp-D-Phe-Val), cyclo(Arg-Ala-Asp-D-Tyr-Cys), cyclo(Arg-Ala-Asp-D-Tyr-Lys), cyclo(Arg-Gly-Asp-D-Phe-Cys), cyclo(Arg-Gly-Asp-D-Phe-Glu), cyclo(Arg-Gly-Asp-D-Phe-Lys), cyclo(Arg-Gly-Asp-D-Tyr-Cys), cyclo(Arg-Gly-Asp-D-Tyr-Glu), cyclo(Arg-Gly-Asp-D-Tyr-Lys), cyclo[Arg-Gly-Asp-D-Phe-Lys(Ac-SCH₂CO)], cyclo[Arg-Gly-Asp-D-Phe-Lys(H-Ser)], cyclo[Arg-Gly-Asp-D-Phe-Lys(PEG-PEG)], H-Glu[cyclo (Arg-Gly-Asp-D-Phe-Lys)]₂, H-Glu[cyclo(Arg-Gly-Asp-D-Phe-Lys)]₂, H-Glu[cyclo(Arg-Gly-Asp-D-Tyr-Lys)]₂, H-Gly-Arg-Ala-Asp-Ser-Pro-OH (SEQ ID NO.: 1), H-Gly-Arg-Gly-Asp-Asn-Pro-OH (SEQ ID NO.: 2), H-Gly-Arg-Gly-Glu-Ser-OH (SEQ ID NO.: 3), cyclo(Arg-Gly-Asp-D-Phe-Lys), H-Arg-Gly-Asp-Ser-Lys-OH (SEQ ID NO.: 4), H-Arg-Ala-Asp-Ser-Lys-OH (SEQ ID NO.: 5), Ac-Gly-D-Arg-Gly-Asp-Ser-Pro-Ala-Ser-Ser-Lys-(Gly)-4-Ser-D-Arg-(Leu)-6-D-Arg-NH₂, cyclo(Arg-Gly-Glu-D-Phe-Lys), and cyclo(Arg-Gly-Asp-D-Phe-Val).

4. The composition according to claim 1, wherein the moiety having the characteristic of selectively binding to an integrin comprises cyclo(arginine-glycine-aspartate-D-tyrosine-lysine).

5. The composition according to claim 1, wherein the first peptide domain comprises a multimer of conjugated peptides, wherein at least one peptide of the multimer of peptides comprises the amino acid sequence arginine-glycine-aspartate.

6. The composition according to claim 5, wherein the amino acid sequence of each peptide of the multimer of peptides comprises the amino acid sequence of arginine-glycine-aspartate.

7. The composition according to claim 5, wherein at least one peptide of the multimer of peptides comprises cyclo(arginine-glycine-aspartate-D-tyrosine-lysine).

8. The composition according to claim 1, wherein the moiety having the characteristic of selectively binding to a gastrin-releasing peptide receptor comprises a fragment of the polypeptide bombesin, wherein the fragment has an affinity for a gastrin-releasing peptide receptor.

9. The composition according to claim 8, wherein the moiety having the characteristic of selectively binding to a gastrin-releasing peptide receptor is selected from the group consisting of: bombesin(7-14) having the amino acid sequence of glutamine-tryptophan-alanine-valine-glycine-histidine-leucine-methionine (SEQ ID NO: 6), bombesin(8-14) having the amino acid sequence of asparagine-glutamine-tryptophan-alanine-valine-glycine-histidine-leucine-methionine (SEQ ID NO: 7), [Lys³]BBN (SEQ ID NO.: 8), [(D)Phe⁶, Leu-NHEt¹³, des-Met¹⁴]BN(6-14), (H-(D)Phe-Gln-Trp-Ala-Val-Gly-His-Leu-NH Et, and substituted variants of each, wherein the substituted variants of each have an affinity for a GRPR.

10. The composition according to claim 1, wherein the second domain is bombesin(7-14) and comprises the amino acid sequence of glutamine-tryptophan-alanine-valine-glycine-histidine-leucine-methionine (SEQ ID NO.: 6).

11. The composition according to claim 1, wherein the heterodimer probe selectively binds to the integrin αᵥβ₃.

12. The composition according to claim 1, wherein the heterodimer probe may selectively bind to the integrin αᵥβ₃ and gastrin-releasing peptide receptor.

13. The composition according to claim 1, wherein the linker connecting the first peptide domain and the second peptide domain comprises the formula (HOOC)—(CH₂)ₙ—(CHNH₂⁺)—(CH₂)ₘ—(COOH)ₐ, wherein n and m are each independently 0, or an integer from 1 to about 10, and a is an integer from 1 to about 10.

14. The composition according to claim 1, wherein the linker connecting the first peptide domain and the second peptide domain is selected from the group consisting of: (aspartate)ₓ, (glutamate)_{y}, wherein x and y are each independently integers from 1 to about 10, and a combination thereof.

15. The composition according to claim 1, wherein the linker connecting the first peptide domain and the second peptide domain is a glutamate residue or an aspartate residue.

16. The composition according to claim 1, wherein the linker further comprises a tether covalently bound thereto, and wherein the tether is between the linker and the prosthetic group.

17. The composition according to claim 1, wherein the linker comprises (Gly)ₙ, wherein n is an integer from 1 to about 12.

18. The composition according to claim 15, wherein the tether further comprises at least one polyethylene glycol moiety, and wherein the polyethylene glycol moiety has a molecular weight of about 200 to about 5000 daltons.

19. The composition according to claim 15, wherein the tether is a polyethylene glycol-3 (11-amino-3,6,9,-trioxaundecanoate) moiety.

20. The composition according to claim 1, wherein the prosthetic group comprises one or more of the following: a detectable label, a therapeutic agent, a reactive group capable of covalently bonding to a detectable label, a therapeutic agent, and a combination thereof.

21. The composition according to claim 1, wherein the prosthetic group comprises a detectable label, or a group capable of bonding to a detectable label.

22. The composition according to claim 21, wherein the group having the characteristic of bonding to a detectable label is selected from the group consisting of an amine group, a carboxyl group, and a metal chelating group.

23. The composition according to claim 22, wherein the metal chelating group is NOTA (1,4,7-triazacyclononane-1,4,7-triacetate) or DOTA (1,4,7,10-tetraazacyclododecane-N,N′,N″,N′″-tetracetate).

24. The composition according to claim 1, wherein the prosthetic group comprises a label from the group consisting of: a radiolabel, an optical label, and a radiolabel suitable for radiotherapy.

25. The composition according to claim 1, wherein the prosthetic group comprises a detectable label selected from the group consisting of: the fluoride isotope ¹⁸F, ⁶⁸Ga, ⁶⁴Cu, ⁸⁶Y, ¹²⁴I, ¹¹¹In, ^{99m}Tc, ^{123/131}I, a fluorescent dye, a quantum dot, an alpha emitter, a beta emitter, and a gamma emitter.

26. The composition according to claim 25, wherein the prosthetic group comprises a radionuclide selected from the group consisting of: ¹⁸F, ⁶⁸Ga, and ⁶⁴Cu.

27. The composition according to claim 26, wherein the prosthetic group is ¹⁸F-fluorobenzoate.

28. The composition according to claim 1, wherein the heterodimeric probe has a formula selected from the group consisting of:
formula I, formula II, formula III, formula IV, formula V, formula VI, formula VIII, formula VIII, formula VIIIa, and formula IX, wherein formula I, formula II, formula III, formula IV, formula V, formula VI, formula VIII, formula VIII, formula VIIIa, and formula IX have the structures as shown in FIGS. 7B, 1B, 10, 11, 7A, 9, 13, 22A, 22B, and 33 respectively, and wherein M+ is a metal ion.

29. The composition according to claim 28, wherein M+ is selected from the group consisting of: ⁶⁸Ga and ⁶⁴Cu.

30. The composition according to claim 1, further comprising a pharmaceutically acceptable carrier.

31. A method of identifying a cell or a population of cells expressing an integrin and a gastrin-releasing peptide receptor, comprising:
contacting a cell or population of cells with a composition, the composition comprising a heterodimeric probe having the characteristic of selectively binding to an integrin and to a gastrin-releasing peptide receptor of a cell;
allowing the heterodimeric polypeptide probe to selectively bind to at least one of an integrin and to a gastrin-releasing peptide receptor of a cell or a population of cells; and
detecting the presence of the heterodimeric probe on the cell or population of cells, whereby the presence of the heterodimeric probe on the cell or population of cells indicates that the cell or population of cells has an integrin, a gastrin-releasing peptide receptor, or both an integrin and a gastrin-releasing peptide receptor thereon.

32. The method of claim 31, wherein the cell or population of cells is a mammalian cell or population of mammalian cells, and wherein the cells or population of cells are isolated cells.

33. The method of claim 31, wherein the cell or population of cells is a mammalian cell or population of mammalian cells, and wherein the cells or population of cells are in a tissue of a human or animal host.

34. The method of claim 31, wherein the heterodimeric probe binds to the integrin αᵥβ₃ and gastrin-releasing peptide receptor.

35. The method of claim 31, wherein the composition comprising the heterodimeric probe is administered to an animal or human host.

36. The method of claim 31, wherein the heterodimeric Probe has a formula selected from the group consisting of:
formula I, formula II, formula IV, formula VII, formula VIIIa, and formula IX, wherein formula I, formula II, formula IV, formula VIII, formula VIIIa, and formula IX have the structures as shown in FIGS. 7B, 1B, 11, 13, 22B, and 33 respectively, and wherein M+ is a radionuclide selected from ⁶⁸Ga and ⁶⁴Cu.

37. The method of claim 31, wherein the heterodimeric probe is detected by positron emission tomography or by single photon emission computed tomography.

38. The method of claim 31, wherein the heterodimeric probe is admixed with a pharmaceutically acceptable carrier.

39. A method of imaging a tissue in an animal or human host comprising the steps of:
administering to an animal or human host a heterodimeric probe, wherein the probe has a detectable label thereon;
detecting the presence of the detectable label in the animal or human host; and
identifying a tissue in the animal or human host wherein the amount of the detectable label in the tissue is greater than in other tissues of the host, thereby determining the position of a tissue binding to the heterodimeric probe within the animal or human host.

40. The method according to claim 39, wherein the heterodimeric probe is selected from the group consisting of:
formula I, formula II, formula IV, formula VII, formula VIIIa, and formula IX, wherein formula I, formula II, formula IV, formula VII, formula VIIIa, and formula IX have the structures as shown in FIGS. 7B, 1B, 11, 13, 22B, and 33 respectively, and wherein M+ is a radionuclide selected from ⁶⁸Ga and ⁶⁴Cu.

41. The method of claim 40, wherein the heterodimeric probe is detected by positron emission tomography or by single photon emission computed tomography.

42. The method according to claim 40, wherein the heterodimeric probe selectively binds to a tumor in the animal or human host, wherein the tumor comprises cells expressing αᵥβ₃ and/or GRPR.

43. The method according to claim 42, wherein the tumor is a tumor of the breast, the prostate, a malignant melanoma, an ovarian carcinoma, a gastrointestinal carcinoma, or a glioblastoma.

44. A method of delivering an agent to a cell, comprising
contacting a cell or population of mammalian cells with a heterodimeric probe having the characteristic of simultaneously binding to two an integrin and to a gastrin-releasing peptide receptor, and wherein the probe further comprises an agent to be delivered to a target cell or tissue of a mammalian subject; and
allowing the heterodimeric probe to bind to an integrin, a gastrin-releasing peptide receptor, or both an integrin and a gastrin-releasing peptide receptor, on the cell or population of mammalian cells, thereby delivering the agent to the cell or cells.

45. The method according to claim 44, wherein the cell or population of cells is a mammalian cell or population of mammalian cells, and wherein the cells or population of cells are isolated cells.

46. The method according to claim 44, wherein the cell or population of cells is a mammalian cell or population of mammalian cells, and wherein the cells or population of cells are in a tissue of a human or animal host.

47. The method according to claim 44, wherein the agent is a therapeutic agent or a detectable agent.

48. The method according to claim 31, wherein the heterodimeric probe comprises:
a first peptide domain comprising a moiety having the characteristic of selectively binding to an integrin, and wherein the first peptide domain comprises at least one tripeptide comprising the amino acid sequence arginine-glycine-aspartate (Arg-Gly-Asp);
a second peptide domain comprising a moiety having the characteristic of selectively binding to a gastrin-releasing peptide receptor, wherein the moiety having the characteristic of selectively binding to a gastrin-releasing peptide receptor comprises a fragment of the polypeptide bombesin;
a linker connecting the first peptide domain and the second peptide domain;
a prosthetic group;
and optionally a tether covalently bound thereto, and wherein the tether is between the linker and the prosthetic group.

49. The method according to claim 39, wherein the heterodimeric probe comprises:
a first peptide domain comprising a moiety having the characteristic of selectively binding to an integrin, and wherein the first peptide domain comprises at least one tripeptide comprising the amino acid sequence arginine-glycine-aspartate (Arg-Gly-Asp);
a second peptide domain comprising a moiety having the characteristic of selectively binding to a gastrin-releasing peptide receptor, wherein the moiety having the characteristic of selectively binding to a gastrin-releasing peptide receptor comprises a fragment of the polypeptide bombesin;
a linker connecting the first peptide domain and the second peptide domain;
a prosthetic group;
and optionally a tether covalently bound thereto, and wherein the tether is between the linker and the prosthetic group.

50. The method according to claim 44, wherein the heterodimeric probe comprises:
a first peptide domain comprising a moiety having the characteristic of selectively binding to an integrin, and wherein the first peptide domain comprises at least one tripeptide comprising the amino acid sequence arginine-glycine-aspartate (Arg-Gly-Asp);
a second peptide domain comprising a moiety having the characteristic of selectively binding to a gastrin-releasing peptide receptor, wherein the moiety having the characteristic of selectively binding to a gastrin-releasing peptide receptor comprises a fragment of the polypeptide bombesin;
a linker connecting the first peptide domain and the second peptide domain;
a prosthetic group;
and optionally a tether covalently bound thereto, and wherein the tether is between the linker and the prosthetic group.


SEQUENCE LISTING (8 sequences)

SEQ ID NO: 1 - protein, 6 aa, Artificial sequence
  Synthetic construct
GRADSP

SEQ ID NO: 2 - protein, 6 aa, Artificial sequence
  Synthetic construct
GRGDNP

SEQ ID NO: 3 - protein, 5 aa, Artificial sequence
  Synthetic construct
GRGES

SEQ ID NO: 4 - protein, 5 aa, Artificial sequence
  Synthetic construct
RGDSK

SEQ ID NO: 5 - protein, 5 aa, Artificial sequence
  Synthetic construct
RADSK

SEQ ID NO: 6 - protein, 8 aa, Artificial sequence
  Synthetic construct Bombesin(BBN)(7-14)
QWAVGHLM

SEQ ID NO: 7 - protein, 9 aa, Artificial sequence
  Synthetic construct Bombesin(BBN)(8-14)
NQWAVGHLM

SEQ ID NO: 8 - protein, 11 aa, Artificial sequence
  Synthetic construct K3-BBN(7-14)
KKKQWAVGHLM